SEQUENCE LISTING (197 sequences)

SEQ ID NO: 1 - DNA, 3242 bp, Prunus dulcis
  source 1..3242: mol_type = genomic DNA
atggcgacgttgagacagtcattcgctttccttgttctagcttttgctttcttcttgtgtttcattatgagcactggtgggttgcattacaattcttttgctttttaaattctgtatgcatacaattagcatcgcattttatacttctattttatgtttagagaaatatatatggatttatcaggtaagggaggacttgatctagcgcacaactttctttggatgaatgactatttaggaattacatttctgcatggttaatttatttggtctactctgatagttgttgcaataagctcccctccataagtgcagtgttcatcatttcaagctaaacctatgtactttatacatcaaatccttaattaagataccattaaccttctcacaataattttcgcaggatcttacgtgtattttcaatttgtgcaacaatggccaccgacgacctgcagacttagcagcaaaccttccaaccaacaccggccattacaaagattcaccatccatggcctatggccaagtaactattcaaacccaaggaagcccagtaattgcaatgggtcacaatttaactttatgaaagtggtatgtattgtttcttttctttttcacttacttttagtatttagtttttagaaacttttagattgtatctaaagacaataatatacaatttttttaatgatttttttttaatcacaaatggtccttcaggttttcgaaattatcaccttttattctaaaagttttttttgtgacactaatgatttttaaggttactctctacacatcaaaatggtcattgccattagcttccgtcaaattttatgttaaattgataatgtggcacatatgtggagcctacacatacaatagtatatagtcacatgactttaactataatatataatatattttaaaacttaaaatccatataaaaattttaaaaagaaaattaagtctttttataaatataaaattattaaaactaaaaaactaaaaatcctgcagccatccctcatcgcagacccactcatcgagcaccttaagccaaggaattccaagagctacaagtgttcgagaaggaaatggcctctcgatttgcggattttgaagccaaagagttgaactttaacttggtgatgggaactaaagccaaggaattgcagggcattgtacaggaggttgagccaaacaaacaaatattgattgaagaatatgacagagagatgaaatccaacgagcagaaacttggttcgattcagaaattagtgttggagtactcgaatgcatttgaatcgaaaataaaatattttaatttgtttgagagtgagtctgcgatttggggaggggcgctgggagagggggaacgtagaggatggttgcaggtttttttagttttttatttataaaaagacttaattttcttttaaattttttatatggattttaagttttaaaatatttaaaatatattttatattttatttaaagtcacatggttctataccgttggatgtgtgggcttcacatatgtgccacatcatcaatttaacataaaatttgacgaaagctaaggcagggacaattttgatgtgtggagggtaacgttaaatattattagtgtcacaaaaaaactttagggataaaaggtgataatttcacaaacttgaaagaccatttgtgattaaaaaaaaaaaagaagcctttttttaaatttatacaagtgataacatactcaaaattaataaaccttgggtattggataaattttggtgttggtcctagatgcattatttttaactagactgacctttttttttcctccttgtattgcgatagagatttttctggggtaaatttacatttatgtataactagcccttggcacacggtgtgccaattagttttttattttattttattgtattttgtttaagaattaggagagagaatcccaaaaaaaaaaacaagttcttattttaaattttttttatttactacattatacttatttaatgaaattttttaatacttaataaattcattaataaatgataatttttggtattttgaatattttatgattctctgtcttttgctttagatataaagataaaataaaaaagacacgaaaagaaatagtttttttttgttgaagtagaattttcattctacataagcaatacaagataaaagaaaggtcgttatgctagttagtcggctttaattagttataattttatttaattactggtttttaatattttgtccttaaaatattaaattgtggttgtttgataaactagttaaccaagtgtataaaaaaaaagtcaactagcaaaggaccaactagtgtagtggtttggagtatttattccctcaggcaagatcctggattcaagtcctagcatccgtgttgtgcgtgtgagtttaatatgctatcgtccctttcaataggaaaggtcctccaaaaaaaaaaaaaaaaaaaaggtcaaccaatgattcttttaagatattaaattttgtgaatgtactattttattcctatttaatgtgcttacctatatataatgaatctgctcttctaattaaacgacctacaattttatactgtttgtgtatatattcaaagtattgtactttatatatatatatatatatatatatatatatatatcaacaaggtattgtacttaaatgaattaaaatcaaaattcaacttaaaacccagataaatgcaaaaaatatatatatttgtcacttgagattttatttttctcgaaatttatgtatgtattgcttgcatgtctcagtatcctcagttgcgaaccaaactgaagagatcttggccggacgtggaaggtggcaatgatacaaagttttgggaaggcgaatggaacaagcatggcacatgttccgaacggacgcttaaccaaatgcaatacttcgaggtatcccacgccatgtggaggtcgtacaatattacaaatatccttaaagacgctcatatcgtaccaaatccaacacaaagatggaagtactcggacatagtatcacccattaaaacagcaactggaagaacacccacccttcgttgcaaaactgatccagcaatgcctaacaactctcagttgttacatgaagtggtattttgttatggatataatgcaaaactgcatattgactgtaatcgaacagcaggatgccgaaatcatattgacatcttgtttcaataa

SEQ ID NO: 2 - DNA, 684 bp, Prunus dulcis
  source 1..684: mol_type = genomic DNA
atggcgacgttgagacagtcattcgctttccttgttctagcttttgctttcttcttgtgtttcattatgagcactggatcttacgtgtattttcaatttgtgcaacaatggccaccgacgacctgcagacttagcagcaaaccttccaaccaacaccggccattacaaagattcaccatccatggcctatggccaagtaactattcaaacccaaggaagcccagtaattgcaatgggtcacaatttaactttatgaaagtgtatcctcagttgcgaaccaaactgaagagatcttggccggacgtggaaggtggcaatgatacaaagttttgggaaggcgaatggaacaagcatggcacatgttccgaacggacgcttaaccaaatgcaatacttcgaggtatcccacgccatgtggaggtcgtacaatattacaaatatccttaaagacgctcatatcgtaccaaatccaacacaaagatggaagtactcggacatagtatcacccattaaaacagcaactggaagaacacccacccttcgttgcaaaactgatccagcaatgcctaacaactctcagttgttacatgaagtggtattttgttatggatataatgcaaaactgcatattgactgtaatcgaacagcaggatgccgaaatcatattgacatcttgtttcaataa

SEQ ID NO: 3 - protein, 227 aa, Prunus dulcis
MATLRQSFAFLVLAFAFFLCFIMSTGSYVYFQFVQQWPPTTCRLSSKPSNQHRPLQRFTIHGLWPSNYSNPRKPSNCNGSQFNFMKVYPQLRTKLKRSWPDVEGGNDTKFWEGEWNKHGTCSERTLNQMQYFEVSHAMWRSYNITNILKDAHIVPNPTQRWKYSDIVSPIKTATGRTPTLRCKTDPAMPNNSQLLHEVVFCYGYNAKLHIDCNRTAGCRNHIDILFQ

SEQ ID NO: 4 - DNA, 1131 bp, Prunus dulcis
  source 1..1131: mol_type = genomic DNA
atgacattcgcactacgtaagaaagaaatcttaatcgacatcctagtaagactacctgcaaaatcccttgttcgatttctgtgtacatgcaagtcatggagtgatttgattggcagctcgagttttgttggcacacaccttcataggaatgtcacgggacatgcccaggcctatctcctttgcctccaccacccaaattttgaatgtcaaagggatgatgatgacccatattttaaagaagaactccaatggtcactgttttccaatgttacatttgaagagagctccaagttaagccatcccttagggagcacagaacattatgtgatatatggttcaagcaatggtttagtttgcatatcggatgagataatgaatttcgatagtcctatacacatatggaacccatcggttaagaaacttagaaccactccaatcagcaccagcattaacattaaatttagccatgttgctctccaattcggtttccactccggggttaatgactacagggctgtaaggatgttgcgtacaaaccaaaatgccttggcggttgagatttatagtctcagaacagactcttggacgatgattgaagcaattcctccttggttaaaatgcacttggcagcatcatcagggtacattttttaatggagtagcataccacatcattgagaaaggtcctacattcagtgttatatcattcgattcaggcagtgaagaatttgaagaattcatagcaccagatgccatttgcagtttatggaggttatgcatccacgtttacaaggagcaaatttgcttgctttttggatattatagttgtgaggaggagggcatggaaaacattgacttatgggttctgcaagaaaaacggtggaaacaattgtgtccttttatttatgatcccttggatgatttctatcagataatcgggattagtactgacaataaactcataatggcaagaaaagatttcagtaagggcggagtagaactgcaattggggaattacgaatccaaacaagttcttgaaacagggattaagttggccatcatgagatatggcgaaagcgaattcttgtttgcaatgacttacgtagaaagtttagctttactcaataactag

SEQ ID NO: 5 - protein, 376 aa, Prunus dulcis
MTFALRKKEILIDILVRLPAKSLVRFLCTCKSWSDLIGSSSFVGTHLHRNVTGHAQAYLLCLHHPNFECQRDDDDPYFKEELQWSLFSNVTFEESSKLSHPLGSTEHYVIYGSSNGLVCISDEIMNFDSPIHIWNPSVKKLRTTPISTSINIKFSHVALQFGFHSGVNDYRAVRMLRTNQNALAVEIYSLRTDSWTMIEAIPPWLKCTWQHHQGTFFNGVAYHIIEKGPTFSVISFDSGSEEFEEFIAPDAICSLWRLCIHVYKEQICLLFGYYSCEEEGMENIDLWVLQEKRWKQLCPFIYDPLDDFYQIIGISTDNKLIMARKDFSKGGVELQLGNYESKQVLETGIKLAIMRYGESEFLFAMTYVESLALLNN

SEQ ID NO: 6 - DNA, 2472 bp, Prunus dulcis
  source 1..2472: mol_type = genomic DNA
atggggatgttgaaatcgtcactcgctttccttgttcttggttttgctttcttcttttgttacgttatgagcagtggtgggttaaattacaatcttttgctatatgcatataatcagctttgcatttttctacttgtatttgttgttcagagaaactattgtgtgtattggatgatatatcacatgacatgcggtgtattgaattaacccacatatttttcatttaatctaacgcacaactttctttggatgagtaagtatttggggattgtttttgttttcattatcttttgtttattctgataattggttgcaataagtgcagtctattcatcacaataattttggcaggatcttatgactattttcaatttgtgcaacaatggccaccgaccaactgcagagttcgcatgaagcgaccttgctccaatccccggccattacaatatttcacaatccatggcctatggccaagtaatttttcaaacccaacgaagcccagtaattgcaatgggactaaatttgatgcaaggaaagtggtacgtattgattttcttttcccaccttagcatttagtttttagaaaattgattgtcatatgaagatatcatgcttttcagtaaacagcagccatgacactggtgaaaattagaaagttcttcccctcgatatgttttcacgtttgcttctcctagtgcttccctactttcggccctctctctaccgtcccctctccctgggctctccacaggcactccatgaacacactccattctccatatatcttcctccgttaaataaacaccatcgttcctattgtctaaactttaatagagaacaagaaaaggatggaaaaacaaatctggcgatgaagtataatgtcacgccaaaaagagatgcgaagaacatgaatgcatttcaattaaacaaataatttcttattaacttattgttactaaacccttggcacacactgtgccaattggtttttttattggaaaatactttccccttaccaggtaagtatgagctcttacattcctgccaatagtagtatgatgtgtgcaaaagtttttcttttatgtttttgatgaattatttttgcatatgtgtcaaaatgtgattcgcaagaaggaagagtatctccttactagtaaggaggcaagtagtatcattttttatttaagaattaagaaagagaatataacaacaacaacaacaatataacatcgtgttgcatgaaaataggacccatgttcttattttggtttttatttgaacttttttaaatatgaaaaaatattaatttgctatattatccctatttaatgaaaagtttcaatagttaataaatcaataactaagggaaatttttagtattttgaatgttttattctctactttttgcttatgtatgtagataaattcgttaattaagggcaatttttggtattttgaatgttttattctctgccttttgcttgtacgcttgagcatatgtgccaattggttttttaaattttattttagaattaaaaaagataatgggtaagtgttccacaaaaataggacctattatttgattttgttttaaattttaaattttgtaatatgaaaaagtgtgaatgtaccatattatcctcatttaattaataatttcaattcttaatgtttatattaaccaatgtcattttctgatattttgaatgtttcaccgttctctactttttgcttcatatatataaatatagataaattcagtaactaagggcaaattttggtattttgaatattttaccactctctgtattttgcttcatatatatagatatagataaattcattaattacggtcaaattttggtattttgaatgttttaccaatctttgccttttgaatatatatagatgaactaaaatccaagaaagaaaacttaagtatatatctaaatagtgtacatcaaaattaaattctgtataatactcaggtttaatgaagaaaaaaaatcttacccgagtctttactttttctcatgatatgtatatgttgattcgatgtctcagtatcctgaaatgcgatccgatctgaagatatcttggccagacgtggaaagtggcaatgatacaaaattttgggaagacgaatggaacaaacacggtacatgttctgaacagacacttaatcaattccagtactttgagcgatcccacgaaatgtggatgtcgtacaatattacagagatccttaaaaacgcttcaatcgtaccacatccggcaaaaacatggacctattcggacatagtatcacccatcaaagcagcaactggaagaacaccgcttcttcgttgcaaatacgataacaacactcagttgttacatgaagtggtattttgttatggctataaagcaataaagcagattgactgtaatcggccaggatgcaaaaataaaattgacatcaagtttcaataa

SEQ ID NO: 7 - DNA, 672 bp, Prunus dulcis
  source 1..672: mol_type = genomic DNA
atggggatgttgaaatcgtcactcgctttccttgttcttggttttgctttcttcttttgttacgttatgagcagtggatcttatgactattttcaatttgtgcaacaatggccaccgaccaactgcagagttcgcatgaagcgaccttgctccaatccccggccattacaatatttcacaatccatggcctatggccaagtaatttttcaaacccaacgaagcccagtaattgcaatgggactaaatttgatgcaaggaaagtgtatcctgaaatgcgatccgatctgaagatatcttggccagacgtggaaagtggcaatgatacaaaattttgggaagacgaatggaacaaacacggtacatgttctgaacagacacttaatcaattccagtactttgagcgatcccacgaaatgtggatgtcgtacaatattacagagatccttaaaaacgcttcaatcgtaccacatccggcaaaaacatggacctattcggacatagtatcacccatcaaagcagcaactggaagaacaccgcttcttcgttgcaaatacgataacaacactcagttgttacatgaagtggtattttgttatggctataaagcaataaagcagattgactgtaatcggccaggatgcaaaaataaaattgacatcaagtttcaataa

SEQ ID NO: 8 - protein, 223 aa, Prunus dulcis
MGMLKSSLAFLVLGFAFFFCYVMSSGSYDYFQFVQQWPPTNCRVRMKRPCSNPRPLQYFTIHGLWPSNFSNPTKPSNCNGTKFDARKVYPEMRSDLKISWPDVESGNDTKFWEDEWNKHGTCSEQTLNQFQYFERSHEMWMSYNITEILKNASIVPHPAKTWTYSDIVSPIKAATGRTPLLRCKYDNNTQLLHEVVFCYGYKAIKQIDCNRPGCKNKIDIKFQ

SEQ ID NO: 9 - DNA, 1125 bp, Prunus dulcis
  source 1..1125: mol_type = genomic DNA
atgacattcaaattaggtaagaaagaaatcttgatcgacatcctagcaagactaactgcaaaatccctggttcggtttctttgtacatgcaagtcatggagtgatttgattggcaggtccagttttgtcagcacacacattaataggaatgtcactaaacatgcccacgtctatctactttgcctccaccacccaacttttgaacgtcaaaatgacaacgatgacccatttgttgaagaagaacttctatggtcacttttttcaaatgaaacatttgagcagttctctaagttaagcaatcccttaggaagcacagaacattatgggatatatggttcaagcaatggtctagtttgcatttcggacgagatattgaattatgatagtcctatacacatatggaacccatcggttaggaaatttaggacccctccaataagccccaacaacattaaatttgtctacgttgctctccaattcggcttccactcgggggttaatgactacaaggctgtaagaatgatgcgttccaacaaagataccttcgcggttgaggtttatagtcttagaacagactcttggaagatgattgaaacaattcctccttggttaaaatgcacttggcagcatcatacgggtacattttttaatggagcggcataccacatcattgagaaaggtcccttattcagcattatgtccttcgattcaggcagtgaagagttcgaagaattcattgcaccagatgccatttgcaattcacgggggttgtgtatcgacgcttacaaggaccaaatttgcttgcttttcagattttatggttgtgaggaggagggcatgcgaaaagttgacttgtgggttctgcaagaaaaacgatggaaacaattgtgtccttttattattccttggaaatattgccgtacaattgggactagtatagataatgagctcttattgcaaaaaaaagatttcaataagggcgcagcagatctatgtttgtgtaattacgaatccaagcaagttcttgaaacaggaattaagttggccatcatgacatatggcgaaatcgatttcttgttttcatttacttacatagaaagtttggttttactcaataattattaa

SEQ ID NO: 10 - protein, 374 aa, Prunus dulcis
MTFKLGKKEILIDILARLTAKSLVRFLCTCKSWSDLIGRSSFVSTHINRNVTKHAHVYLLCLHHPTFERQNDNDDPFVEEELLWSLFSNETFEQFSKLSNPLGSTEHYGIYGSSNGLVCISDEILNYDSPIHIWNPSVRKFRTPPISPNNIKFVYVALQFGFHSGVNDYKAVRMMRSNKDTFAVEVYSLRTDSWKMIETIPPWLKCTWQHHTGTFFNGAAYHIIEKGPLFSIMSFDSGSEEFEEFIAPDAICNSRGLCIDAYKDQICLLFRFYGCEEEGMRKVDLWVLQEKRWKQLCPFIIPWKYCRTIGTSIDNELLLQKKDFNKGAADLCLCNYESKQVLETGIKLAIMTYGEIDFLFSFTYIESLVLLNNY

SEQ ID NO: 11 - RNA, 20 nt, Prunus dulcis
  source 1..20: mol_type = genomic RNA
accatccatggcctatggcc

SEQ ID NO: 12 - RNA, 20 nt, Prunus dulcis
  source 1..20: mol_type = genomic RNA
gccataggccatggatggtg

SEQ ID NO: 13 - RNA, 20 nt, Prunus dulcis
  source 1..20: mol_type = genomic RNA
aatttgtgcaacaatggcca

SEQ ID NO: 14 - RNA, 20 nt, Prunus dulcis
  source 1..20: mol_type = genomic RNA
cgcaggatcttacgtgtatt

SEQ ID NO: 15 - RNA, 20 nt, Prunus dulcis
  source 1..20: mol_type = genomic RNA
ctgctaagtctgcaggtcgt

SEQ ID NO: 16 - RNA, 20 nt, Prunus dulcis
  source 1..20: mol_type = genomic RNA
taatggccggtgttggttgg

SEQ ID NO: 17 - RNA, 20 nt, Prunus dulcis
  source 1..20: mol_type = genomic RNA
gtattttctaagtatggcga

SEQ ID NO: 18 - RNA, 20 nt, Prunus dulcis
  source 1..20: mol_type = genomic RNA
taagtatggcgacgttgaga

SEQ ID NO: 19 - RNA, 20 nt, Prunus dulcis
  source 1..20: mol_type = genomic RNA
ctttcttcttgtgtttcatt

SEQ ID NO: 20 - RNA, 20 nt, Prunus dulcis
  source 1..20: mol_type = genomic RNA
ttcttgtgtttcattatgag

SEQ ID NO: 21 - RNA, 20 nt, Prunus dulcis
  source 1..20: mol_type = genomic RNA
ataaagttaaattgtgaccc

SEQ ID NO: 22 - RNA, 20 nt, Prunus dulcis
  source 1..20: mol_type = genomic RNA
actttatgaaagtggtatgt

SEQ ID NO: 23 - RNA, 20 nt, Prunus dulcis
  source 1..20: mol_type = genomic RNA
taatattgtacgacctccac

SEQ ID NO: 24 - RNA, 20 nt, Prunus dulcis
  source 1..20: mol_type = genomic RNA
aggatatttgtaatattgta

SEQ ID NO: 25 - RNA, 20 nt, Prunus dulcis
  source 1..20: mol_type = genomic RNA
atgggtgatactatgtccga

SEQ ID NO: 26 - RNA, 20 nt, Prunus dulcis
  source 1..20: mol_type = genomic RNA
ttgaaacaagatgtcaatat

SEQ ID NO: 27 - RNA, 20 nt, Prunus dulcis
  source 1..20: mol_type = genomic RNA
gttcgcaactgaggatactg

SEQ ID NO: 28 - RNA, 20 nt, Prunus dulcis
  source 1..20: mol_type = genomic RNA
gttaagcgtccgttcggaac

SEQ ID NO: 29 - RNA, 20 nt, Prunus dulcis
  source 1..20: mol_type = genomic RNA
cattatatccataacaaaat

SEQ ID NO: 30 - RNA, 20 nt, Prunus dulcis
  source 1..20: mol_type = genomic RNA
ggcatcctgctgttcgatta

SEQ ID NO: 31 - DNA, 20 bp, Prunus dulcis
  source 1..20: mol_type = genomic DNA
accatccatggcctatggcc

SEQ ID NO: 32 - DNA, 20 bp, Prunus dulcis
  source 1..20: mol_type = genomic DNA
gccataggccatggatggtg

SEQ ID NO: 33 - DNA, 20 bp, Prunus dulcis
  source 1..20: mol_type = genomic DNA
aatttgtgcaacaatggcca

SEQ ID NO: 34 - DNA, 20 bp, Prunus dulcis
  source 1..20: mol_type = genomic DNA
cgcaggatcttacgtgtatt

SEQ ID NO: 35 - DNA, 20 bp, Prunus dulcis
  source 1..20: mol_type = genomic DNA
ctgctaagtctgcaggtcgt

SEQ ID NO: 36 - DNA, 20 bp, Prunus dulcis
  source 1..20: mol_type = genomic DNA
taatggccggtgttggttgg

SEQ ID NO: 37 - DNA, 20 bp, Prunus dulcis
  source 1..20: mol_type = genomic DNA
gtattttctaagtatggcga

SEQ ID NO: 38 - DNA, 20 bp, Prunus dulcis
  source 1..20: mol_type = genomic DNA
taagtatggcgacgttgaga

SEQ ID NO: 39 - DNA, 20 bp, Prunus dulcis
  source 1..20: mol_type = genomic DNA
ctttcttcttgtgtttcatt

SEQ ID NO: 40 - DNA, 20 bp, Prunus dulcis
  source 1..20: mol_type = genomic DNA
ttcttgtgtttcattatgag

SEQ ID NO: 41 - DNA, 20 bp, Prunus dulcis
  source 1..20: mol_type = genomic DNA
ataaagttaaattgtgaccc

SEQ ID NO: 42 - DNA, 20 bp, Prunus dulcis
  source 1..20: mol_type = genomic DNA
actttatgaaagtggtatgt

SEQ ID NO: 43 - DNA, 20 bp, Prunus dulcis
  source 1..20: mol_type = genomic DNA
taatattgtacgacctccac

SEQ ID NO: 44 - DNA, 20 bp, Prunus dulcis
  source 1..20: mol_type = genomic DNA
aggatatttgtaatattgta

SEQ ID NO: 45 - DNA, 20 bp, Prunus dulcis
  source 1..20: mol_type = genomic DNA
atgggtgatactatgtccga

SEQ ID NO: 46 - DNA, 20 bp, Prunus dulcis
  source 1..20: mol_type = genomic DNA
ttgaaacaagatgtcaatat

SEQ ID NO: 47 - DNA, 20 bp, Prunus dulcis
  source 1..20: mol_type = genomic DNA
gttcgcaactgaggatactg

SEQ ID NO: 48 - DNA, 20 bp, Prunus dulcis
  source 1..20: mol_type = genomic DNA
gttaagcgtccgttcggaac

SEQ ID NO: 49 - DNA, 20 bp, Prunus dulcis
  source 1..20: mol_type = genomic DNA
cattatatccataacaaaat

SEQ ID NO: 50 - DNA, 20 bp, Prunus dulcis
  source 1..20: mol_type = genomic DNA
ggcatcctgctgttcgatta

SEQ ID NO: 51 - RNA, 19 nt, Prunus dulcis
  source 1..19: mol_type = genomic RNA
aaccaaggaggaattgctt

SEQ ID NO: 52 - RNA, 20 nt, Prunus dulcis
  source 1..20: mol_type = genomic RNA
caccgtttttcttgcagaac

SEQ ID NO: 53 - RNA, 20 nt, Prunus dulcis
  source 1..20: mol_type = genomic RNA
ttaaccgatgggttccatat

SEQ ID NO: 54 - RNA, 20 nt, Prunus dulcis
  source 1..20: mol_type = genomic RNA
aatgtcaaagggatgatgat

SEQ ID NO: 55 - RNA, 20 nt, Prunus dulcis
  source 1..20: mol_type = genomic RNA
caatgttacatttgaagaga

SEQ ID NO: 56 - RNA, 20 nt, Prunus dulcis
  source 1..20: mol_type = genomic RNA
aagagagctccaagttaagc

SEQ ID NO: 57 - RNA, 20 nt, Prunus dulcis
  source 1..20: mol_type = genomic RNA
cactccggggttaatgacta

SEQ ID NO: 58 - RNA, 20 nt, Prunus dulcis
  source 1..20: mol_type = genomic RNA
tggaggttatgcatccacgt

SEQ ID NO: 59 - RNA, 20 nt, Prunus dulcis
  source 1..20: mol_type = genomic RNA
tggatattatagttgtgagg

SEQ ID NO: 60 - RNA, 20 nt, Prunus dulcis
  source 1..20: mol_type = genomic RNA
ctccttgtaaacgtggatgc

SEQ ID NO: 61 - RNA, 20 nt, Prunus dulcis
  source 1..20: mol_type = genomic RNA
ggtttgtacgcaacatcctt

SEQ ID NO: 62 - RNA, 20 nt, Prunus dulcis
  source 1..20: mol_type = genomic RNA
gtttgtacgcaacatcctta

SEQ ID NO: 63 - RNA, 20 nt, Prunus dulcis
  source 1..20: mol_type = genomic RNA
tacgcaacatccttacagcc

SEQ ID NO: 64 - DNA, 19 bp, Prunus dulcis
  source 1..19: mol_type = genomic DNA
aaccaaggaggaattgctt

SEQ ID NO: 65 - DNA, 20 bp, Prunus dulcis
  source 1..20: mol_type = genomic DNA
caccgtttttcttgcagaac

SEQ ID NO: 66 - DNA, 20 bp, Prunus dulcis
  source 1..20: mol_type = genomic DNA
ttaaccgatgggttccatat

SEQ ID NO: 67 - DNA, 20 bp, Prunus dulcis
  source 1..20: mol_type = genomic DNA
aatgtcaaagggatgatgat

SEQ ID NO: 68 - DNA, 20 bp, Prunus dulcis
  source 1..20: mol_type = genomic DNA
caatgttacatttgaagaga

SEQ ID NO: 69 - DNA, 20 bp, Prunus dulcis
  source 1..20: mol_type = genomic DNA
aagagagctccaagttaagc

SEQ ID NO: 70 - DNA, 20 bp, Prunus dulcis
  source 1..20: mol_type = genomic DNA
cactccggggttaatgacta

SEQ ID NO: 71 - DNA, 20 bp, Prunus dulcis
  source 1..20: mol_type = genomic DNA
tggaggttatgcatccacgt

SEQ ID NO: 72 - DNA, 20 bp, Prunus dulcis
  source 1..20: mol_type = genomic DNA
tggatattatagttgtgagg

SEQ ID NO: 73 - DNA, 20 bp, Prunus dulcis
  source 1..20: mol_type = genomic DNA
ctccttgtaaacgtggatgc

SEQ ID NO: 74 - DNA, 20 bp, Prunus dulcis
  source 1..20: mol_type = genomic DNA
ggtttgtacgcaacatcctt

SEQ ID NO: 75 - DNA, 20 bp, Prunus dulcis
  source 1..20: mol_type = genomic DNA
gtttgtacgcaacatcctta

SEQ ID NO: 76 - DNA, 20 bp, Prunus dulcis
  source 1..20: mol_type = genomic DNA
tacgcaacatccttacagcc

SEQ ID NO: 77 - RNA, 20 nt, Prunus dulcis
  source 1..20: mol_type = genomic RNA
tgcaacaatggccaccgacc

SEQ ID NO: 78 - RNA, 20 nt, Prunus dulcis
  source 1..20: mol_type = genomic RNA
aaaacgcttcaatcgtacca

SEQ ID NO: 79 - RNA, 20 nt, Prunus dulcis
  source 1..20: mol_type = genomic RNA
gaacagactcttggaagatg

SEQ ID NO: 80 - RNA, 20 nt, Prunus dulcis
  source 1..20: mol_type = genomic RNA
tcaaacccaacgaagcccag

SEQ ID NO: 81 - RNA, 20 nt, Prunus dulcis
  source 1..20: mol_type = genomic RNA
gtcccattgcaattactggg

SEQ ID NO: 82 - RNA, 20 nt, Prunus dulcis
  source 1..20: mol_type = genomic RNA
atgcaaggaaagtggtacgt

SEQ ID NO: 83 - RNA, 20 nt, Prunus dulcis
  source 1..20: mol_type = genomic RNA
aggatactgagacatcgaat

SEQ ID NO: 84 - RNA, 20 nt, Prunus dulcis
  source 1..20: mol_type = genomic RNA
tatcattgccactttccacg

SEQ ID NO: 85 - RNA, 20 nt, Prunus dulcis
  source 1..20: mol_type = genomic RNA
gtatttcctaagtatgggga

SEQ ID NO: 86 - RNA, 20 nt, Prunus dulcis
  source 1..20: mol_type = genomic RNA
ctttcttcttttgttacgtt

SEQ ID NO: 87 - RNA, 20 nt, Prunus dulcis
  source 1..20: mol_type = genomic RNA
ttcttgctttagtatttcct

SEQ ID NO: 88 - RNA, 20 nt, Prunus dulcis
  source 1..20: mol_type = genomic RNA
cttgttcttggttttgcttt

SEQ ID NO: 89 - RNA, 20 nt, Prunus dulcis
  source 1..20: mol_type = genomic RNA
acaatccatggcctatggcc

SEQ ID NO: 90 - RNA, 20 nt, Prunus dulcis
  source 1..20: mol_type = genomic RNA
cttgcatcaaatttagtccc

SEQ ID NO: 91 - RNA, 20 nt, Prunus dulcis
  source 1..20: mol_type = genomic RNA
ttccattcgtcttcccaaaa

SEQ ID NO: 92 - RNA, 20 nt, Prunus dulcis
  source 1..20: mol_type = genomic RNA
ggaagacgaatggaacaaac

SEQ ID NO: 93 - RNA, 20 nt, Prunus dulcis
  source 1..20: mol_type = genomic RNA
aggatctctgtaatattgta

SEQ ID NO: 94 - RNA, 20 nt, Prunus dulcis
  source 1..20: mol_type = genomic RNA
ttgctttatagccataacaa

SEQ ID NO: 95 - RNA, 20 nt, Prunus dulcis
  source 1..20: mol_type = genomic RNA
catcctggccgattacagtc

SEQ ID NO: 96 - RNA, 20 nt, Prunus dulcis
  source 1..20: mol_type = genomic RNA
tttttgcatcctggccgatt

SEQ ID NO: 97 - RNA, 20 nt, Prunus dulcis
  source 1..20: mol_type = genomic RNA
ttgaaacttgatgtcaattt

SEQ ID NO: 98 - DNA, 20 bp, Prunus dulcis
  source 1..20: mol_type = genomic DNA
tgcaacaatggccaccgacc

SEQ ID NO: 99 - DNA, 20 bp, Prunus dulcis
  source 1..20: mol_type = genomic DNA
aaaacgcttcaatcgtacca

SEQ ID NO: 100 - DNA, 20 bp, Prunus dulcis
  source 1..20: mol_type = genomic DNA
gaacagactcttggaagatg

SEQ ID NO: 101 - DNA, 20 bp, Prunus dulcis
  source 1..20: mol_type = genomic DNA
tcaaacccaacgaagcccag

SEQ ID NO: 102 - DNA, 20 bp, Prunus dulcis
  source 1..20: mol_type = genomic DNA
gtcccattgcaattactggg

SEQ ID NO: 103 - DNA, 20 bp, Prunus dulcis
  source 1..20: mol_type = genomic DNA
atgcaaggaaagtggtacgt

SEQ ID NO: 104 - DNA, 20 bp, Prunus dulcis
  source 1..20: mol_type = genomic DNA
aggatactgagacatcgaat

SEQ ID NO: 105 - DNA, 20 bp, Prunus dulcis
  source 1..20: mol_type = genomic DNA
tatcattgccactttccacg

SEQ ID NO: 106 - DNA, 20 bp, Prunus dulcis
  source 1..20: mol_type = genomic DNA
gtatttcctaagtatgggga

SEQ ID NO: 107 - DNA, 20 bp, Prunus dulcis
  source 1..20: mol_type = genomic DNA
ctttcttcttttgttacgtt

SEQ ID NO: 108 - DNA, 20 bp, Prunus dulcis
  source 1..20: mol_type = genomic DNA
ttcttgctttagtatttcct

SEQ ID NO: 109 - DNA, 20 bp, Prunus dulcis
  source 1..20: mol_type = genomic DNA
cttgttcttggttttgcttt

SEQ ID NO: 110 - DNA, 20 bp, Prunus dulcis
  source 1..20: mol_type = genomic DNA
acaatccatggcctatggcc

SEQ ID NO: 111 - DNA, 20 bp, Prunus dulcis
  source 1..20: mol_type = genomic DNA
cttgcatcaaatttagtccc

SEQ ID NO: 112 - DNA, 20 bp, Prunus dulcis
  source 1..20: mol_type = genomic DNA
ttccattcgtcttcccaaaa

SEQ ID NO: 113 - DNA, 20 bp, Prunus dulcis
  source 1..20: mol_type = genomic DNA
ggaagacgaatggaacaaac

SEQ ID NO: 114 - DNA, 20 bp, Prunus dulcis
  source 1..20: mol_type = genomic DNA
aggatctctgtaatattgta

SEQ ID NO: 115 - DNA, 20 bp, Prunus dulcis
  source 1..20: mol_type = genomic DNA
ttgctttatagccataacaa

SEQ ID NO: 116 - DNA, 20 bp, Prunus dulcis
  source 1..20: mol_type = genomic DNA
catcctggccgattacagtc

SEQ ID NO: 117 - DNA, 20 bp, Prunus dulcis
  source 1..20: mol_type = genomic DNA
tttttgcatcctggccgatt

SEQ ID NO: 118 - DNA, 20 bp, Prunus dulcis
  source 1..20: mol_type = genomic DNA
ttgaaacttgatgtcaattt

SEQ ID NO: 119 - RNA, 20 nt, Prunus dulcis
  source 1..20: mol_type = genomic RNA
ctaaccgatgggttccatat

SEQ ID NO: 120 - RNA, 20 nt, Prunus dulcis
  source 1..20: mol_type = genomic RNA
caagagtctgttctaagact

SEQ ID NO: 121 - RNA, 20 nt, Prunus dulcis
  source 1..20: mol_type = genomic RNA
aacgtcaaaatgacaacgat

SEQ ID NO: 122 - RNA, 20 nt, Prunus dulcis
  source 1..20: mol_type = genomic RNA
ggacgagatattgaattatg

SEQ ID NO: 123 - RNA, 20 nt, Prunus dulcis
  source 1..20: mol_type = genomic RNA
ggacccctccaataagcccc

SEQ ID NO: 124 - RNA, 20 nt, Prunus dulcis
  source 1..20: mol_type = genomic RNA
aatggagcggcataccacat

SEQ ID NO: 125 - RNA, 20 nt, Prunus dulcis
  source 1..20: mol_type = genomic RNA
atggagcggcataccacatc

SEQ ID NO: 126 - RNA, 20 nt, Prunus dulcis
  source 1..20: mol_type = genomic RNA
gtccttgtaagcgtcgatac

SEQ ID NO: 127 - RNA, 20 nt, Prunus dulcis
  source 1..20: mol_type = genomic RNA
aatcatcttccaagagtctg

SEQ ID NO: 128 - RNA, 20 nt, Prunus dulcis
  source 1..20: mol_type = genomic RNA
ttattccttggaaatattgc

SEQ ID NO: 129 - RNA, 20 nt, Prunus dulcis
  source 1..20: mol_type = genomic RNA
caaggaataataaaaggaca

SEQ ID NO: 130 - RNA, 20 nt, Prunus dulcis
  source 1..20: mol_type = genomic RNA
tggttgtgaggaggagggca

SEQ ID NO: 131 - RNA, 20 nt, Prunus dulcis
  source 1..20: mol_type = genomic RNA
agattttatggttgtgagga

SEQ ID NO: 132 - RNA, 20 nt, Prunus dulcis
  source 1..20: mol_type = genomic RNA
catttcggacgagatattga

SEQ ID NO: 133 - RNA, 20 nt, Prunus dulcis
  source 1..20: mol_type = genomic RNA
tacatgcaagtcatggagtg

SEQ ID NO: 134 - RNA, 20 nt, Prunus dulcis
  source 1..20: mol_type = genomic RNA
tttgtacatgcaagtcatgg

SEQ ID NO: 135 - DNA, 20 bp, Prunus dulcis
  source 1..20: mol_type = genomic DNA
ctaaccgatgggttccatat

SEQ ID NO: 136 - DNA, 20 bp, Prunus dulcis
  source 1..20: mol_type = genomic DNA
caagagtctgttctaagact

SEQ ID NO: 137 - DNA, 20 bp, Prunus dulcis
  source 1..20: mol_type = genomic DNA
aacgtcaaaatgacaacgat

SEQ ID NO: 138 - DNA, 20 bp, Prunus dulcis
  source 1..20: mol_type = genomic DNA
ggacgagatattgaattatg

SEQ ID NO: 139 - DNA, 20 bp, Prunus dulcis
  source 1..20: mol_type = genomic DNA
ggacccctccaataagcccc

SEQ ID NO: 140 - DNA, 20 bp, Prunus dulcis
  source 1..20: mol_type = genomic DNA
aatggagcggcataccacat

SEQ ID NO: 141 - DNA, 20 bp, Prunus dulcis
  source 1..20: mol_type = genomic DNA
atggagcggcataccacatc

SEQ ID NO: 142 - DNA, 20 bp, Prunus dulcis
  source 1..20: mol_type = genomic DNA
gtccttgtaagcgtcgatac

SEQ ID NO: 143 - DNA, 20 bp, Prunus dulcis
  source 1..20: mol_type = genomic DNA
aatcatcttccaagagtctg

SEQ ID NO: 144 - DNA, 20 bp, Prunus dulcis
  source 1..20: mol_type = genomic DNA
ttattccttggaaatattgc

SEQ ID NO: 145 - DNA, 20 bp, Prunus dulcis
  source 1..20: mol_type = genomic DNA
caaggaataataaaaggaca

SEQ ID NO: 146 - DNA, 20 bp, Prunus dulcis
  source 1..20: mol_type = genomic DNA
tggttgtgaggaggagggca

SEQ ID NO: 147 - DNA, 20 bp, Prunus dulcis
  source 1..20: mol_type = genomic DNA
agattttatggttgtgagga

SEQ ID NO: 148 - DNA, 20 bp, Prunus dulcis
  source 1..20: mol_type = genomic DNA
catttcggacgagatattga

SEQ ID NO: 149 - DNA, 20 bp, Prunus dulcis
  source 1..20: mol_type = genomic DNA
tacatgcaagtcatggagtg

SEQ ID NO: 150 - DNA, 20 bp, Prunus dulcis
  source 1..20: mol_type = genomic DNA
tttgtacatgcaagtcatgg

SEQ ID NO: 151 - DNA, 100 bp, Prunus dulcis
  source 1..100: mol_type = genomic DNA
cccatgcaacctcctatataaacagcaatacaagtgaatcttagctgagaaagcctctcccattctgttgtatttcttattgttttagtattttctaagt

SEQ ID NO: 152 - DNA, 100 bp, Prunus dulcis
  source 1..100: mol_type = genomic DNA
atgcaccctcctatataagcagcaaacacattgagtcgtcgtagctcagaaagcctctatcattctgttgtatttcttcttgctttagtatttcctaagt

SEQ ID NO: 153 - DNA, 1125 bp, Prunus dulcis; identical to SEQ ID NO: 9
  source 1..1125: mol_type = genomic DNA

SEQ ID NO: 154 - DNA, 752 bp, Prunus dulcis
  source 1..752: mol_type = genomic DNA
agaaagcctctatcattctgttgtatttcttcttgctttagtatttcctaagtatggggatgttgaaatcgtcactcgctttccttgttcttggttttgctttcttcttttgttacgttatgagcagtggatcttatgactattttcaatttgtgcaacaatggccaccgaccaactgcagagttcgcatgaagcgaccttgctccaatccccggccattacaatatttcacaatccatggcctatggccaagtaatttttcaaacccaacgaagcccagtaattgcaatgggactaaatttgatgcaaggaaagtgtatcctgaaatgcgatccgatctgaagatatcttggccagacgtggaaagtggcaatgatacaaaattttgggaagacgaatggaacaaacacggtacatgttctgaacagacacttaatcaattccagtactttgagcgatcccacgaaatgtggatgtcgtacaatattacagagatccttaaaaacgcttcaatcgtaccacatccggcaaaaacatggacctattcggacatagtatcacccatcaaagcagcaactggaagaacaccgcttcttcgttgcaaatacgataacaacactcagttgttacatgaagtggtattttgttatggctataaagcaataaagcagattgactgtaatcggccaggatgcaaaaataaaattgacatcaagtttcaataaaataatagctagctttctaaaatcgta

SEQ ID NO: 155 - DNA, 1131 bp, Prunus dulcis; identical to SEQ ID NO: 4
  source 1..1131: mol_type = genomic DNA

SEQ ID NO: 156 - DNA, 684 bp, Prunus dulcis; identical to SEQ ID NO: 2
  source 1..684: mol_type = genomic DNA

SEQ ID NO: 157 - protein, 374 aa, Prunus dulcis; identical to SEQ ID NO: 10

SEQ ID NO: 158 - protein, 223 aa, Prunus dulcis; identical to SEQ ID NO: 8

SEQ ID NO: 159 - protein, 376 aa, Prunus dulcis; identical to SEQ ID NO: 5

SEQ ID NO: 160 - protein, 227 aa, Prunus dulcis; identical to SEQ ID NO: 3

SEQ ID NO: 161 - DNA, 1113 bp, Prunus dulcis
  source 1..1113: mol_type = genomic DNA
atgacattcgcactacgtaagaaagaaatcttaatcgacatcctagtaagactacctgcaaaatcccttgttcgatttctgtgtacatgcaagtcatggagtgatttgattggcagctcgagttttgttggcacacaccttcataggaatgtcacgggacatgcccaggcctatctcctttgcctccaccacccaaattttgaatgtcaaagggatgatgatgacccatattttaaagaagaactccaatggtcactgttttccaatgttacatttgaagagagctccaagttaagccatcccttagggagcacagaacattatgtgatatatggttcaagcaatggtttagtttgcatatcggatgagataatgaatttcgatagtcctatacacatatggaacccatcggttaagaaacttagaaccactccaatcagcaccagcattaacattaaatttagccatgttgctctccaattcggtttccactccggggttaagatgttgcgtacaaaccaaaatgccttggcggttgagatttatagtctcagaacagactcttggacgatgattgaagcaattcctccttggttaaaatgcacttggcagcatcatcagggtacattttttaatggagtagcataccacatcattgagaaaggtcctacattcagtgttatatcattcgattcaggcagtgaagaatttgaagaattcatagcaccagatgccatttgcagtttatggaggttatgcatccacgtttacaaggagcaaatttgcttgctttttggatattatagttgtgaggaggagggcatggaaaacattgacttatgggttctgcaagaaaaacggtggaaacaattgtgtccttttatttatgatcccttggatgatttctatcagataatcgggattagtactgacaataaactcataatggcaagaaaagatttcagtaagggcggagtagaactgcaattggggaattacgaatccaaacaagttcttgaaacagggattaagttggccatcatgagatatggcgaaagcgaattcttgtttgcaatgacttacgtagaaagtttagctttactcaataactag

SEQ ID NO: 162 - DNA, 740 bp, Prunus dulcis
  source 1..740: mol_type = genomic DNA
agaaagcctctatcattctgttgtatttcttcttgctttagtatttcctaagtaaatcgtcactcgctttccttgttcttggttttgctttcttcttttgttacgttatgagcagtggatcttatgactattttcaatttgtgcaacaatggccaccgaccaactgcagagttcgcatgaagcgaccttgctccaatccccggccattacaatatttcacaatccatggcctatggccaagtaatttttcaaacccaacgaagcccagtaattgcaatgggactaaatttgatgcaaggaaagtgtatcctgaaatgcgatccgatctgaagatatcttggccagacgtggaaagtggcaatgatacaaaattttgggaagacgaatggaacaaacacggtacatgttctgaacagacacttaatcaattccagtactttgagcgatcccacgaaatgtggatgtcgtacaatattacagagatccttaaaaacgcttcaatcgtaccacatccggcaaaaacatggacctattcggacatagtatcacccatcaaagcagcaactggaagaacaccgcttcttcgttgcaaatacgataacaacactcagttgttacatgaagtggtattttgttatggctataaagcaataaagcagattgactgtaatcggccaggatgcaaaaataaaattgacatcaagtttcaataaaataatagctagctttctaaaatcgta

SEQ ID NO: 163 - DNA, 675 bp, Prunus dulcis
  source 1..675: mol_type = genomic DNA
atggcgacgtcattcgctttccttgttctagcttttgctttcttcttgtgtttcattatgagcactggatcttacgtgtattttcaatttgtgcaacaatggccaccgacgacctgcagacttagcagcaaaccttccaaccaacaccggccattacaaagattcaccatccatggcctatggccaagtaactattcaaacccaaggaagcccagtaattgcaatgggtcacaatttaactttatgaaagtgtatcctcagttgcgaaccaaactgaagagatcttggccggacgtggaaggtggcaatgatacaaagttttgggaaggcgaatggaacaagcatggcacatgttccgaacggacgcttaaccaaatgcaatacttcgaggtatcccacgccatgtggaggtcgtacaatattacaaatatccttaaagacgctcatatcgtaccaaatccaacacaaagatggaagtactcggacatagtatcacccattaaaacagcaactggaagaacacccacccttcgttgcaaaactgatccagcaatgcctaacaactctcagttgttacatgaagtggtattttgttatggatataatgcaaaactgcatattgactgtaatcgaacagcaggatgccgaaatcatattgacatcttgtttcaataa

SEQ ID NO: 164 - DNA, 1123 bp, Prunus dulcis
  source 1..1123: mol_type = genomic DNA
atgacattcgcactacgtaagaaagaaatcttaatcgacatcctagtaagactacctgcaaaatcccttgttcgatttctgtgtacatgcaagtcatggagtgatttgattggcagctcgagttttgttggcacacaccttcataggaatgtcacgggacatgcccaggcctatctcctttgcctccaccacccaaattttgaatgtcaaagggatgatgatgacccatattttaaagaagaactccaatggtcactgttttccaatgttacatttgaagagagctccaagttaagccatcccttagggagcacagaacattatgtgatatatggttcaagcaatggtttagtttgcatatcggatgagataatgaatttcgatagtcctatacacatatggaacccatcggttaagaaacttagaaccactccaatcagcaccagcattaacattaaatttagccatgttgctctccaattcggtttccactccggggttaatgactgtaaggatgttgcgtacaaaccaaaatgccttggcggttgagatttatagtctcagaacagactcttggacgatgattgaagcaattcctccttggttaaaatgcacttggcagcatcatcagggtacattttttaatggagtagcataccacatcattgagaaaggtcctacattcagtgttatatcattcgattcaggcagtgaagaatttgaagaattcatagcaccagatgccatttgcagtttatggaggttatgcatccacgtttacaaggagcaaatttgcttgctttttggatattatagttgtgaggaggagggcatggaaaacattgacttatgggttctgcaagaaaaacggtggaaacaattgtgtccttttatttatgatcccttggatgatttctatcagataatcgggattagtactgacaataaactcataatggcaagaaaagatttcagtaagggcggagtagaactgcaattggggaattacgaatccaaacaagttcttgaaacagggattaagttggccatcatgagatatggcgaaagcgaattcttgtttgcaatgacttacgtagaaagtttagctttactcaataactag

SEQ ID NO: 165 - DNA, 674 bp, Prunus dulcis
  source 1..674: mol_type = genomic DNA
agagacagtcattcgctttccttgttctagcttttgctttcttcttgtgtttcattatgagcactggatcttacgtgtattttcaatttgtgcaacaatggccaccgacgacctgcagacttagcagcaaaccttccaaccaacaccggccattacaaagattcaccatccatggcctatggccaagtaactattcaaacccaaggaagcccagtaattgcaatgggtcacaatttaactttatgaaagtgtatcctcagttgcgaaccaaactgaagagatcttggccggacgtggaaggtggcaatgatacaaagttttgggaaggcgaatggaacaagcatggcacatgttccgaacggacgcttaaccaaatgcaatacttcgaggtatcccacgccatgtggaggtcgtacaatattacaaatatccttaaagacgctcatatcgtaccaaatccaacacaaagatggaagtactcggacatagtatcacccattaaaacagcaactggaagaacacccacccttcgttgcaaaactgatccagcaatgcctaacaactctcagttgttacatgaagtggtattttgttatggatataatgcaaaactgcatattgactgtaatcgaacagcaggatgccgaaatcatattgacatcttgtttcaataa

SEQ ID NO: 166 - DNA, 1206 bp, Prunus dulcis
  source 1..1206: mol_type = genomic DNA
atgacattcaaattaggtaagaaagaaatcttgatcgacatcctagcaagactaactgcaaaatccctggttcggtttctttgtacatgcaagtcatggagtgatttgattggcaggtccagttttgtcagcacacacattaataggaatgtcactaaacatgcccacgtctatctactttgcctccaccacccaacttttgaacgtcaaaatgacaacgatgacccatttgttgaagaagaacttctatggtcacttttttcaaatgaaacatttgagcagttctctaagttaagcaatcccttaggaagcacagaacattatgggatatatggttcaagcaatggtctagtttgcatttcggacgagatattgaattatgatagtcctatacacatatggaacccatcggttaggaaatttaggacccctccaataagccaacaacattaaatttgtctacgttgctctccaattcggcttccactcgggggttaatgactacaaggctgtaagaatgatgcgttccaacaaagataccttcgcggttgaggtttatagtcttagaacagactcttggaagatgattgaaacaattcctccttggttaaaatgcacttggcagcatcatacgggtacattttttaatggagcggcataccacatcattgagaaaggtcccttattcagcattatgtccttcgattcaggcagtgaagagttcgaagaattcattgcaccagatgccatttgcaattcacgggggttgtgtatcgacgcttacaaggaccaaatttgcttgcttttcagattttatggttgtgaggaggagggcatgcgaaaagttgacttgtgggttctgcaagaaaaacgatggaaacaattgtgtccttttattattccttggaaatattgccgtacaattgggactagtatagataatgagctcttattgcaaaaaaaagatttcaataagggcgcagcagatctatgtttgtgtaattacgaatccaagcaagttcttgaaacaggaattaagttggccatcatgacatatggcgaaatcgatttcttgttttcatttacttacatagaaagtttggttttactcaataattattaaacagatttaaactctgtttttattcggtttcttttgtaagggtaattattgaagggagatttaagagaaaaacattccatgtt

SEQ ID NO: 167 - DNA, 1123 bp, Prunus dulcis
  source 1..1123: mol_type = genomic DNA
atgacattcgcactacgtaagaaagaaatcttaatcgacatcctagtaagactacctgcaaaatcccttgttcgatttctgtgtacatgcaagtcatggagtgatttgattggcagctcgagttttgttggcacacaccttcataggaatgtcacgggacatgcccaggcctatctcctttgcctccaccacccaaattttgaatgtcaaagggatgatgatgacccatattttaaagaagaactccaatggtcactgttttccaatgttacatttgaagagagctccaagttaagccatcccttagggagcacagaacattatgtgatatatggttcaagcaatggtttagtttgcatatcggatgagataatgaatttcgatagtcctatacacatatggaacccatcggttaagaaacttagaaccactccaatcagcaccagcattaacattaaatttagccatgttgctctccaattcggtttccactccggggttaagggctgtaaggatgttgcgtacaaaccaaaatgccttggcggttgagatttatagtctcagaacagactcttggacgatgattgaagcaattcctccttggttaaaatgcacttggcagcatcatcagggtacattttttaatggagtagcataccacatcattgagaaaggtcctacattcagtgttatatcattcgattcaggcagtgaagaatttgaagaattcatagcaccagatgccatttgcagtttatggaggttatgcatccacgtttacaaggagcaaatttgcttgctttttggatattatagttgtgaggaggagggcatggaaaacattgacttatgggttctgcaagaaaaacggtggaaacaattgtgtccttttatttatgatcccttggatgatttctatcagataatcgggattagtactgacaataaactcataatggcaagaaaagatttcagtaagggcggagtagaactgcaattggggaattacgaatccaaacaagttcttgaaacagggattaagttggccatcatgagatatggcgaaagcgaattcttgtttgcaatgacttacgtagaaagtttagctttactcaataactag

SEQ ID NO: 168 - DNA, 675 bp, Prunus dulcis; identical to SEQ ID NO: 163
  source 1..675: mol_type = genomic DNA

SEQ ID NO: 169 - DNA, 1194 bp, Prunus dulcis
  source 1..1194: mol_type = genomic DNA
atgacattcaaattaggtaagaaagaaatcttgatcgacatcctagcaagactaactgcaaaatccctggttcggtttctttgtacatgcaagtcatggagtgatttgattggcaggtccagttttgtcagcacacacattaataggaatgtcactaaacatgcccacgtctatctactttgcctccaccacccaacttttgaacgtcaaaatgacaacgatgacccatttgttgaagaagaacttctatggtcacttttttcaaatgaaacatttgagcagttctctaagttaagcaatcccttaggaagcacagaacattatgggatatatggttcaagcaatggtctagtttgcatttcggacgagatattgaattatgatagtcctatacacatatggaacccatcggttaggaaatttaggacccctccaataagccccaacaacattaaatttgtctacgttgctctccaattcggcttccactcgggggttaatgactacaaggctgtaagaatgatgcgttccaacaaagataccttcgcggttgaggtttatagtcttagaacagactcttggaagatgattgaaacaattcctccttggttaaaatgcacttggcagcatcatacgggtacattttttaatggagcggcagaaaggtcccttattcagcattatgtccttcgattcaggcagtgaagagttcgaagaattcattgcaccagatgccatttgcaattcacgggggttgtgtatcgacgcttacaaggaccaaatttgcttgcttttcagattttatggttgtgaggaggagggcatgcgaaaagttgacttgtgggttctgcaagaaaaacgatggaaacaattgtgtccttttattattccttggaaatattgccgtacaattgggactagtatagataatgagctcttattgcaaaaaaaagatttcaataagggcgcagcagatctatgtttgtgtaattacgaatccaagcaagttcttgaaacaggaattaagttggccatcatgacatatggcgaaatcgatttcttgttttcatttacttacatagaaagtttggttttactcaataattattaaacagatttaaactctgtttttattcggtttcttttgtaagggtaattattgaagggagatttaagagaaaaacattccatgtt

SEQ ID NO: 170 - DNA, 744 bp, Prunus dulcis
  source 1..744: mol_type = genomic DNA
agaaagcctctatcattctgttgtatttcttcttgctttagtatttcctaagtatggggatgttgaaatcgtcactcgctttccttgttcttggttttgctttcttcttttgttacgttatgagcagtggatcttatgactattttcaatttgtgcaacaatggccaccgaccaactgcagagttcgcatgaagcgaccttgctccaatccccggccattacaatatttcacaatccatggcctgtaatttttcaaacccaacgaagcccagtaattgcaatgggactaaatttgatgcaaggaaagtgtatcctgaaatgcgatccgatctgaagatatcttggccagacgtggaaagtggcaatgatacaaaattttgggaagacgaatggaacaaacacggtacatgttctgaacagacacttaatcaattccagtactttgagcgatcccacgaaatgtggatgtcgtacaatattacagagatccttaaaaacgcttcaatcgtaccacatccggcaaaaacatggacctattcggacatagtatcacccatcaaagcagcaactggaagaacaccgcttcttcgttgcaaatacgataacaacactcagttgttacatgaagtggtattttgttatggctataaagcaataaagcagattgactgtaatcggccaggatgcaaaaataaaattgacatcaagtttcaataaaataatagctagctttctaaaatcgta

SEQ ID NO: 171 - DNA, 1124 bp, Prunus dulcis
  source 1..1124: mol_type = genomic DNA
atgacattcgcactacgtaagaaagaaatcttaatcgacatcctagtaagactacctgcaaaatcccttgttcgatttctgtgtacatgcaagtcatggagtgatttgattggcagctcgagttttgttggcacacaccttcataggaatgtcacgggacatgcccaggcctatctcctttgcctccaccacccaaattttgaatgtcaaagggatgatgatgacccatattttaaagaagaactccaatggtcactgttttccaatgttacatttgaagagagctccaagttaagccatcccttagggagcacagaacattatgtgatatatggttcaagcaatggtttagtttgcatatcggatgagataatgaatttcgatagtcctatacacatatggaacccatcggttaagaaacttagaaccactccaatcagcaccagcattaacattaaatttagccatgttgctctccaattcggtttccactccggggttaatgggctgtaaggatgttgcgtacaaaccaaaatgccttggcggttgagatttatagtctcagaacagactcttggacgatgattgaagcaattcctccttggttaaaatgcacttggcagcatcatcagggtacattttttaatggagtagcataccacatcattgagaaaggtcctacattcagtgttatatcattcgattcaggcagtgaagaatttgaagaattcatagcaccagatgccatttgcagtttatggaggttatgcatccacgtttacaaggagcaaatttgcttgctttttggatattatagttgtgaggaggagggcatggaaaacattgacttatgggttctgcaagaaaaacggtggaaacaattgtgtccttttatttatgatcccttggatgatttctatcagataatcgggattagtactgacaataaactcataatggcaagaaaagatttcagtaagggcggagtagaactgcaattggggaattacgaatccaaacaagttcttgaaacagggattaagttggccatcatgagatatggcgaaagcgaattcttgtttgcaatgacttacgtagaaagtttagctttactcaataactag

SEQ ID NO: 172 - DNA, 680 bp, Prunus dulcis
  source 1..680: mol_type = genomic DNA
atggcgacgttgagtcattcgctttccttgttctagcttttgctttcttcttgtgtttcattatgagcactggatcttacgtgtattttcaatttgtgcaacaatggccaccgacgacctgcagacttagcagcaaaccttccaaccaacaccggccattacaaagattcaccatccatggcctatggccaagtaactattcaaacccaaggaagcccagtaattgcaatgggtcacaatttaactttatgaaagtgtatcctcagttgcgaaccaaactgaagagatcttggccggacgtggaaggtggcaatgatacaaagttttgggaaggcgaatggaacaagcatggcacatgttccgaacggacgcttaaccaaatgcaatacttcgaggtatcccacgccatgtggaggtcgtacaatattacaaatatccttaaagacgctcatatcgtaccaaatccaacacaaagatggaagtactcggacatagtatcacccattaaaacagcaactggaagaacacccacccttcgttgcaaaactgatccagcaatgcctaacaactctcagttgttacatgaagtggtattttgttatggatataatgcaaaactgcatattgactgtaatcgaacagcaggatgccgaaatcatattgacatcttgtttcaataa

SEQ ID NO: 173 - DNA, 1119 bp, Prunus dulcis
  source 1..1119: mol_type = genomic DNA
atgacattcaaattaggtaagaaagaaatcttgatcgacatcctagcaagactaactgcaaaatccctggttcggtttctttgtacatgcaagtcatggagtgatttgattggcaggtccagttttgtcagcacacacattaataggaatgtcactaaacatgcccacgtctatctactttgcctccaccacccaacttttgaacgtcaaaatgacaacgatgacccatttgttgaagaagaacttctatggtcacttttttcaaatgaaacatttgagcagttctctaagttaagcaatcccttaggaagcacagaacattatgggatatatggttcaagcaatggtctagtttgcatttcggacgagatattgaattatgatagtcctatacacatatggaacccatcggttaggaaatttaggacccctccaataagccccaacaacattaaatttgtctacgttgctctccaattcggcttccactcgggggttaatgactacaaggctgtaagaatgatgcgttccaacaaagataccttcgcggttgaggtttatagtcttagaacagactcttggaagatgattgaaacaattcctccttggttaaaatgcacttggcagcatcatacgggtacattttttaatggagcggcataccttgagaaaggtcccttattcagcattatgtccttcgattcaggcagtgaagagttcgaagaattcattgcaccagatgccatttgcaattcacgggggttgtgtatcgacgcttacaaggaccaaatttgcttgcttttcagattttatggttgtgaggaggagggcatgcgaaaagttgacttgtgggttctgcaagaaaaacgatggaaacaattgtgtccttttattattccttggaaatattgccgtacaattgggactagtatagataatgagctcttattgcaaaaaaaagatttcaataagggcgcagcagatctatgtttgtgtaattacgaatccaagcaagttcttgaaacaggaattaagttggccatcatgacatatggcgaaatcgatttcttgttttcatttacttacatagaaagtttggttttactcaataattattaa

SEQ ID NO: 174 - DNA, 1123 bp, Prunus dulcis; identical to SEQ ID NO: 167
  source 1..1123: mol_type = genomic DNA

SEQ ID NO: 175 - DNA, 1124 bp, Prunus dulcis; identical to SEQ ID NO: 171
  source 1..1124: mol_type = genomic DNA

SEQ ID NO: 176 - DNA, 1123 bp, Prunus dulcis
  source 1..1123: mol_type = genomic DNA
atgacattcaaattaggtaagaaagaaatcttgatcgacatcctagcaagactaactgcaaaatccctggttcggtttctttgtacatgcaagtcatggagtgatttgattggcaggtccagttttgtcagcacacacattaataggaatgtcactaaacatgcccacgtctatctactttgcctccaccacccaacttttgaacgtcaaaatgacaacgatgacccatttgttgaagaagaacttctatggtcacttttttcaaatgaaacatttgagcagttctctaagttaagcaatcccttaggaagcacagaacattatgggatatatggttcaagcaatggtctagtttgcatttcggacgagatattgaattatgatagtcctatacacatatggaacccatcggttaggaaatttaggacccctccaataagccccaacaacattaaatttgtctacgttgctctccaattcggcttccactcgggggttaatgactacaaggctgtaagaatgatgcgttccaacaaagataccttcgcggttgaggtttatagtcttagaacagactcttggaagatgattgaaacaattcctccttggttaaaatgcacttggcagcatcatacgggtacattttttaatggagcggcataccatcattgagaaaggtcccttattcagcattatgtccttcgattcaggcagtgaagagttcgaagaattcattgcaccagatgccatttgcaattcacgggggttgtgtatcgacgcttacaaggaccaaatttgcttgcttttcagattttatggttgtgaggaggagggcatgcgaaaagttgacttgtgggttctgcaagaaaaacgatggaaacaattgtgtccttttattattccttggaaatattgccgtacaattgggactagtatagataatgagctcttattgcaaaaaaaagatttcaataagggcgcagcagatctatgtttgtgtaattacgaatccaagcaagttcttgaaacaggaattaagttggccatcatgacatatggcgaaatcgatttcttgttttcatttacttacatagaaagtttggttttactcaataattattaa

SEQ ID NO: 177 - DNA, 725 bp, Prunus dulcis
  source 1..725: mol_type = genomic DNA
agaaagcctctatcattctgttgtatttcttcttgctttagtatttcctcgctttccttgttcttggttttgctttcttcttttgttacgttatgagcagtggatcttatgactattttcaatttgtgcaacaatggccaccgaccaactgcagagttcgcatgaagcgaccttgctccaatccccggccattacaatatttcacaatccatggcctatggccaagtaatttttcaaacccaacgaagcccagtaattgcaatgggactaaatttgatgcaaggaaagtgtatcctgaaatgcgatccgatctgaagatatcttggccagacgtggaaagtggcaatgatacaaaattttgggaagacgaatggaacaaacacggtacatgttctgaacagacacttaatcaattccagtactttgagcgatcccacgaaatgtggatgtcgtacaatattacagagatccttaaaaacgcttcaatcgtaccacatccggcaaaaacatggacctattcggacatagtatcacccatcaaagcagcaactggaagaacaccgcttcttcgttgcaaatacgataacaacactcagttgttacatgaagtggtattttgttatggctataaagcaataaagcagattgactgtaatcggccaggatgcaaaaataaaattgacatcaagtttcaataaaataatagctagctttctaaaatcgta

SEQ ID NO: 178 - DNA, 739 bp, Prunus dulcis
  source 1..739: mol_type = genomic DNA
agaaagcctctatcattctgttgtatttcttcttgctttagtatttcctaagtaatcgtcactcgctttccttgttcttggttttgctttcttcttttgttacgttatgagcagtggatcttatgactattttcaatttgtgcaacaatggccaccgaccaactgcagagttcgcatgaagcgaccttgctccaatccccggccattacaatatttcacaatccatggcctatggccaagtaatttttcaaacccaacgaagcccagtaattgcaatgggactaaatttgatgcaaggaaagtgtatcctgaaatgcgatccgatctgaagatatcttggccagacgtggaaagtggcaatgatacaaaattttgggaagacgaatggaacaaacacggtacatgttctgaacagacacttaatcaattccagtactttgagcgatcccacgaaatgtggatgtcgtacaatattacagagatccttaaaaacgcttcaatcgtaccacatccggcaaaaacatggacctattcggacatagtatcacccatcaaagcagcaactggaagaacaccgcttcttcgttgcaaatacgataacaacactcagttgttacatgaagtggtattttgttatggctataaagcaataaagcagattgactgtaatcggccaggatgcaaaaataaaattgacatcaagtttcaataaaataatagctagctttctaaaatcgta

SEQ ID NO: 179 - DNA, 739 bp, Prunus dulcis; identical to SEQ ID NO: 178
  source 1..739: mol_type = genomic DNA

SEQ ID NO: 180 - DNA, 1120 bp, Prunus dulcis
  source 1..1120: mol_type = genomic DNA
atgacattcaaattaggtaagaaagaaatcttgatcgacatcctagcaagactaactgcaaaatccctggttcggtttctttgtacatgcaagtcatggagtgatttgattggcaggtccagttttgtcagcacacacattaataggaatgtcactaaacatgcccacgtctatctactttgcctccaccacccaacttttgaacgtcaaaatgacaacgatgacccatttgttgaagaagaacttctatggtcacttttttcaaatgaaacatttgagcagttctctaagttaagcaatcccttaggaagcacagaacattatgggatatatggttcaagcaatggtctagtttgcatttcggacgagatattgaattatgatagtcctatacacatatggaacccatcggttaggaaatttaggacccctccaataagccccaacaacattaaatttgtctacgttgctctccaattcggcttccactcgggggttaatgactacaaggctgtaagaatgatgcgttccaacaaagataccttcgcggttgaggtttatagtcttagaacagactcttggaagatgattgaaacaattcctccttggttaaaatgcacttggcagcatcatacgggtacattttttaatggagcggcataccattgagaaaggtcccttattcagcattatgtccttcgattcaggcagtgaagagttcgaagaattcattgcaccagatgccatttgcaattcacgggggttgtgtatcgacgcttacaaggaccaaatttgcttgcttttcagattttatggttgtgaggaggagggcatgcgaaaagttgacttgtgggttctgcaagaaaaacgatggaaacaattgtgtccttttattattccttggaaatattgccgtacaattgggactagtatagataatgagctcttattgcaaaaaaaagatttcaataagggcgcagcagatctatgtttgtgtaattacgaatccaagcaagttcttgaaacaggaattaagttggccatcatgacatatggcgaaatcgatttcttgttttcatttacttacatagaaagtttggttttactcaataattattaa

SEQ ID NO: 181 - DNA, 734 bp, Prunus dulcis
  source 1..734: mol_type = genomic DNA
agaaagcctctatcattctgttgtatttcttcttgctttagtatttcctaagtatggtcgctttccttgttcttggttttgctttcttcttttgttacgttatgagcagtggatcttatgactattttcaatttgtgcaacaatggccaccgaccaactgcagagttcgcatgaagcgaccttgctccaatccccggccattacaatatttcacaatccatggcctatggccaagtaatttttcaaacccaacgaagcccagtaattgcaatgggactaaatttgatgcaaggaaagtgtatcctgaaatgcgatccgatctgaagatatcttggccagacgtggaaagtggcaatgatacaaaattttgggaagacgaatggaacaaacacggtacatgttctgaacagacacttaatcaattccagtactttgagcgatcccacgaaatgtggatgtcgtacaatattacagagatccttaaaaacgcttcaatcgtaccacatccggcaaaaacatggacctattcggacatagtatcacccatcaaagcagcaactggaagaacaccgcttcttcgttgcaaatacgataacaacactcagttgttacatgaagtggtattttgttatggctataaagcaataaagcagattgactgtaatcggccaggatgcaaaaataaaattgacatcaagtttcaataaaataatagctagctttctaaaatcgta

SEQ ID NO: 182 - DNA, 677 bp, Prunus dulcis
  source 1..677: mol_type = genomic DNA
atttgagacagtcattcgctttccttgttctagcttttgctttcttcttgtgtttcattatgagcactggatcttacgtgtattttcaatttgtgcaacaatggccaccgacgacctgcagacttagcagcaaaccttccaaccaacaccggccattacaaagattcaccatccatggcctatggccaagtaactattcaaacccaaggaagcccagtaattgcaatgggtcacaatttaactttatgaaagtgtatcctcagttgcgaaccaaactgaagagatcttggccggacgtggaaggtggcaatgatacaaagttttgggaaggcgaatggaacaagcatggcacatgttccgaacggacgcttaaccaaatgcaatacttcgaggtatcccacgccatgtggaggtcgtacaatattacaaatatccttaaagacgctcatatcgtaccaaatccaacacaaagatggaagtactcggacatagtatcacccattaaaacagcaactggaagaacacccacccttcgttgcaaaactgatccagcaatgcctaacaactctcagttgttacatgaagtggtattttgttatggatataatgcaaaactgcatattgactgtaatcgaacagcaggatgccgaaatcatattgacatcttgtttcaataa

SEQ ID NO: 183 - DNA, 729 bp, Prunus dulcis
  source 1..729: mol_type = genomic DNA
agaaagcctctatcattctgttgtatttcttcttgctttagtatttcctaactcgctttccttgttcttggttttgctttcttcttttgttacgttatgagcagtggatcttatgactattttcaatttgtgcaacaatggccaccgaccaactgcagagttcgcatgaagcgaccttgctccaatccccggccattacaatatttcacaatccatggcctatggccaagtaatttttcaaacccaacgaagcccagtaattgcaatgggactaaatttgatgcaaggaaagtgtatcctgaaatgcgatccgatctgaagatatcttggccagacgtggaaagtggcaatgatacaaaattttgggaagacgaatggaacaaacacggtacatgttctgaacagacacttaatcaattccagtactttgagcgatcccacgaaatgtggatgtcgtacaatattacagagatccttaaaaacgcttcaatcgtaccacatccggcaaaaacatggacctattcggacatagtatcacccatcaaagcagcaactggaagaacaccgcttcttcgttgcaaatacgataacaacactcagttgttacatgaagtggtattttgttatggctataaagcaataaagcagattgactgtaatcggccaggatgcaaaaataaaattgacatcaagtttcaataaaataatagctagctttctaaaatcgta

SEQ ID NO: 184 - DNA, 670 bp, Prunus dulcis
  source 1..670: mol_type = genomic DNA
acagtcattcgctttccttgttctagcttttgctttcttcttgtgtttcattatgagcactggatcttacgtgtattttcaatttgtgcaacaatggccaccgacgacctgcagacttagcagcaaaccttccaaccaacaccggccattacaaagattcaccatccatggcctatggccaagtaactattcaaacccaaggaagcccagtaattgcaatgggtcacaatttaactttatgaaagtgtatcctcagttgcgaaccaaactgaagagatcttggccggacgtggaaggtggcaatgatacaaagttttgggaaggcgaatggaacaagcatggcacatgttccgaacggacgcttaaccaaatgcaatacttcgaggtatcccacgccatgtggaggtcgtacaatattacaaatatccttaaagacgctcatatcgtaccaaatccaacacaaagatggaagtactcggacatagtatcacccattaaaacagcaactggaagaacacccacccttcgttgcaaaactgatccagcaatgcctaacaactctcagttgttacatgaagtggtattttgttatggatataatgcaaaactgcatattgactgtaatcgaacagcaggatgccgaaatcatattgacatcttgtttcaataa

SEQ ID NO: 185 - DNA, 1117 bp, Prunus dulcis
  source 1..1117: mol_type = genomic DNA
atgacattcaaattaggtaagaaagaaatcttgatcgacatcctagcaagactaactgcaaaatccctggttcggtttctttgtacatgcaagtcatggagtgatttgattggcaggtccagttttgtcagcacacacattaataggaatgtcactaaacatgcccacgtctatctactttgcctccaccacccaacttttgaacgtcaaaatgacaacgatgacccatttgttgaagaagaacttctatggtcacttttttcaaatgaaacatttgagcagttctctaagttaagcaatcccttaggaagcacagaacattatgggatatatggttcaagcaatggtctagtttgcatttcggacgagatattgaattatgatagtcctatacacatatggaacccatcggttaggaaatttaggacccctccaataagccccaacaacattaaatttgtctacgttgctctccaattcggcttccactcgggggttaatgactacaaggctgtaagaatgatgcgttccaacaaagataccttcgcggttgaggtttatagtcttagaacagactcttggaagatgattgaaacaattcctccttggttaaaatgcacttggcagcatcatacgggtacattttttaatggagcggcatactgagaaaggtcccttattcagcattatgtccttcgattcaggcagtgaagagttcgaagaattcattgcaccagatgccatttgcaattcacgggggttgtgtatcgacgcttacaaggaccaaatttgcttgcttttcagattttatggttgtgaggaggagggcatgcgaaaagttgacttgtgggttctgcaagaaaaacgatggaaacaattgtgtccttttattattccttggaaatattgccgtacaattgggactagtatagataatgagctcttattgcaaaaaaaagatttcaataagggcgcagcagatctatgtttgtgtaattacgaatccaagcaagttcttgaaacaggaattaagttggccatcatgacatatggcgaaatcgatttcttgttttcatttacttacatagaaagtttggttttactcaataattattaa

SEQ ID NO: 186 - DNA, 1116 bp, Prunus dulcis
  source 1..1116: mol_type = genomic DNA
atgacattcgcactacgtaagaaagaaatcttaatcgacatcctagtaagactacctgcaaaatcccttgttcgatttctgtgtacatgcaagtcatggagtgatttgattggcagctcgagttttgttggcacacaccttcataggaatgtcacgggacatgcccaggcctatctcctttgcctccaccacccaaattttgaatgtcaaagggatgatgatgacccatattttaaagaagaactccaatggtcactgttttccaatgttacatttgaagagagctccaagttaagccatcccttagggagcacagaacattatgtgatatatggttcaagcaatggtttagtttgcatatcggatgagataatgaatttcgatagtcctatacacatatggaacccatcggttaagaaacttagaaccactccaatcagcaccagcattaacattaaatttagccatgttgctctccaattcggtttccactccggggttaatgggatgttgcgtacaaaccaaaatgccttggcggttgagatttatagtctcagaacagactcttggacgatgattgaagcaattcctccttggttaaaatgcacttggcagcatcatcagggtacattttttaatggagtagcataccacatcattgagaaaggtcctacattcagtgttatatcattcgattcaggcagtgaagaatttgaagaattcatagcaccagatgccatttgcagtttatggaggttatgcatccacgtttacaaggagcaaatttgcttgctttttggatattatagttgtgaggaggagggcatggaaaacattgacttatgggttctgcaagaaaaacggtggaaacaattgtgtccttttatttatgatcccttggatgatttctatcagataatcgggattagtactgacaataaactcataatggcaagaaaagatttcagtaagggcggagtagaactgcaattggggaattacgaatccaaacaagttcttgaaacagggattaagttggccatcatgagatatggcgaaagcgaattcttgtttgcaatgacttacgtagaaagtttagctttactcaataactag

SEQ ID NO: 187 - DNA, 676 bp, Prunus dulcis
  source 1..676: mol_type = genomic DNA
atggcgacgttcattcgctttccttgttctagcttttgctttcttcttgtgtttcattatgagcactggatcttacgtgtattttcaatttgtgcaacaatggccaccgacgacctgcagacttagcagcaaaccttccaaccaacaccggccattacaaagattcaccatccatggcctatggccaagtaactattcaaacccaaggaagcccagtaattgcaatgggtcacaatttaactttatgaaagtgtatcctcagttgcgaaccaaactgaagagatcttggccggacgtggaaggtggcaatgatacaaagttttgggaaggcgaatggaacaagcatggcacatgttccgaacggacgcttaaccaaatgcaatacttcgaggtatcccacgccatgtggaggtcgtacaatattacaaatatccttaaagacgctcatatcgtaccaaatccaacacaaagatggaagtactcggacatagtatcacccattaaaacagcaactggaagaacacccacccttcgttgcaaaactgatccagcaatgcctaacaactctcagttgttacatgaagtggtattttgttatggatataatgcaaaactgcatattgactgtaatcgaacagcaggatgccgaaatcatattgacatcttgtttcaataa

SEQ ID NO: 188 - DNA, 1117 bp, Prunus dulcis
  source 1..1117: mol_type = genomic DNA
atgacattcaaattaggtaagaaagaaatcttgatcgacatcctagcaagactaactgcaaaatccctggttcggtttctttgtacatgcaagtcatggagtgatttgattggcaggtccagttttgtcagcacacacattaataggaatgtcactaaacatgcccacgtctatctactttgcctccaccacccaacttttgaacgtcaaaatgacaacgatgacccatttgttgaagaagaacttctatggtcacttttttcaaatgaaacatttgagcagttctctaagttaagcaatcccttaggaagcacagaacattatgggatatatggttcaagcaatggtctagtttgcatttcggacgagatattgaattatgatagtcctatacacatatggaacccatcggttaggaaatttaggacccctccaataagccccaacaacattaaatttgtctacgttgctctccaattcggcttccactcgggggttaatgactacaaggctgtaagaatgatgcgttccaacaaagataccttcgcggttgaggtttatagtcttagaacagactcttggaagatgattgaaacaattcctccttggttaaaatgcacttggcagcatcatacgggtacattttttaatggagcggcataccgagaaaggtcccttattcagcattatgtccttcgattcaggcagtgaagagttcgaagaattcattgcaccagatgccatttgcaattcacgggggttgtgtatcgacgcttacaaggaccaaatttgcttgcttttcagattttatggttgtgaggaggagggcatgcgaaaagttgacttgtgggttctgcaagaaaaacgatggaaacaattgtgtccttttattattccttggaaatattgccgtacaattgggactagtatagataatgagctcttattgcaaaaaaaagatttcaataagggcgcagcagatctatgtttgtgtaattacgaatccaagcaagttcttgaaacaggaattaagttggccatcatgacatatggcgaaatcgatttcttgttttcatttacttacatagaaagtttggttttactcaataattattaa

SEQ ID NO: 189 - DNA, 743 bp, Prunus dulcis
  source 1..743: mol_type = genomic DNA
agaaagcctctatcattctgttgtatttcttcttgctttagtatttcctaagtatgaaatcgtcactcgctttccttgttcttggttttgctttcttcttttgttacgttatgagcagtggatcttatgactattttcaatttgtgcaacaatggccaccgaccaactgcagagttcgcatgaagcgaccttgctccaatccccggccattacaatatttcacaatccatggcctatggccaagtaatttttcaaacccaacgaagcccagtaattgcaatgggactaaatttgatgcaaggaaagtgtatcctgaaatgcgatccgatctgaagatatcttggccagacgtggaaagtggcaatgatacaaaattttgggaagacgaatggaacaaacacggtacatgttctgaacagacacttaatcaattccagtactttgagcgatcccacgaaatgtggatgtcgtacaatattacagagatccttaaaaacgcttcaatcgtaccacatccggcaaaaacatggacctattcggacatagtatcacccatcaaagcagcaactggaagaacaccgcttcttcgttgcaaatacgataacaacactcagttgttacatgaagtggtattttgttatggctataaagcaataaagcagattgactgtaatcggccaggatgcaaaaataaaattgacatcaagtttcaataaaataatagctagctttctaaaatcgta

SEQ ID NO: 190 - DNA, 1126 bp, Prunus dulcis
  source 1..1126: mol_type = genomic DNA
atgacattcgcactacgtaagaaagaaatcttaatcgacatcctagtaagactacctgcaaaatcccttgttcgatttctgtgtacatgcaagtcatggagtgatttgattggcagctcgagttttgttggcacacaccttcataggaatgtcacgggacatgcccaggcctatctcctttgcctccaccacccaaattttgaatgtcaaagggatgatgatgacccatattttaaagaagaactccaatggtcactgttttccaatgttacatttgaagagagctccaagttaagccatcccttagggagcacagaacattatgtgatatatggttcaagcaatggtttagtttgcatatcggatgagataatgaatttcgatagtcctatacacatatggaacccatcggttaagaaacttagaaccactccaatcagcaccagcattaacattaaatttagccatgttgctctccaattcggtttccactccggggttaatgactactgtaaggatgttgcgtacaaaccaaaatgccttggcggttgagatttatagtctcagaacagactcttggacgatgattgaagcaattcctccttggttaaaatgcacttggcagcatcatcagggtacattttttaatggagtagcataccacatcattgagaaaggtcctacattcagtgttatatcattcgattcaggcagtgaagaatttgaagaattcatagcaccagatgccatttgcagtttatggaggttatgcatccacgtttacaaggagcaaatttgcttgctttttggatattatagttgtgaggaggagggcatggaaaacattgacttatgggttctgcaagaaaaacggtggaaacaattgtgtccttttatttatgatcccttggatgatttctatcagataatcgggattagtactgacaataaactcataatggcaagaaaagatttcagtaagggcggagtagaactgcaattggggaattacgaatccaaacaagttcttgaaacagggattaagttggccatcatgagatatggcgaaagcgaattcttgtttgcaatgacttacgtagaaagtttagctttactcaataactag

SEQ ID NO: 191 - DNA, 1117 bp, Prunus dulcis; identical to SEQ ID NO: 188
  source 1..1117: mol_type = genomic DNA

SEQ ID NO: 192 - DNA, 743 bp, Prunus dulcis; identical to SEQ ID NO: 189
  source 1..743: mol_type = genomic DNA

SEQ ID NO: 193 - DNA, 1126 bp, Prunus dulcis; identical to SEQ ID NO: 190
  source 1..1126: mol_type = genomic DNA

SEQ ID NO: 194 - DNA, 1120 bp, Prunus dulcis
  source 1..1120: mol_type = genomic DNA
atgacattcaaattaggtaagaaagaaatcttgatcgacatcctagcaagactaactgcaaaatccctggttcggtttctttgtacatgcaagtcatggagtgatttgattggcaggtccagttttgtcagcacacacattaataggaatgtcactaaacatgcccacgtctatctactttgcctccaccacccaacttttgaacgtcaaaatgacaacgatgacccatttgttgaagaagaacttctatggtcacttttttcaaatgaaacatttgagcagttctctaagttaagcaatcccttaggaagcacagaacattatgggatatatggttcaagcaatggtctagtttgcatttcggacgagatattgaattatgatagtcctatacacatatggaacccatcggttaggaaatttaggacccctccaataagccccaacaacattaaatttgtctacgttgctctccaattcggcttccactcgggggttaatgactacaaggctgtaagaatgatgcgttccaacaaagataccttcgcggttgaggtttatagtcttagaacagactcttggaagatgattgaaacaattcctccttggttaaaatgcacttggcagcatcatacgggtacattttttaatggagcggcataccactgagaaaggtcccttattcagcattatgtccttcgattcaggcagtgaagagttcgaagaattcattgcaccagatgccatttgcaattcacgggggttgtgtatcgacgcttacaaggaccaaatttgcttgcttttcagattttatggttgtgaggaggagggcatgcgaaaagttgacttgtgggttctgcaagaaaaacgatggaaacaattgtgtccttttattattccttggaaatattgccgtacaattgggactagtatagataatgagctcttattgcaaaaaaaagatttcaataagggcgcagcagatctatgtttgtgtaattacgaatccaagcaagttcttgaaacaggaattaagttggccatcatgacatatggcgaaatcgatttcttgttttcatttacttacatagaaagtttggttttactcaataattattaa

SEQ ID NO: 195 - DNA, 743 bp, Prunus dulcis; identical to SEQ ID NO: 189
  source 1..743: mol_type = genomic DNA

SEQ ID NO: 196 - DNA, 1106 bp, Prunus dulcis
  source 1..1106: mol_type = genomic DNA
atgacattcgcactacgtaagaaagaaatcttaatcgacatcctagtaagactacctgcaaaatcccttgttcgatttctgtgtacatgcaagtcatggagtgatttgattggcagctcgagttttgttggcacacaccttcataggaatgtcacgggacatgcccaggcctatctcctttgcctccaccacccaaattttgaatgtcaaagggatgatgatgacccatattttaaagaagaactccaatggtcactgttttccaatgttacatttgaagagagctccaagttaagccatcccttagggagcacagaacattatgtgatatatggttcaagcaatggtttagtttgcatatcggatgagataatgaatttcgatagtcctatacacatatggaacccatcggttaagaaacttagaaccactccaatcagcaccagcattaacattaaatttagccatgttgctctccaattcggtttccactccggggtttgcgtacaaaccaaaatgccttggcggttgagatttatagtctcagaacagactcttggacgatgattgaagcaattcctccttggttaaaatgcacttggcagcatcatcagggtacattttttaatggagtagcataccacatcattgagaaaggtcctacattcagtgttatatcattcgattcaggcagtgaagaatttgaagaattcatagcaccagatgccatttgcagtttatggaggttatgcatccacgtttacaaggagcaaatttgcttgctttttggatattatagttgtgaggaggagggcatggaaaacattgacttatgggttctgcaagaaaaacggtggaaacaattgtgtccttttatttatgatcccttggatgatttctatcagataatcgggattagtactgacaataaactcataatggcaagaaaagatttcagtaagggcggagtagaactgcaattggggaattacgaatccaaacaagttcttgaaacagggattaagttggccatcatgagatatggcgaaagcgaattcttgtttgcaatgacttacgtagaaagtttagctttactcaataactag

SEQ ID NO: 197 - DNA, 678 bp, Prunus dulcis
  source 1..678: mol_type = genomic DNA
atggcgacgttgtcattcgctttccttgttctagcttttgctttcttcttgtgtttcattatgagcactggatcttacgtgtattttcaatttgtgcaacaatggccaccgacgacctgcagacttagcagcaaaccttccaaccaacaccggccattacaaagattcaccatccatggcctatggccaagtaactattcaaacccaaggaagcccagtaattgcaatgggtcacaatttaactttatgaaagtgtatcctcagttgcgaaccaaactgaagagatcttggccggacgtggaaggtggcaatgatacaaagttttgggaaggcgaatggaacaagcatggcacatgttccgaacggacgcttaaccaaatgcaatacttcgaggtatcccacgccatgtggaggtcgtacaatattacaaatatccttaaagacgctcatatcgtaccaaatccaacacaaagatggaagtactcggacatagtatcacccattaaaacagcaactggaagaacacccacccttcgttgcaaaactgatccagcaatgcctaacaactctcagttgttacatgaagtggtattttgttatggatataatgcaaaactgcatattgactgtaatcgaacagcaggatgccgaaatcatattgacatcttgtttcaataa

(19) United States
(12) Patent Application Publication
(10) Pub. No.: US 2026/0357286 A1
(43) Pub. Date: Nov. 12, 2026

(54) ALMOND BREEDING AND PRODUCTION

(71) Applicant: Ohalo Genetics, Inc., Aptos, CA (US)

(72) Inventors: Franca G. Rossi, Aptos, CA (US); Judson A. Ward, Aptos, CA (US); Lien D. Bertier, Aptos, CA (US)

(73) Assignee: Ohalo Genetics, Inc., Aptos, CA (US)

(21) Appl. No.: 19/691,113

(22) Filed: May 28, 2026

Related U.S. Application Data
(63) Continuation of application No. PCT/US25/37132, filed on Jul. 10, 2025.
(60) Provisional application No. 63/670,291, filed on Jul. 12, 2024.
(60) Provisional application No. 63/784,462, filed on Apr. 7, 2025.

(51) Int. Cl. C12N 15/82; A01H 4/00; C12N 9/22; C12N 15/11

(52) U.S. Cl. CPC C12N 15/8287; A01H 4/008; C12N 9/222; C12N 15/111; C12N 15/8201; C12N 2310/20

(57) ABSTRACT

The present disclosure provides systems and methods for the production of self-fertile almond cultivars (Prunus dulcis) with one or more S-locus haplotypes, wherein one or more genes within the one or more S-locus haplotypes have a deletion as compared to the respective sequence of the unedited gene or genes.

### CROSS REFERENCE TO RELATED MATTER

The present application claims priority to PCT Application No. PCT/US2025/037132, filed Jul. 10, 2025, which claims priority U.S. Application No. 63/670,291, as filed on Jul. 12, 2024, and U.S. Application No. 63/784,462, as filed on Apr. 7, 2025, the entire contents of both applications are incorporated herein by reference for all purposes.

All references, articles, publications, patents, patent publications, and patent applications cited herein within the above text and/or cited below are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### FIELD

The present disclosure relates generally to the field of agricultural science, and more specifically to crop improvement and systems of breeding novel almond cultivars. The present disclosure further relates to plants and plant materials obtained by these processes.

### SUBMISSION OF SEQUENCE LISTING

The Sequence Listing associated with this application is filed in xml electronic format (OHLO.01WOUSU1 Sequence Listing, file size 218000 bytes, created on Apr. 29, 2026) and is hereby incorporated by reference into the specification in its entirety.

### BRIEF DESCRIPTION OF THE FIGURES

The present application can be understood by reference to the following description taken in conjunction with the accompanying FIGURES.

The accompanying FIGURE, which is incorporated herein and form a part of the specification, illustrate some, but not the only or exclusive, example embodiments and/or features. It is intended that the embodiments and FIGURES disclosed herein are to be considered illustrative rather than limiting.

FIG. 1 depicts the % editing efficiency of guided nucleases targeting the S8-RNase gene.

FIG. 2 depicts the % editing efficiency of guided nucleases targeting the S8-SFB gene.

FIG. 3 depicts the % editing efficiency of guided nucleases targeting the S7-RNase gene.

FIG. 4 depicts the % editing efficiency of guided nucleases targeting the S7-SFB gene.

FIG. 5 provides Table 1, which provides a listing of DNA and RNA sequences of the present disclosure.

FIG. 6 depicts Plant 001 with two targeted deletions. One of the deletions is in S7-RNase and disrupts the start codon site. The other deletion is in S8-RNase.

FIG. 7 depicts Plant 002 with one targeted deletion in S8-SFB.

FIG. 8 depicts Plant 003 with three targeted deletions. Two of the deletions is in S7-RNase and disrupts the start codon site. The other deletion is in S7-SFB.

FIG. 9 depicts Plant 004 with two targeted deletions. One of the deletions is in S7-SFB. The other deletion is an S8-SFB.

FIG. 10 depicts Plant 011 with one targeted deletion in S7-RNase and disrupts the start codon site.

FIG. 11 provides Table 2, which provides an alignment of S8-RNase sequences.

FIG. 12 provides Table 3, which provides an alignment of S8-SFB sequences.

FIG. 13 provides Table 4, which provides an alignment of S7-RNAse sequences.

FIG. 14 provides Table 5, which provides an alignment of S7-SFB sequences.

FIG. 15 provides the constructs used in the Split YFP experiments of Example 3.

FIG. 16 provides the Split YFP fluorescence signals resulting from the experiments of Example 3.

FIG. 17 provides the signal peptide prediction for S7-RNase using Signal P (6.0).

FIG. 18 provides an alignment of nucleotide and protein sequences of S7-RNase with the deletion profiles of several edited almond plants using Geneious Prime® 2022.2.1 software.

FIG. 19 provides florescence images of SRNase7_fullYFP (left image, A) and S7-RNAse-SP_fullYFP (right image, B) transiently expressed in almond cells.

### BRIEF DESCRIPTION OF THE SEQUENCE LISTINGS

SEQ ID NO: 1 discloses the transcribed portion (not including the 5′UTR and 3′UTR) of the S8-RNase gene (exons and introns).
SEQ ID NO: 2 discloses the S8-RNase protein coding sequence (exons only).
SEQ ID NO: 3 discloses the S8-RNase amino acid sequence.
SEQ ID NO: 4 discloses the transcribed portion (not including the 5′UTR and 3′UTR) of the S8-SFB gene (no introns, so this also represents the protein coding sequence).
SEQ ID NO: 5 discloses the S8-SFB amino acid sequence.
SEQ ID NO: 6 discloses the transcribed portion (not including the 5′UTR and 3′UTR) of the S7-RNase gene (exons and introns).
SEQ ID NO: 7 discloses the S7-RNase protein coding sequence (exons only).
SEQ ID NO: 8 discloses the S7-RNase amino acid sequence.
SEQ ID NO: 9 discloses the transcribed portion (not including the 5′UTR and 3′UTR) of the S7-SFB gene (no introns, so this also represents the protein coding sequence).
SEQ ID NO: 10 discloses the S7-SFB amino acid sequence.
SEQ ID NO: 11 discloses the S8-RNase guide RNA #1.
SEQ ID NO: 12 discloses the S8-RNase guide RNA #2.
SEQ ID NO: 13 discloses the S8-RNase guide RNA #3.
SEQ ID NO: 14 discloses the S8-RNase guide RNA #4.
SEQ ID NO: 15 discloses the S8-RNase guide RNA #5.
SEQ ID NO: 16 discloses the S8-RNase guide RNA #6.
SEQ ID NO: 17 discloses the S8-RNase guide RNA #7.
SEQ ID NO: 18 discloses the S8-RNase guide RNA #8.
SEQ ID NO: 19 discloses the S8-RNase guide RNA #9.
SEQ ID NO: 20 discloses the S8-RNase guide RNA #10.
SEQ ID NO: 21 discloses the S8-RNase guide RNA #11.
SEQ ID NO: 22 discloses the S8-RNase guide RNA #12.
SEQ ID NO: 23 discloses the S8-RNase guide RNA #13.
SEQ ID NO: 24 discloses the S8-RNase guide RNA #14.
SEQ ID NO: 25 discloses the S8-RNase guide RNA #15.
SEQ ID NO: 26 discloses the S8-RNase guide RNA #16.
SEQ ID NO: 27 discloses the S8-RNase guide RNA #17.
SEQ ID NO: 28 discloses the S8-RNase guide RNA #18.
SEQ ID NO: 29 discloses the S8-RNase guide RNA #19.
SEQ ID NO: 30 discloses the S8-RNase guide RNA #20.
SEQ ID NO: 31 discloses the target site for S8-RNase guide RNA #1.
SEQ ID NO: 32 discloses the target site for S8-RNase guide RNA #2.
SEQ ID NO: 33 discloses the target site for S8-RNase guide RNA #3.
SEQ ID NO: 34 discloses the target site for S8-RNase guide RNA #4.
SEQ ID NO: 35 discloses the target site for S8-RNase guide RNA #5.
SEQ ID NO: 36 discloses the target site for S8-RNase guide RNA #6.
SEQ ID NO: 37 discloses the target site for S8-RNase guide RNA #7.
SEQ ID NO: 38 discloses the target site for S8-RNase guide RNA #8.
SEQ ID NO: 39 discloses the target site for S8-RNase guide RNA #9.
SEQ ID NO: 40 discloses the target site for S8-RNase guide RNA #10.
SEQ ID NO: 41 discloses the target site for S8-RNase guide RNA #11.
SEQ ID NO: 42 discloses the target site for S8-RNase guide RNA #12.
SEQ ID NO: 43 discloses the target site for S8-RNase guide RNA #13.
SEQ ID NO: 44 discloses the target site for S8-RNase guide RNA #14.
SEQ ID NO: 45 discloses the target site for S8-RNase guide RNA #15.
SEQ ID NO: 46 discloses the target site for S8-RNase guide RNA #16.
SEQ ID NO: 47 discloses the target site for S8-RNase guide RNA #17.
SEQ ID NO: 48 discloses the target site for S8-RNase guide RNA #18.
SEQ ID NO: 49 discloses the target site for S8-RNase guide RNA #19.
SEQ ID NO: 50 discloses the target site for S8-RNase guide RNA #20.
SEQ ID NO: 51 discloses the S8-SFB guide RNA #1.
SEQ ID NO: 52 discloses the S8-SFB guide RNA #2.
SEQ ID NO: 53 discloses the S8-SFB guide RNA #3.
SEQ ID NO: 54 discloses the S8-SFB guide RNA #4.
SEQ ID NO: 55 discloses the S8-SFB guide RNA #5.
SEQ ID NO: 56 discloses the S8-SFB guide RNA #6.
SEQ ID NO: 57 discloses the S8-SFB guide RNA #7.
SEQ ID NO: 58 discloses the S8-SFB guide RNA #8.
SEQ ID NO: 59 discloses the S8-SFB guide RNA #9.
SEQ ID NO: 60 discloses the S8-SFB guide RNA #10.
SEQ ID NO: 61 discloses the S8-SFB guide RNA #11.
SEQ ID NO: 62 discloses the S8-SFB guide RNA #12.
SEQ ID NO: 63 discloses the S8-SFB guide RNA #13.
SEQ ID NO: 64 discloses the target site for S8-SFB guide RNA #1.
SEQ ID NO: 65 discloses the target site for S8-SFB guide RNA #2.
SEQ ID NO: 66 discloses the target site for S8-SFB guide RNA #3.
SEQ ID NO: 67 discloses the target site for S8-SFB guide RNA #4.
SEQ ID NO: 68 discloses the target site for S8-SFB guide RNA #5.
SEQ ID NO: 69 discloses the target site for S8-SFB guide RNA #6.
SEQ ID NO: 70 discloses the target site for S8-SFB guide RNA #7.
SEQ ID NO: 71 discloses the target site for S8-SFB guide RNA #8.
SEQ ID NO: 72 discloses the target site for S8-SFB guide RNA #9.
SEQ ID NO: 73 discloses the target site for S8-SFB guide RNA #10.
SEQ ID NO: 74 discloses the target site for S8-SFB guide RNA #11.
SEQ ID NO: 75 discloses the target site for S8-SFB guide RNA #12.
SEQ ID NO: 76 discloses the target site for S8-SFB guide RNA #13.
SEQ ID NO: 77 discloses the S7-RNase guide RNA #1.
SEQ ID NO: 78 discloses the S7-RNase guide RNA #2.
SEQ ID NO: 79 discloses the S7-SFB guide RNA #17.
SEQ ID NO: 80 discloses the S7-RNase guide RNA #4.
SEQ ID NO: 81 discloses the S7-RNase guide RNA #5.
SEQ ID NO: 82 discloses the S7-RNase guide RNA #6.
SEQ ID NO: 83 discloses the S7-RNase guide RNA #7.
SEQ ID NO: 84 discloses the S7-RNase guide RNA #8.
SEQ ID NO: 85 discloses the S7-RNase guide RNA #9.
SEQ ID NO: 86 discloses the S7-RNase guide RNA #10.
SEQ ID NO: 87 discloses the S7-RNase guide RNA #11.
SEQ ID NO: 88 discloses the S7-RNase guide RNA #12.
SEQ ID NO: 89 discloses the S7-RNase guide RNA #13.
SEQ ID NO: 90 discloses the S7-RNase guide RNA #14.
SEQ ID NO: 91 discloses the S7-RNase guide RNA #15.
SEQ ID NO: 92 discloses the S7-RNase guide RNA #16.
SEQ ID NO: 93 discloses the S7-RNase guide RNA #17.
SEQ ID NO: 94 discloses the S7-RNase guide RNA #18.
SEQ ID NO: 95 discloses the S7-RNase guide RNA #19.
SEQ ID NO: 96 discloses the S7-RNase guide RNA #20.
SEQ ID NO: 97 discloses the S7-RNase guide RNA #21.
SEQ ID NO: 98 discloses the target site for S7-RNase guide RNA #1.
SEQ ID NO: 99 discloses the target site for S7-RNase guide RNA #2.
SEQ ID NO: 100 discloses the target site for S7-SFB guide RNA #17.
SEQ ID NO: 101 discloses the target site for S7-RNase guide RNA #4.
SEQ ID NO: 102 discloses the target site for S7-RNase guide RNA #5.
SEQ ID NO: 103 discloses the target site for S7-RNase guide RNA #6.
SEQ ID NO: 104 discloses the target site for S7-RNase guide RNA #7.
SEQ ID NO: 105 discloses the target site for S7-RNase guide RNA #8.
SEQ ID NO: 106 discloses the target site for S7-RNase guide RNA #9.
SEQ ID NO: 107 discloses the target site for S7-RNase guide RNA #10.
SEQ ID NO: 108 discloses the target site for S7-RNase guide RNA #11.
SEQ ID NO: 109 discloses the target site for S7-RNase guide RNA #12.
SEQ ID NO: 110 discloses the target site for S7-RNase guide RNA #13.
SEQ ID NO: 111 discloses the target site for S7-RNase guide RNA #14.
SEQ ID NO: 112 discloses the target site for S7-RNase guide RNA #15.
SEQ ID NO: 113 discloses the target site for S7-RNase guide RNA #16.
SEQ ID NO: 114 discloses the target site for S7-RNase guide RNA #17.
SEQ ID NO: 115 discloses the target site for S7-RNase guide RNA #18.
SEQ ID NO: 116 discloses the target site for S7-RNase guide RNA #19.
SEQ ID NO: 117 discloses the target site for S7-RNase guide RNA #20.
SEQ ID NO: 118 discloses the target site for S7-RNase guide RNA #21.
SEQ ID NO: 119 discloses the S7-SFB guide RNA #1.
SEQ ID NO: 120 discloses the S7-SFB guide RNA #2.
SEQ ID NO: 121 discloses the S7-SFB guide RNA #3.
SEQ ID NO: 122 discloses the S7-SFB guide RNA #4.
SEQ ID NO: 123 discloses the S7-SFB guide RNA #5.
SEQ ID NO: 124 discloses the S7-SFB guide RNA #6.
SEQ ID NO: 125 discloses the S7-SFB guide RNA #7.
SEQ ID NO: 126 discloses the S7-SFB guide RNA #8.
SEQ ID NO: 127 discloses the S7-SFB guide RNA #9.
SEQ ID NO: 128 discloses the S7-SFB guide RNA #10.
SEQ ID NO: 129 discloses the S7-SFB guide RNA #11.
SEQ ID NO: 130 discloses the S7-SFB guide RNA #12.
SEQ ID NO: 131 discloses the S7-SFB guide RNA #13.
SEQ ID NO: 132 discloses the S7-SFB guide RNA #14.
SEQ ID NO: 133 discloses the S7-SFB guide RNA #15.
SEQ ID NO: 134 discloses the S7-SFB guide RNA #16.
SEQ ID NO: 135 discloses the target site for S7-SFB guide RNA #1.
SEQ ID NO: 136 discloses the target site for S7-SFB guide RNA #2.
SEQ ID NO: 137 discloses the target site for S7-SFB guide RNA #3.
SEQ ID NO: 138 discloses the target site for S7-SFB guide RNA #4.
SEQ ID NO: 139 discloses the target site for S7-SFB guide RNA #5.
SEQ ID NO: 140 discloses the target site for S7-SFB guide RNA #6.
SEQ ID NO: 141 discloses the target site for S7-SFB guide RNA #7.
SEQ ID NO: 142 discloses the target site for S7-SFB guide RNA #8.
SEQ ID NO: 143 discloses the target site for S7-SFB guide RNA #9.
SEQ ID NO: 144 discloses the target site for S7-SFB guide RNA #10.
SEQ ID NO: 145 discloses the target site for S7-SFB guide RNA #11.
SEQ ID NO: 146 discloses the target site for S7-SFB guide RNA #12.
SEQ ID NO: 147 discloses the target site for S7-SFB guide RNA #13.
SEQ ID NO: 148 discloses the target site for S7-SFB guide RNA #14.
SEQ ID NO: 149 discloses the target site for S7-SFB guide RNA #15.
SEQ ID NO: 150 discloses the target site for S7-SFB guide RNA #16.
SEQ ID NO: 151 discloses the sequence of the region immediately upstream of start codon for S8-RNase.
SEQ ID NO: 152 discloses the sequence of the region immediately upstream of start codon for S7-RNase.
SEQ ID NO: 153 discloses the alignment sequence for the S7-SFB polynucleotide of Nonpareil (ref).
SEQ ID NO: 154 discloses the alignment sequence for the S7-RNase polynucleotide of Nonpareil (ref).
SEQ ID NO: 155 discloses the alignment sequence for the S8-SFB polynucleotide of Nonpareil (ref).
SEQ ID NO: 156 discloses the alignment sequence for the S8-RNase polynucleotide of Nonpareil (ref).
SEQ ID NO: 157 discloses the alignment sequence for the S7-SFB polypeptide of Nonpareil (ref).
SEQ ID NO: 158 discloses the alignment sequence for the S7-RNase polypeptide of Nonpareil (ref).
SEQ ID NO: 159 discloses the alignment sequence for the S8-SFB polypeptide of Nonpareil (ref).
SEQ ID NO: 160 discloses the alignment sequence for the S8-RNase polypeptide of Nonpareil (ref).
SEQ ID NO: 161 discloses the alignment sequence for the S8-SFB polynucleotide of Plant 002.
SEQ ID NO: 162 discloses the alignment sequence for the S7-RNase polynucleotide of Plant 001.
SEQ ID NO: 163 discloses the alignment sequence for the S8-RNase polynucleotide of Plant 001.
SEQ ID NO: 164 discloses the alignment sequence for the S8-SFB polynucleotide of Plant 006.
SEQ ID NO: 165 discloses the alignment sequence for the S8-RNase polynucleotide of Plant 006.
SEQ ID NO: 166 discloses the alignment sequence for the S7-SFB polynucleotide of Plant 007.
SEQ ID NO: 167 discloses the alignment sequence for the S8-SFB polynucleotide of Plant 008.
SEQ ID NO: 168 discloses the alignment sequence for the S8-RNase polynucleotide of Plant 008.
SEQ ID NO: 169 discloses the alignment sequence for the S7-SFB polynucleotide of Plant 009.
SEQ ID NO: 170 discloses the alignment sequence for the S7-RNase polynucleotide of Plant 009.
SEQ ID NO: 171 discloses the alignment sequence for the S8-SFB polynucleotide of Plant 009.
SEQ ID NO: 172 discloses the alignment sequence for the S8-RNase polynucleotide of Plant 009.
SEQ ID NO: 173 discloses the alignment sequence for the S7-SFB polynucleotide of Plant 004.
SEQ ID NO: 174 discloses the alignment sequence for the S8-SFB polynucleotide of Plant 004.
SEQ ID NO: 175 discloses the alignment sequence for the S8-SFB polynucleotide of Plant 010.
SEQ ID NO: 176 discloses the alignment sequence for the S7-SFB polynucleotide of Plant 003.
SEQ ID NO: 177 discloses the alignment sequence for the S7-RNase polynucleotide of Plant 003.
SEQ ID NO: 178 discloses the alignment sequence for the S7-RNase polynucleotide of Plant 011.
SEQ ID NO: 179 discloses the alignment sequence for the S7-RNase polynucleotide of Plant 011.
SEQ ID NO: 180 discloses the alignment sequence for the S7-SFB polynucleotide of Plant 012.
SEQ ID NO: 181 discloses the alignment sequence for the S7-RNase polynucleotide of Plant 012.
SEQ ID NO: 182 discloses the alignment sequence for the S8-RNase polynucleotide of Plant 012.
SEQ ID NO: 183 discloses the alignment sequence for the S7-RNase polynucleotide of Plant 013.
SEQ ID NO: 184 discloses the alignment sequence for the S8-RNase polynucleotide of Plant 013.
SEQ ID NO: 185 discloses the alignment sequence for the S7-SFB polynucleotide of Plant 014.
SEQ ID NO: 186 discloses the alignment sequence for the S8-SFB polynucleotide of Plant 014.
SEQ ID NO: 187 discloses the alignment sequence for the S8-RNase polynucleotide of Plant 014.
SEQ ID NO: 188 discloses the alignment sequence for the S7-SFB polynucleotide of Plant 015.
SEQ ID NO: 189 discloses the alignment sequence for the S7-RNase polynucleotide of Plant 015.
SEQ ID NO: 190 discloses the alignment sequence for the S8-SFB polynucleotide of Plant 015.
SEQ ID NO: 191 discloses the alignment sequence for the S7-SFB polynucleotide of Plant 016.
SEQ ID NO: 192 discloses the alignment sequence for the S7-RNase polynucleotide of Plant 016.
SEQ ID NO: 193 discloses the alignment sequence for the S8-SFB polynucleotide of Plant 016.
SEQ ID NO: 194 discloses the alignment sequence for the S7-SFB polynucleotide of Plant 017.
SEQ ID NO: 195 discloses the alignment sequence for the S7-RNase polynucleotide of Plant 017.
SEQ ID NO: 196 discloses the alignment sequence for the S8-SFB polynucleotide of Plant 017.
SEQ ID NO: 197 discloses the alignment sequence for the S8-RNase polynucleotide of Plant 017.

### BRIEF SUMMARY

Provided herein are compositions, methods, kits, and genomes directed to the generation of self-fertile almond plants.

The following embodiments and aspects thereof are described and illustrated in conjunction with systems, tools and methods, which are meant to be exemplary and illustrative, not limiting in scope.

In some aspects of the present disclosure is provided, a self-fertile almond plant comprising one or more S-locus haplotypes wherein the one or more S-locus haplotypes comprise a deletion of one or more genes.

In some aspects of the present disclosure is provided, a self-fertile almond plant comprising one or more S-locus haplotypes, wherein the one or more S-locus haplotypes comprise a deletion of one or more genes, and wherein said deletion results in a frameshift mutation.

In some aspects of the present disclosure is provided, a self-fertile almond plant comprising one or more S-locus haplotypes, wherein the one or more S-locus haplotypes comprise a deletion of one or more genes, and wherein said deletion results in an in-frame mutation.

In some aspects of the present disclosure is provided, a self-fertile almond plant comprising one or more S-locus haplotypes, wherein the one or more S-locus haplotypes comprise a deletion of one or more genes, wherein said deletion results in a frameshift mutation, and wherein said deletion is 1 bp, 2 bp, 4 bp, 5 bp, 7 bp, 8 bp, 10 bp, 11 bp, 13 bp, 14 bp, 16 bp, 17 bp, 19 bp, 20 bp, 22 bp, 23 bp, 25 bp, 26 bp, 28 bp, 29, 31 bp, 32 bp, 34 bp or 35 bp in length.

In some aspects of the present disclosure is provided, a self-fertile almond plant comprising one or more S-locus haplotypes, wherein the one or more S-locus haplotypes comprise a deletion of an S8-RNase gene with a transcribed portion, wherein the transcribed portion comprises a DNA sequence with at least 90%, at least 95%, at least 99% or 100% sequence identity to SEQ ID NO: 1

In some aspects of the present disclosure is provided, a self-fertile almond plant comprising one or more S-locus haplotypes, wherein the one or more S-locus haplotypes comprise a deletion of an S8-RNase gene comprising a DNA protein coding sequence with at least 90%, at least 95%, at least 99% or 100% sequence identity to SEQ ID NO: 2.

In some aspects of the present disclosure is provided, a self-fertile almond plant comprising one or more S-locus haplotypes, wherein the one or more S-locus haplotypes comprise a deletion of a gene encoding an S8-RNase amino acid sequence with at least 90%, at least 95%, at least 99% or 100% sequence identity to SEQ ID NO: 3.

In some aspects of the present disclosure is provided, a self-fertile almond plant comprising one or more S-locus haplotypes, wherein the one or more S-locus haplotypes comprise a deletion in an S8-SFB gene with a transcribed portion, wherein the transcribed portion comprises a DNA sequence with at least 90%, at least 95%, at least 99% or 100% sequence identity to SEQ ID NO: 4

In some aspects of the present disclosure is provided, a self-fertile almond plant comprising one or more S-locus haplotypes, wherein the one or more S-locus haplotypes comprise a deletion in a gene encoding an S8-SFB amino acid sequence that has at least 90%, at least 95%, at least 99% or 100% sequence identity to SEQ ID NO: 5.

In some aspects of the present disclosure is provided, a self-fertile almond plant comprising one or more S-locus haplotypes, wherein the one or more S-locus haplotypes comprise a deletion of an S7-RNAse gene with a transcribed portion, wherein the transcribed portion comprises a DNA sequence with at least 90%, at least 95%, at least 99% or 100% sequence identity to SEQ ID NO: 6.

In some aspects of the present disclosure is provided, a self-fertile almond plant comprising one or more S-locus haplotypes, wherein the one or more S-locus haplotypes comprise a deletion of an S7-RNAse gene comprising a DNA protein coding sequence with at least 90%, at least 95%, at least 99% or 100% sequence identity to SEQ ID NO: 7.

In some aspects of the present disclosure is provided, a self-fertile almond plant comprising one or more S-locus haplotypes, wherein the one or more S-locus haplotypes comprise a deletion of a gene encoding an S7-RNAse amino acid sequence with at least 90%, at least 95%, at least 99% or 100% sequence identity to SEQ ID NO: 8.

In some aspects of the present disclosure is provided, a self-fertile almond plant comprising one or more S-locus haplotypes, wherein the one or more S-locus haplotypes comprise a deletion in an S7-SFB gene with a transcribed portion, wherein the transcribed portion comprises a DNA sequence with at least 90%, at least 95%, at least 99% or 100% sequence identity to SEQ ID NO: 9.

In some aspects of the present disclosure is provided, a self-fertile almond plant comprising one or more S-locus haplotypes, wherein the one or more S-locus haplotypes comprise a deletion in a gene encoding an S7-SFB amino acid sequence that has at least 90%, at least 95%, at least 99% or 100% sequence identity to SEQ ID NO: 10.

In some aspects of the present disclosure is provided, a self-fertile almond plant, wherein said two or more S-locus haplotypes comprise: an S8-locus, wherein said S8-locus includes a gene encoding an S-RNase protein and an S7-SFB gene with a transcribed portion, wherein the transcribed portion of the S7-SFB gene comprises a DNA sequence with at least 90%, at least 95%, at least 99% or 100% sequence identity to SEQ ID NO: 1; wherein the S7-SFB gene comprises a DNA protein coding sequence with at least 90%, at least 95%, at least 99% or 100% sequence identity to SEQ ID NO: 2 and wherein the S7-SFB amino acid sequence has at least 90%, at least 95%, at least 99% or 100% sequence identity to SEQ ID NO: 3; and an S7-locus, wherein said S7-locus includes a gene encoding an S-RNase protein and an S7-RNase gene with a transcribed portion, wherein the transcribed portion of the S7-RNase gene comprises a DNA sequence with at least 90%, at least 95%, at least 99% or 100% sequence identity to SEQ ID NO: 6, wherein the S7-RNase gene comprises a DNA protein coding sequence with at least 90%, at least 95%, at least 99% or 100% sequence identity to SEQ ID NO: 7 and the S7-RNase amino acid sequence has at least 90%, at least 95%, at least 99% or 100% sequence identity SEQ ID NO: 8.

In some aspects of the present disclosure is provided, a self-fertile almond plant, wherein said two or more S-locus haplotypes, wherein one of said two or more S-locus haplotypes comprise: an S8-locus comprising a gene encoding an SFB protein and an S8-SFB gene with a transcribed portion, wherein the transcribed portion of the S8-SFB gene comprises a DNA sequence with at least 90%, at least 95%, at least 99% or 100% sequence identity to SEQ ID NO: 4 and said S8-SFB protein has at least 90%, at least 95%, at least 99% or 100% sequence identity to SEQ ID NO: 5; and an S7-locus comprising an S7-SFB gene encoding an SFB protein and an S7-SFB gene with a transcribed portion, wherein the transcribed portion of the S7-SFB gene comprises a DNA sequence with at least 90%, at least 95%, at least 99% or 100% sequence identity to SEQ ID NO: 9 and said S7-SFB protein has at least 90%, at least 95%, at least 99% or 100% sequence identity SEQ ID NO: 10.

In some aspects of the present disclosure is provided, a self-fertile almond plant, wherein the deletion begins in a region between about 20 bp upstream, and about 20 bp downstream from the 3′ end of a target site for a guided nuclease, wherein the guided nuclease comprises a guide nucleic acid, wherein the guide nucleic acid comprises a guide RNA, wherein the guide RNA comprises an RNA sequence with at least 90%, at least 95%, at least 99% or 100% sequence identity to SEQ ID NO: 11, SEQ ID NO: 12, SEQ ID NO: 13, SEQ ID NO: 14, SEQ ID NO: 15 SEQ ID NO: 16, SEQ ID NO: 17, SEQ ID NO: 18, SEQ ID NO: 19, SEQ ID NO: 20, SEQ ID NO: 21 SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96 and SEQ ID NO: 97 and SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133 and SEQ ID NO: 134 and wherein the target site comprises a DNA sequence with at least 90%, at least 95%, at least 99% or 100% sequence identity SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118 and SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, and SEQ ID NO: 150.

In some aspects of the present disclosure is provided, a modified almond plant, plant part, or plant cell, wherein the almond plant, plant part, or plant cell is derived from a ‘Nonpareil’ type breeding line.

In some aspects of the present disclosure is provided, a processed almond product derived from the modified almond plant, plant part, or plant cell, wherein the processed almond product comprises a detectable amount of at least one of the one or more S-locus haplotypes of the modified plant, plant part, or plant cell, wherein the processed almond product is selected from the group consisting of seed, nuts, butters, oils, milk, cosmetics, extract, isolate, food, livestock feed, or a raw material for industry, wherein the processed almond product is non-regenerable.

In some aspects of the present disclosure is provided, a method of producing a modified almond plant, plant part, or plant cell, the method comprising: introducing into an almond cell one or more S-locus haplotypes, wherein the one or more S-locus haplotypes comprise a deletion of one or more genes; and regenerating a modified almond plant, plant, part, or plant cell from the almond cell, wherein the modified almond plant, plant, part, or plant cell comprises one or more S-locus haplotypes, wherein one or more genes within the one or more S-locus haplotypes comprise a deletion as compared to the respective sequence of the one or more parent genes.

In some aspects of the present disclosure is provided, a method of producing a modified almond plant, plant part, or plant cell, the method comprising: introducing into an almond cell one or more S-locus haplotypes, wherein the one or more S-locus haplotypes comprise a deletion of one or more genes and wherein said deletion results in a frameshift mutation; and regenerating a modified almond plant, plant, part, or plant cell from the almond cell, wherein the modified almond plant, plant, part, or plant cell comprises one or more S-locus haplotypes, wherein one or more genes within the one or more S-locus haplotypes comprise a deletion as compared to the respective sequence of the one or more parent genes and wherein said deletion results in a frameshift mutation.

In some aspects of the present disclosure is provided, a method of producing a modified almond plant, plant part, or plant cell, the method comprising: introducing into an almond cell one or more S-locus haplotypes, wherein the one or more S-locus haplotypes comprise a deletion of one or more genes and wherein said deletion results in an in-frame mutation; and regenerating a modified almond plant, plant, part, or plant cell from the almond cell, wherein the modified almond plant, plant, part, or plant cell comprises one or more S-locus haplotypes, wherein one or more genes within the one or more S-locus haplotypes comprise a deletion as compared to the respective sequence of the one or more parent genes and wherein said deletion results in an in-frame mutation.

In some aspects of the present disclosure is provided, a method of producing a modified almond plant, plant part, or plant cell, the method comprising: introducing into an almond cell one or more S-locus haplotypes, wherein the one or more S-locus haplotypes comprise a deletion of one or more genes and wherein said deletion results in a frameshift mutation and wherein said deletion is 1 bp, 2 bp, 4 bp, 5 bp, 7 bp, 8 bp, 10 bp, 11 bp, 13 bp, 14 bp, 16 bp, 17 bp, 19 bp, 20 bp, 22 bp, 23 bp, 25 bp, 26 bp, 28 bp, 29, 31 bp, 32 bp, 34 bp or 35 bp in length; and regenerating a modified almond plant, plant, part, or plant cell from the almond cell, wherein the modified almond plant, plant, part, or plant cell comprises one or more S-locus haplotypes, wherein one or more genes within the one or more S-locus haplotypes comprise a deletion as compared to the respective sequence of the one or more parent genes and wherein said deletion results in a frameshift mutation and wherein said deletion is 1 bp, 2 bp, 4 bp, 5 bp, 7 bp, 8 bp, 10 bp, 11 bp, 13 bp, 14 bp, 16 bp, 17 bp, 19 bp, 20 bp, 22 bp, 23 bp, 25 bp, 26 bp, 28 bp, 29, 31 bp, 32 bp, 34 bp or 35 bp in length.

In some aspects of the present disclosure is provided, a method of producing a modified almond plant, plant part, or plant cell, the method comprising: introducing into an almond cell one or more S-locus haplotypes, wherein the one or more S-locus haplotypes comprise a deletion of one or more genes and wherein said deletion results in an in-frame mutation and wherein said deletion is 1 bp, 2 bp, 4 bp, 5 bp, 7 bp, 8 bp, 10 bp, 11 bp, 13 bp, 14 bp, 16 bp, 17 bp, 19 bp, 20 bp, 22 bp, 23 bp, 25 bp, 26 bp, 28 bp, 29, 31 bp, 32 bp, 34 bp or 35 bp in length; and regenerating a modified almond plant, plant, part, or plant cell from the almond cell, wherein the modified almond plant, plant, part, or plant cell comprises one or more S-locus haplotypes, wherein one or more genes within the one or more S-locus haplotypes comprise a deletion as compared to the respective sequence of the one or more parent genes and wherein said deletion results in an in-frame mutation and wherein said deletion is 1 bp, 2 bp, 4 bp, 5 bp, 7 bp, 8 bp, 10 bp, 11 bp, 13 bp, 14 bp, 16 bp, 17 bp, 19 bp, 20 bp, 22 bp, 23 bp, 25 bp, 26 bp, 28 bp, 29, 31 bp, 32 bp, 34 bp or 35 bp in length.

In some aspects of the present disclosure is provided, an almond genome, characterized by comprising one or more S-locus haplotypes wherein the one or more S-locus haplotypes comprise a deletion of one or more genes.

In some aspects of the present disclosure is provided, an almond genome, characterized by comprising one or more S-locus haplotypes, wherein the one or more S-locus haplotypes comprise a deletion of one or more genes, and wherein said deletion results in a frameshift mutation.

In some aspects of the present disclosure is provided, an almond genome, characterized by comprising one or more S-locus haplotypes, wherein the one or more S-locus haplotypes comprise a deletion of one or more genes, and wherein said deletion results in an in-frame mutation

In some aspects of the present disclosure is provided, an almond genome, characterized by comprising one or more S-locus haplotypes, wherein the one or more S-locus haplotypes comprise a deletion of one or more genes, wherein said deletion results in a frameshift mutation, and wherein said deletion is 1 bp, 2 bp, 4 bp, 5 bp, 7 bp, 8 bp, 10 bp, 11 bp, 13 bp, 14 bp, 16 bp, 17 bp, 19 bp, 20 bp, 22 bp, 23 bp, 25 bp, 26 bp, 28 bp, 29, 31 bp, 32 bp, 34 bp or 35 bp in length.

In some aspects of the present disclosure is provided, an almond genome, characterized by comprising one or more S-locus haplotypes, wherein the one or more S-locus haplotypes comprise a deletion of an S8-RNase gene with a transcribed portion, wherein the transcribed portion comprises a DNA sequence with at least 90%, at least 95%, at least 99% or 100% sequence identity to SEQ ID NO: 1

In some aspects of the present disclosure is provided, an almond genome, characterized by comprising one or more S-locus haplotypes, wherein the one or more S-locus haplotypes comprise a deletion of an S8-RNase gene comprising a DNA protein coding sequence with at least 90%, at least 95%, at least 99% or 100% sequence identity to SEQ ID NO: 2.

In some aspects of the present disclosure is provided, an almond genome, characterized by comprising one or more S-locus haplotypes, wherein the one or more S-locus haplotypes comprise a deletion of a gene encoding an S8-RNase amino acid sequence with at least 90%, at least 95%, at least 99% or 100% sequence identity to SEQ ID NO: 3.

In some aspects of the present disclosure is provided, an almond genome, characterized by comprising one or more S-locus haplotypes, wherein the one or more S-locus haplotypes comprise a deletion in an S8-SFB gene with a transcribed portion, wherein the transcribed portion comprises a DNA sequence with at least 90%, at least 95%, at least 99% or 100% sequence identity to SEQ ID NO: 4

In some aspects of the present disclosure is provided, an almond genome, characterized by comprising one or more S-locus haplotypes, wherein the one or more S-locus haplotypes comprise a deletion in a gene encoding an S8-SFB amino acid sequence that has at least 90%, at least 95%, at least 99% or 100% sequence identity to SEQ ID NO: 5.

In some aspects of the present disclosure is provided, an almond genome, characterized by comprising one or more S-locus haplotypes, wherein the one or more S-locus haplotypes comprise a deletion of an S7-RNAse gene with a transcribed portion, wherein the transcribed portion comprises a DNA sequence with at least 90%, at least 95%, at least 99% or 100% sequence identity to SEQ ID NO: 6

In some aspects of the present disclosure is provided, an almond genome, characterized by comprising one or more S-locus haplotypes, wherein the one or more S-locus haplotypes comprise a deletion of an S7-RNAse gene comprising a DNA protein coding sequence with at least 90%, at least 95%, at least 99% or 100% sequence identity to SEQ ID NO: 7.

In some aspects of the present disclosure is provided, an almond genome, characterized by comprising one or more S-locus haplotypes, wherein the one or more S-locus haplotypes comprise a deletion of a gene encoding an S7-RNAse amino acid sequence with at least 90%, at least 95%, at least 99% or 100% sequence identity to SEQ ID NO: 8.

In some aspects of the present disclosure is provided, an almond genome, characterized by comprising one or more S-locus haplotypes, wherein the one or more S-locus haplotypes comprise a deletion in an S7-SFB gene with a transcribed portion, wherein the transcribed portion comprises a DNA sequence with at least 90%, at least 95%, at least 99% or 100% sequence identity to SEQ ID NO: 9.

In some aspects of the present disclosure is provided, an almond genome, characterized by comprising one or more S-locus haplotypes, wherein the one or more S-locus haplotypes comprise a deletion in a gene encoding an S7-SFB amino acid sequence that has at least 90%, at least 95%, at least 99% or 100% sequence identity to SEQ ID NO: 10.

In some aspects of the present disclosure is provided, an almond genome, wherein said two or more S-locus haplotypes comprise: an S8-locus, wherein said S8-locus includes a gene encoding an S-RNase protein and an S7-SFB gene with a transcribed portion, wherein the transcribed portion of the S7-SFB gene comprises a DNA sequence with at least 90%, at least 95%, at least 99% or 100% sequence identity to SEQ ID NO: 1; wherein the S7-SFB gene comprises a DNA protein coding sequence with at least 90%, at least 95%, at least 99% or 100% sequence identity to SEQ ID NO: 2 and wherein the S7-SFB amino acid sequence has at least 90%, at least 95%, at least 99% or 100% sequence identity to SEQ ID NO: 3; and an S7-locus, wherein said S7-locus includes a gene encoding an S-RNase protein and an S7-RNase gene with a transcribed portion, wherein the transcribed portion of the S7-RNase gene comprises a DNA sequence with at least 90%, at least 95%, at least 99% or 100% sequence identity to SEQ ID NO: 6, wherein the S7-RNase gene comprises a DNA protein coding sequence with at least 90%, at least 95%, at least 99% or 100% sequence identity to SEQ ID NO: 7 and the S7-RNase amino acid sequence has at least 90%, at least 95%, at least 99% or 100% sequence identity SEQ ID NO: 8.

In some aspects of the present disclosure is provided, an almond genome of claim 66, wherein said two or more S-locus haplotypes, wherein one of said two or more S-locus haplotypes comprise: an S8-locus comprising a gene encoding an SFB protein and an S8-SFB gene with a transcribed portion, wherein the transcribed portion of the S8-SFB gene comprises a DNA sequence with at least 90%, at least 95%, at least 99% or 100% sequence identity to SEQ ID NO: 4 and said S8-SFB protein has at least 90%, at least 95%, at least 99% or 100% sequence identity to SEQ ID NO: 5; and an S7-locus comprising an S7-SFB gene encoding an SFB protein and an S7-SFB gene with a transcribed portion, wherein the transcribed portion of the S7-SFB gene comprises a DNA sequence with at least 90%, at least 95%, at least 99% or 100% sequence identity to SEQ ID NO: 9 and said S7-SFB protein has at least 90%, at least 95%, at least 99% or 100% sequence identity SEQ ID NO: 10.

In some aspects of the present disclosure is provided, a guide RNA comprising an RNA sequence with at least 90%, at least 95%, at least 99% or 100% sequence identity to SEQ ID NO: 11, SEQ ID NO: 12, SEQ ID NO: 13, SEQ ID NO: 14, SEQ ID NO: 15 SEQ ID NO: 16, SEQ ID NO: 17, SEQ ID NO: 18, SEQ ID NO: 19, SEQ ID NO: 20, SEQ ID NO: 21 SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96 and SEQ ID NO: 97 and SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133 and SEQ ID NO: 134; recombinant DNA construct comprising a first expression cassette comprising a first DNA sequence encoding the guide RNA; wherein the first DNA sequence is operably linked to a first plant-expressible promoter; a vector comprising the recombinant DNA construct; and a host cell comprising the vector.

In some aspects of the present disclosure is provided, a kit for producing modified almond plant, plant part, or plant cell, the kit comprising one or more of the guide RNAs, a recombinant DNA construct, a vector, and a host cell.

### DEFINITIONS

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used in this specification, the term “and/or” is used in this disclosure to mean either “and” or “or” unless indicated otherwise.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “allele(s)” refers to any of one or more alternative forms of a gene at a particular locus. In a diploid (or amphidiploid) cell of an organism, alleles of a given gene are located at a specific location or locus on a chromosome, with one allele being present on each chromosome of the pair of homologous chromosomes. Similarly, in a tetraploid cell of an organism, one allele is present on each chromosome of the group of four homologous chromosomes. “Heterozygous” alleles are different alleles residing at a specific locus, positioned individually on corresponding homologous chromosomes. “Homozygous” alleles are identical alleles residing at a specific locus, positioned individually on corresponding homologous chromosomes in the cell.

As used herein, “almond” typically refers to the species Prunus dulcis. The terms “almond”, “almond plant” and “almond tree” include the whole almond plant or any parts or derivatives thereof, such as plant organs (e.g., harvested or non-harvested flowers, leaves, etc.), plant cells, plant protoplasts, plant cell or tissue cultures from which whole plants can be regenerated, regenerable or non-regenerable plant cells, plant calli, plant cell clumps, and plant cells that are intact in plants, or parts of plants, such as embryos, pollen, ovules, ovaries (e.g., harvested tissues or organs), flowers, leaves, seeds, clonally propagated plants, roots, stems, cotyledons, hypocotyls, root tips, meristems, nodes, stolon tips and the like. The almond plant parts or derivatives thereof can also include any of the aforementioned plant parts in an encapsulated form such as, for example, shoot meristems, nodes, stolon tips, and the like, encapsulated in alginate, e.g., in a synthetic seed. Any developmental stage is also included, such as seedlings, immature and mature, etc.

As used herein, the term “at least a portion” or “fragment” of a nucleic acid or polypeptide means a portion having the minimal size characteristics of such sequences, or any larger fragment of the full-length molecule, up to and including the full-length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full-length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids. In some embodiments, a fragment of a polypeptide or polynucleotide comprises at least 10%, 20%, 30%, 40%, 50%, 60%, 70%, 80%, 90%, 95%, 96%, 97%, 98%, or 99% of the entire length of the reference polypeptide or polynucleotide. In some embodiments, a polypeptide or polynucleotide fragment may contain 5, 10, 15, 20, 25, 30, 35, 40, 45, 50, 60, 70, 80, 90, 100, 200, 300, 400, 500, 600, 700, 800, 900, 1000, 2000 or more nucleotides or amino acids.

As used herein, “base editor” as used herein refers to a protein or a fragment thereof having the same catalytic activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest, which in turn can result in a targeted mutation, if the base conversion does not cause a silent mutation, but rather a conversion of an amino acid encoded by the codon comprising the position to be converted with the base editor. At least one base editor according to the present disclosure temporarily or permanently linked to at least one CRISPR-associated effector, or optionally to a component of at least one CRISPR-associated effector complex.

As used herein, the term “Cas9 nuclease” and “Cas9” can be used interchangeably herein, which refer to an RNA-guided DNA endonuclease enzyme associated with the CRISPR (Clustered Regularly Interspaced Short Palindromic Repeats), including the Cas9 protein or fragments thereof (such as a protein comprising an active DNA cleavage domain of Cas9 and/or a gRNA binding domain of Cas9). Cas9 is a component of the CRISPR/Cas genome editing system, which targets and cleaves a DNA target sequence to form a DNA double strand breaks (DSB) under the guidance of a guide RNA.

The term “Cas12 nuclease” and “Cas12” can be used interchangeably herein, which refer to an RNA-guided DNA endonuclease enzyme associated with the CRISPR (Clustered Regularly Interspaced Short Palindromic Repeats), including the Cas12 protein or fragments thereof (such as a protein comprising an active DNA cleavage domain of Cas12 and/or a gRNA binding domain of Cas12). Cas12 is a component of the CRISPR/Cas genome editing system, which targets and cleaves a DNA target sequence to form a DNA double strand breaks (DSB) under the guidance of a guide RNA.

As used herein, a “centimorgan” (cM) is a unit of measure of recombination frequency and genetic distance between two loci. One cM is equal to a 1% chance that a marker at one genetic locus will be separated from a marker at a second locus due to crossing over in a single generation.

As used herein, the terms “clone” or “cloning” when used in the context of plants refers to the production of ‘suckers’ which are genetically identical to the original plant. These suckers can be cut off from the original plant, planted in the ground, and they will grow into a fruit producing plant too.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

As used herein, “closely linked” means that the marker or locus is within about 20 CM, 15 cM, 10 cM, 5 CM, 4 cM, 3 CM, 2 cM, 1 cM, 0.5 CM, or less than 0.5 CM of another marker or locus. For example, 20 cM means that recombination occurs between the marker and the locus with a frequency of equal to or less than about 20%.

As used herein, the term “codon optimization” implies that the codon usage of a DNA or RNA is adapted to that of a cell or organism of interest to improve the transcription rate of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism

As used herein, “complementary” refers to the capacity for pairing, through base stacking and specific hydrogen bonding, between two sequences comprising naturally or non-naturally occurring bases or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and Santa Lucia, Nucl. Acids Research, 2005; 33 (19): 6258-6267.

As referred to herein, a “complementary nucleic acid sequence” is a nucleic acid sequence comprising a sequence of nucleotides that enables it to non-covalently bind to another nucleic acid in a sequence-specific, antiparallel, manner (i.e., a nucleic acid specifically binds to a complementary nucleic acid) under the appropriate in vitro and/or in vivo conditions of temperature and solution ionic strength/

Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters, and MUSCLE (Multiple Sequence Comparison by Log-Expectation; a computer software licensed as public domain).

As used herein, the “control plant” generally refers to a almond plant, plant part, or plant cell that has not been genetically edited. A control plant may be a “null segregant” which is a near isogenic plant that has undergone the same gene editing and regeneration process, but lacks the gene edit(s). A control plant may be “unedited”, referring to a plant which has undergone protoplasting and has subsequently been regenerated from the resulting protoplast. A control plant may also be “wildtype”, referring to a plant that has not undergone the process of protoplasting and regeneration.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

The term “CRISPR RNA” or “crRNA” refers to the RNA strand responsible for hybridizing with target DNA sequences and recruiting CRISPR endonucleases and/or CRISPR-associated effectors. crRNAs may be naturally occurring or may be synthesized according to any known method of producing RNA.

A “CRISPR-associated effector” as used herein can thus be defined as any nuclease, nickase, or recombinase associated with the CRISPR (Clustered Regularly Interspaced Short Palindromic Repeats), having the capacity to introduce a single- or double-strand cleavage into a genomic target site, or having the capacity to introduce a targeted modification, including a point mutation, an insertion, or a deletion, into a genomic target site of interest. At least one CRISPR-associated effector can act on its own, or in combination with other molecules as part of a molecular complex. The CRISPR-associated effector can be present as fusion molecule, or as individual molecules associating by or being associated by at least one of a covalent or non-covalent interaction with gRNA and/or target site so that the components of the CRISPR-associated complex are brought into close physical proximity.

The term “CRISPR landing site” as used herein, refers to a DNA sequence capable of being targeted by a CRISPR-Cas complex. In some embodiments, a CRISPR landing site comprises a proximately placed protospacer/Protospacer Adjacent Motif combination sequence that is capable of being cleaved by a CRISPR complex.

The term “CRISPR complex”, “CRISPR endonuclease complex”, “CRISPR Cas complex”, or “CRISPR-gRNA complex” are used interchangeably herein. “CRISPR complex” refers to a Cas9 nuclease and/or a CRISPR-associated effectors complexed with a guide RNA (gRNA). The term “CRISPR complex” thus refers to a combination of CRISPR endonuclease and guide RNA capable of inducing a double stranded break at a CRISPR landing site. In some embodiments, “CRISPR complex” of the present disclosure refers to a combination of catalytically dead Cas9 protein and guide RNA capable of targeting a target sequence, but not capable of inducing a double stranded break at a CRISPR landing site because it loses a nuclease activity. In other embodiments, “CRISPR complex” of the present disclosure refers to a combination of Cas9 nickase and guide RNA capable of introducing gRNA-targeted single-strand breaks in DNA instead of the double-strand breaks created by wild type Cas enzymes.

As used herein, the term “cross”, “crossing”, “cross pollination” or “crossbreeding” refers to the process by which the pollen of one flower on one plant is applied (artificially or naturally) to the ovule (stigma) of a flower on another plant.

As used herein, the term “deaminase” refers to an enzyme that catalyzes the deamination reaction. In some embodiments of the present disclosure, the deaminase refers to a cytidine deaminase, which catalyzes the deamination of a cytidine or a deoxycytidine to an uracil or a deoxyuridine, respectively. In other embodiments of the present disclosure, the deaminase refers to an adenosine deaminase, which catalyzes the deamination of an adenine to form hypoxanthine (in the form of its nucleoside inosine), which is read as guanine by DNA polymerase.

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

As used herein, the terms “dicotyledon,” “dicot” and “dicotyledonous” refer to a flowering plant having an embryo containing two seed halves or cotyledons. Examples include tobacco; tomato; legumes, including peas, alfalfa, clover and soybeans; oaks; maples; roses; mints; squashes; daisies; walnuts; cacti; violets and buttercups.

As used herein, the term “directing sequence-specific binding” in the context of CRISPR complexes refers to a guide RNA's ability to recruit a CRISPR endonuclease and/or a CRISPR-associated effectors to a CRISPR landing site.

As used herein, “duplex” mutation refers to a genetic modification occurring in two alleles for a given gene.

As used herein, the term “endogenous” or “endogenous gene,” refers to the naturally occurring gene, in the location in which it is naturally found within the host cell genome. “Endogenous gene” is synonymous with “native gene” as used herein. An endogenous gene as described herein can include alleles of naturally occurring genes that have been mutated according to any of the methods of the present disclosure, i.e., an endogenous gene could have been modified at some point by traditional plant breeding methods and/or next generation plant breeding methods.

As used herein, the term “exogenous” refers to a substance coming from some source other than its native source. For example, the terms “exogenous protein,” or “exogenous gene” refer to a protein or gene from a non-native source, and that has been artificially supplied to a biological system. As used herein, the term “exogenous” is used interchangeably with the term “heterologous,” and refers to a substance coming from some source other than its native source.

As used herein, “expression” and “expression level” refer to the relative or absolute amount of a functional gene product present in a cell. As used herein, “gene products” include, but are not limited to, nucleic acids (e.g. RNA), post-transcriptionally modified nucleic acids (e.g. spliced RNA, poly-adenylated mRNA), proteins (e.g. enzymes, structural proteins, etc.), and post-translationally modified proteins (e.g. glycoproteins, lipoproteins, etc.). The function of the gene product refers to the wild-type, unmodified, uninhibited function of the gene product. As used herein, “decreased expression” refers to a relative decrease in the amount of a functional gene product present in a cell. The decreased expression may refer to a decrease in the total amount of a gene product present in a cell (e.g. a decrease in the amount of a protein) or to a decrease in the amount of functional gene products present in a cell (e.g. a decrease in the percentage of proteins with wild-type function, e.g., an altered activity of the protein) or to a decrease in the function of gene products present in a cell (e.g. a decrease in the activity of proteins as compared to proteins with wild-type function). The decreased expression may be of a gene product encoded at a certain genomic locus. Decreased expression also includes “non-expression.” As used herein, “non-expression” refers to the absence of a functional gene product present in a cell, or to an expression level insufficient for detection of the gene product in the cell, or to an expression level insufficient to result in the function of the gene product within the cell, or to an activity level insufficient to result in the detectable activity of the gene product within the cell.

As used herein, “expression cassette” refers to DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development in animal and/or plant including almond species.

As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The term “gene edited plant, part or cell” as used herein refers to a plant, part or cell that comprises one or more endogenous genes that are edited by a gene editing system. The gene editing system of the present disclosure comprises a targeting element and/or an editing element. The targeting element can recognize a target genomic sequence. The editing element can modify the target genomic sequence, e.g., by substitution or insertion of one or more nucleotides in the genomic sequence, deletion of one or more nucleotides in the genomic sequence, alteration of genomic sequences to include regulatory sequences, insertion of transgenes at a safe harbor genomic site or other specific location in the genome, or any combination thereof. The targeting element and the editing element can be on the same nucleic acid molecule or different nucleic acid molecules. In some embodiments, the editing element is capable of precise genome editing by substitution of a single nucleotide using a base editor, such cytosine base editor (CBE) and/or adenine base editor (ABE), which is directly or indirectly fused to a CRISPR-associated effector protein.

As used herein, “genetic modification” or “modification” refers to any sequence or portion thereof within a nucleic acid molecule that differs from the sequence of an ancestral nucleic acid molecule. For example, a seed that contains an inserted or deleted genomic sequence that is not present in one of its parent plants comprises a genetic modification. A genetic modification may be naturally occurring or introduced. A genetic modification may be introduced via, for example: plant breeding to introduce a naturally-occurring genetic modification of one plant line into another plant line; transgenic methods; gene editing; chemical mutagenesis; and the like.

As used herein, “genotype” is the genetic constitution of an individual (or group of individuals) at one or more genetic loci, as contrasted with the observable trait (phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome. The term “genotype” can also refer to determining the genetic constitution of an individual (or group of individuals) at one or more genetic loci.

As used herein, “germplasm” refers to living sources of genetic material. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed, or tissues from which new plants can be grown, or plant parts, such as leaves, stems, pollen, or cells that can be cultured into a whole plant.

The terms “growing”, or “regeneration” as used herein mean growing a whole, differentiated plant from a plant cell, a group of plant cells, a plant part (including seeds), or a plant piece (e.g., from a protoplast, callus, or tissue part).

The term “Guided endonuclease” refers to a polypeptide having RNA binding activity, DNA binding activity, and/or DNA cleavage activity. RNA guided endonucleases form a complex with a guide RNA, which contains a sequence that is able to bind a target sequence on double stranded DNA. In some embodiments, the RNA guided endonuclease cleaves the double stranded target DNA.

The term “Guide RNA” or “gRNA” as used herein refers to an RNA sequence or combination of sequences capable of recruiting a CRISPR endonuclease and/or CRISPR-associated effectors to a target sequence. Typically, gRNA is composed of crRNA and tracrRNA molecules forming complexes through partial complement, wherein crRNA comprises a sequence that is sufficiently complementary to a target sequence for hybridization and directs the CRISPR complex (i.e., Cas9-crRNA/tracrRNA hybrid) to specifically bind to the target sequence. Also, single guide RNA (sgRNA) can be designed, which comprises the characteristics of both crRNA and tracrRNA. Therefore, as used herein, a guide RNA can be a natural or synthetic crRNA (e.g., for Cpf1), a natural or synthetic crRNA/tracrRNA hybrid (e.g., for Cas9), or a single-guide RNA (sgRNA).

The term “guide sequence” or “spacer sequence” refers to the portion of a crRNA or guide RNA (gRNA) that is responsible for hybridizing with the target DNA.

As used herein, the term “heterologous” refers to a substance coming from some source or location other than its native source or location. In some embodiments, the term “heterologous nucleic acid” refers to a nucleic acid sequence that is not naturally found in a particular organism. For example, the term “heterologous promoter” may refer to a promoter that has been taken from one source organism and utilized in another organism, in which the promoter is not naturally found. However, the term “heterologous promoter” may also refer to a promoter that is from within the same source organism, but has merely been moved to a novel location, in which said promoter is not normally located.

Heterologous gene sequences can be introduced into a target cell by using an “expression vector,” which can be a eukaryotic expression vector, for example a plant expression vector. Methods used to construct vectors are well known to a person skilled in the art and described in various publications. In particular, techniques for constructing suitable vectors, including a description of the functional components such as promoters, enhancers, termination and polyadenylation signals, selection markers, origins of replication, and splicing signals, are reviewed in the prior art. Vectors may include but are not limited to plasmid vectors, phagemids, cosmids, artificial/mini-chromosomes (e.g. ACE), or viral vectors such as baculovirus, retrovirus, adenovirus, adeno-associated virus, herpes simplex virus, retroviruses, bacteriophages. The eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operatively linked, are well known in the art and some are commercially available from companies such as Stratagene, La Jolla, Calif.; Invitrogen, Carlsbad, Calif.; Promega, Madison, Wis. or BD Biosciences Clontech, Palo Alto, Calif. In one embodiment the expression vector comprises at least one nucleic acid sequence which is a regulatory sequence necessary for transcription and translation of nucleotide sequences that encode for a peptide/polypeptide/protein of interest.

As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

As used herein, the term “homologous” or “homolog” is known in the art and refers to related sequences that share a common ancestor or family member and are determined based on the degree of sequence identity. The terms “homology”, “homologous”, “substantially similar” and “corresponding substantially” are used interchangeably herein. Homologs usually control, mediate, or influence the same or similar biochemical pathways, yet particular homologs may give rise to differing phenotypes. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared.

As used herein, the term “heterozygote” refers to a diploid or polyploid individual cell or plant having different alleles (forms of a given gene) present at least at one locus.

As used herein, the term “heterozygous” refers to the presence of different alleles (forms of a given gene) at a particular gene locus.

As used herein, the term “homozygote” refers to an individual cell or plant having the same alleles at one or more loci.

As used herein, the term “homozygous” refers to the presence of identical alleles at one or more loci in homologous chromosomal segments.

The term “homolog” is sometimes used to apply to the relationship between genes separated by the event of speciation (see “ortholog”) or to the relationship between genes separated by the event of genetic duplication (see “paralog”).

The term “homeolog” refers to a homoeologous gene or chromosome, resulting from polyploidy or chromosomal duplication events. This contrasts with the more common ‘homolog’, which is defined immediately above.

“Homologous sequences” or “homologs” or “orthologs” are thought, believed, or known to be functionally related. A functional relationship may be indicated in any one of several ways, including, but not limited to: (a) degree of sequence identity and/or (b) the same or similar biological function. Preferably, both (a) and (b) are indicated. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Michigan), AlignX, and Vector NTI (Invitrogen, Carlsbad, CA).

The terms “host cell”, “genetically engineered host cell,” “recombinant host cell,” and “recombinant strain” are used interchangeably herein and refer to host cells that have been genetically engineered by the methods of the present disclosure. Thus, the terms include a host cell (e.g., bacteria, yeast cell, fungal cell, CHO, human cell, plant cell, protoplast derived from plant, callus, etc.) that has been genetically altered, modified, or engineered, such that it exhibits an altered, modified, or different genotype and/or phenotype (e.g., when the genetic modification affects coding nucleic acid sequences), as compared to the naturally-occurring host cell from which it was derived. It is understood that the terms refer not only to the particular recombinant host cell in question, but also to the progeny or potential progeny of such a host cell.

As used herein, the term “hybridize” refers to pairing between complementary nucleotide bases (e.g., adenine (A) forms a base pair with thymine (T) in a DNA molecule and with uracil (U) in an RNA molecule, and guanine (G) forms a base pair with cytosine (C) in both DNA and RNA molecules) to form a double-stranded nucleic acid molecule. (See, e.g., Wahl and Berger (1987) Methods Enzymol. 152:399; Kimmel, (1987) Methods Enzymol. 152:507). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to an uracil (U), and vice versa. As such, when a G/U base-pair can be made at a given nucleotide position a protein-binding segment (dsRNA duplex) of a guide RNA molecule, the position is not considered to be non-complementary but is instead considered to be complementary. It is understood in the art that the sequence of polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted.

As used herein, the terms “introgression”, “introgressed” and “introgressing” refer to the process whereby genes of one species, variety or cultivar are moved into the genome of another species, variety or cultivar, by crossing those species. The crossing may be natural or artificial. The process may optionally be completed by backcrossing to the recurrent parent, in which case introgression refers to infiltration of the genes of one species into the gene pool of another through repeated backcrossing of an interspecific hybrid with one of its parents. An introgression may also be described as a heterologous genetic material stably integrated in the genome of a recipient plant.

The disclosure encompasses isolated or substantially purified nucleic acid or protein compositions. As used herein, an “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified polynucleotide or polypeptide is substantially free of other cellular material, or culture medium when produced by recombinant techniques, or substantially free of chemical precursors or other chemicals when chemically synthesized. Suitably, an “isolated” polynucleotide is free of sequences (especially protein encoding sequences) that naturally flank the polynucleotide (i.e., sequences located at the 5′ and 3′ ends of the polynucleotide) in the genomic DNA of the organism from which the polynucleotide was derived. For example, in various embodiments, the isolated polynucleotide can contain less than about 5 kb, 4 kb, 3 kb, 2 kb, 1 kb, 0.5 kb, or 0.1 kb of nucleotide sequences that naturally flank the polynucleotide in genomic DNA of the cell from which the polynucleotide was derived. A polypeptide that is substantially free of cellular material includes preparations of protein having less than about 30%, 20%, 10%, 5%, (by dry weight) of contaminating protein. When the protein of the disclosure or biologically active portion thereof is recombinantly produced, culture medium suitably represents less than about 30%, 20%, 10%, or 5% (by dry weight) of chemical precursors or non-protein-of-interest chemicals.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g., in terms of the sexual crosses affected such that a gene or a combination of genes, in heterozygous (hemizygous) or homozygous condition, imparts a desired trait to the plant.

As used herein, the term “locus” (plural: “loci”) refers to any site that has been defined genetically. A locus may be a gene, or part of a gene, or a DNA sequence that has some regulatory role, and may be occupied by different sequences.

As used herein, the term “mass selection” refers to a form of selection in which individual plants are selected and the next generation propagated from the aggregate of their seeds. More details of mass selection are described herein in the specification.

The term “modified” refers to a substance or compound (e.g., a cell, a polynucleotide sequence, and/or a polypeptide sequence) that has been altered or changed as compared to the corresponding unmodified substance or compound.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed quite easily by the average person skilled in molecular-biological techniques which techniques are for instance described in Lefebvre and Chevre, 1995, Lorez and Wenzel, 2007, Srivastava and Narula, 2004, Meksem and Kahl, 2005, and Phillips and Vasil, 2001. General information concerning AFLP technology can be found in Vos et al. (1995, AFLP: a new technique for DNA fingerprinting, Nucleic Acids Res. 1995 Nov. 11; 23(21): 4407-4414).

A probe comprises an identifiable, isolated nucleic acid that recognizes a target nucleic acid sequence. A probe includes a nucleic acid that is attached to an addressable location, a detectable label or other reporter molecule and that hybridizes to a target sequence. Typical labels include radioactive isotopes, enzyme substrates, co-factors, ligands, chemiluminescent or fluorescent agents, haptens, and enzymes. Methods for labelling and guidance in the choice of labels appropriate for various purposes are discussed, for example, in Sambrook et al. (ed.), Molecular Cloning: A Laboratory Manual, 2^{nd} ed., vol. 1-3, Cold Spring Harbor Laboratory Press, Cold Spring Harbor, NY, 1989 and Ausubel et al. Short Protocols in Molecular Biology, 4^{th} ed., John Wiley & Sons, Inc., 1999.

As used herein, “mutagenesis” refers to processes in which mutations are introduced into a selected DNA sequence. Mutations induced by endonucleases generally are obtained by a double strand break, which results in insertion/deletion mutations (“indels”) that can be detected by deep-sequencing analysis. Such mutations typically are deletions of several base pairs, and have the effect of inactivating the mutated allele. In the methods described herein, for example, mutagenesis occurs via double stranded DNA breaks made by TALE-nucleases targeted to selected DNA sequences in a plant cell. Such mutagenesis results in “TALE-nuclease-induced mutations” (e.g., TALE-nuclease-induced knockouts) and reduced expression of the targeted gene. Following mutagenesis, plants can be regenerated from the treated cells using known techniques (e.g., planting seeds in accordance with conventional growing procedures, followed by self-pollination).

As used herein, the term “naturally occurring” as applied to a nucleic acid, a polypeptide, a cell, or an organism, refers to a nucleic acid, polypeptide, cell, or organism that is found in nature. The term “naturally occurring” may refer to a gene or sequence derived from a naturally occurring source. Thus, for the purposes of this disclosure, a “non-naturally occurring” sequence is a sequence that has been synthesized, mutated, engineered, edited, or otherwise modified to have a different sequence from known natural sequences. In some embodiments, the modification may be at the protein level (e.g., amino acid substitutions). In other embodiments, the modification may be at the DNA level (e.g., nucleotide substitutions).

As used herein, the term “non-regenerable” generally refers to an almond plant part, a plant cell, a processed almond product, or a portion or cell of any of the foregoing, that cannot be induced to form a whole almond plant or that cannot be induced to form a whole almond plant that is capable of sexual and/or asexual reproduction.

As used herein, the term “nucleotide change” or “nucleotide modification” refers to, e.g., nucleotide substitution, deletion, and/or insertion, as is well understood in the art. For example, such nucleotide changes/modifications include mutations containing alterations that produce silent substitutions, additions, or deletions, but do not alter the properties or activities of the encoded protein or how the proteins are made. As another example, such nucleotide changes/modifications include mutations containing alterations that produce replacement substitutions, additions, or deletions, that alter the properties or activities of the encoded protein or how the proteins are made.

As used herein, the term “offspring” refers to any plant resulting as progeny from a vegetative or sexual reproduction from one or more parent plants or descendants thereof. For instance, an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true breeding is homozygous for a trait), while an F2 may be (and usually is) an offspring resulting from self-pollination of said F1 hybrids.

As used herein, the term “open pollination” refers to a plant population that is freely exposed to some gene flow, as opposed to a closed one in which there is an effective barrier to gene flow.

As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

The term “ortholog” refers to genes in different species that evolved from a common ancestral gene by speciation. Normally, orthologs retain the same function in the course of evolution. Identification of orthologs is critical for reliable prediction of gene function in newly sequenced genomes.

As used herein when discussing plants, the term “ovule” refers to the female gametophyte, whereas the term “pollen” means the male gametophyte.

The term “paralog” refers to genes related by duplication within a genome. While orthologs generally retain the same function in the course of evolution, paralogs can evolve new functions, even if these are related to the original one.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “phenotype” refers to the observable characters of an individual cell, cell culture, organism (e.g., a plant), or group of organisms which results from the interaction between that individual's genetic makeup (i.e., genotype) and the environment.

The term “plant” refers to a whole plant, any part thereof, or a cell or tissue culture derived from a plant, comprising any of: whole plants, plant components or organs (e.g., leaves, stems, roots, etc.), plant tissues, seeds, embryos, plant cells, protoplasts and/or progeny of the same. A plant cell is a biological cell of a plant, taken from a plant or derived through culture of a cell taken from a plant.

The term “plant part” includes differentiated and undifferentiated tissues including, but not limited to plant organs, plant tissues, roots, stems, shoots, rootstocks, scions, stipules, petals, leaves, flowers, ovules, pollens, bracts, petioles, internodes, bark, pubescence, tillers, rhizomes, fronds, blades, stamens, fruits, seeds, tumor tissue and plant cells (e.g., single cells, protoplasts, embryos, and callus tissue). Plant cells include, without limitation, cells from seeds, suspension cultures, embryos, meristematic regions, callus tissue, leaves, roots, shoots, gametophytes, sporophytes, pollen and microspores. The plant tissue may be in a plant or in a plant organ, tissue or cell culture.

As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

The terms “polynucleotide,” “nucleic acid,” and “nucleotide sequence,” used interchangeably herein, refer to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. This term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. It also includes modified nucleic acids such as methylated and/or capped nucleic acids, nucleic acids containing modified bases, backbone modifications, and the like. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” “nucleic acid,” and “nucleotide sequence” should be understood to include, as applicable to the embodiments being described, single-stranded (such as sense or antisense) and double-stranded polynucleotides.

As used herein, the term “population” means a genetically homogeneous or heterogeneous collection of plants sharing a common genetic derivation.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T and G/C content) of the primer. A pair of bi-directional primers consists of one forward and one reverse primer as commonly used in the art of DNA amplification such as in PCR amplification.

The term “protospacer” refers to the DNA sequence targeted by a guide sequence of crRNA or gRNA. In some embodiments, the protospacer sequence hybridizes with the crRNA or gRNA guide (spacer) sequence of a CRISPR complex.

As used herein, “quadraplex” mutation refers to a genetic modification occurring in four alleles for a given gene.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is aware of the genetic elements that must be present on the vector to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, which replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, which is not autonomously replicating. As used herein, the term “expression” refers to the production of a functional end-product e.g., an mRNA or a protein (precursor or mature).

The term “seed region” refers to the critical portion of a crRNA's or guide RNA's guide sequence that is most susceptible to mismatches with their targets. In some embodiments, a single mismatch in the seed region of a crRNA/gRNA can render a CRISPR complex inactive at that binding site. In some embodiments, the seed regions for Cas9 endonucleases are located along the last ~12 nts of the 3′ portion of the guide sequence, which correspond (hybridize) to the portion of the protospacer target sequence that is adjacent to the PAM. In some embodiments, the seed regions for Cpf1 endonucleases are located along the first ~5 nts of the 5′ portion of the guide sequence, which correspond (hybridize) to the portion of the protospacer target sequence adjacent to the PAM.

As used herein, the term “self-crossing”, “self-pollinated” or “self-pollination” means the pollen of one flower on one plant is applied (artificially or naturally) to the ovule (stigma) of the same or a different flower on the same plant.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988).

As used herein, “simplex” mutation refers to a genetic modification occurring in only one allele for a given gene.

The term “single allele converted plant” as used herein refers to those plants which are developed by a plant breeding technique called backcrossing wherein essentially all of the desired morphological and physiological characteristics of an inbred are recovered in addition to the single allele transferred into the inbred via the backcrossing technique.

As used herein the term “targeted” refers to the expectation that one item or molecule will interact with another item or molecule with a degree of specificity, so as to exclude non-targeted items or molecules. For example, a first polynucleotide that is targeted to a second polynucleotide, according to the present disclosure has been designed to hybridize with the second polynucleotide in a sequence specific manner (e.g., via Watson-Crick base pairing). In some embodiments, the selected region of hybridization is designed so as to render the hybridization unique to the one, or more targeted regions. A second polynucleotide can cease to be a target of a first targeting polynucleotide, if its targeting sequence (region of hybridization) is mutated or is otherwise removed/separated from the second polynucleotide. Furthermore, “targeted” can be interchangeably used with “site-specific” or “site-directed,” which refers to an action of molecular biology which uses information on the sequence of a genomic region of interest to be modified, and which further relies on information of the mechanism of action of molecular tools, e.g., nucleases, including CRISPR nucleases and variants thereof, TALENs, ZFNs, meganucleases or recombinases, DNA-modifying enzymes, including base modifying enzymes like cytidine deaminase enzymes, histone modifying enzymes and the like, DNA-binding proteins, cr/tracr RNAs, guide RNAs and the like.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, leaves, stems, roots, root tips, anthers, pistils, meristematic cells, axillary buds, ovaries, seed coat, endosperm, hypocotyls, cotyledons and the like. The term “plant organ” refers to plant tissue or a group of tissues that constitute a morphologically and functionally distinct part of a plant. “Progeny” comprises any subsequent generation of a plant.

The term “tracrRNA” refers to a small trans-encoded RNA. TracrRNA is complementary to and base pairs with crRNA to form a crRNA/tracrRNA hybrid, capable of recruiting CRISPR endonucleases and/or CRISPR-associated effectors to target sequences.

The terms “transgene” or “transgenic” as used herein refer to at least one nucleic acid sequence that is taken from the genome of one organism, or produced synthetically, and which is then introduced into a host cell or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation and thus inheritance of the respective at least one molecule introduced into the genome of a cell. The terms “transgene-free” refers to a condition that transgene is not present or found in the genome of a host cell or tissue or organism of interest.

As used herein, “triplex” mutation refers to a genetic modification occurring in three alleles for a given gene.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein.

Variant proteins encompassed by the present disclosure are biologically active, that is they continue to possess the desired biological activity of the native protein, that is, modulating or regulatory activity as described herein. Such variants may result from, for example, genetic polymorphism or from human manipulation. Biologically active variants of a native R protein of the disclosure will have at least 40%, 50%, 60%, 70%, generally at least 75%, 80%, 85%, preferably about 90% to 95% or more, and more preferably about 98% or more sequence identity to the amino acid sequence for the native protein as determined by sequence alignment programs described elsewhere herein using default parameters. A biologically active variant of a protein of the disclosure may differ from that protein by as few as 1-15 amino acid residues, as few as 1-10, such as 6-10, as few as 5, as few as 4, 3, 2, or even 1 amino acid residue.

The nucleic proteins of the disclosure may be altered in various ways including amino acid substitutions, deletions, truncations, and insertions. Methods for such manipulations are generally known in the art. For example, amino acid sequence variants of the R proteins can be prepared by mutations in the DNA. Methods for mutagenesis and nucleotide sequence alterations are well known in art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

Individual substitutions, deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another, Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine I, Lysine (K), Histidine (H); and Acidic: Aspartic acid (D), Glutamic acid (E), Asparagine (N), Glutamine (Q). See also, Creighton, 1984. In addition, individual substitutions, deletions or additions which alter, add or delete a single amino acid or a small percentage of amino acids in an encoded sequence are also “conservatively modified variations.”

Methods for preparing and using nucleic acid probes and primers are described, for example, in Sambrook et al. (ed.), Molecular Cloning: A Laboratory Manual, 2^{nd} ed., vol. 1-3, Cold Spring Harbor Laboratory Press, Cold Spring Harbor, NY, 1989; Ausubel et al. Short Protocols in Molecular Biology, 4^{th} ed., John Wiley & Sons, Inc., 1999; and Innis et al. PCR Protocols, A Guide to Methods and Applications, Academic Press, Inc., San Diego, CA, 1990. Amplification primer pairs can be derived from a known sequence, for example, by using computer programs intended for that purpose such as PRIMER (Version 0.5, 1991, Whitehead Institute for Biomedical Research, Cambridge, MA). One of ordinary skill in the art will appreciate that the specificity of a particular probe or primer increases with its length. Thus, to obtain greater specificity, probes and primers can be selected that comprise at least 20, 25, 30, 35, 40, 45, 50 or more consecutive nucleotides of a target nucleotide sequence. For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3rd ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially mismatched primers, and the like.

As used herein, the term “variety” or “cultivar” means a group of similar plants that by structural features and performance can be identified from other varieties within the same species. The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

As used herein, the term “vector”, “plasmid”, or “construct” refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, recombinant plant viruses. Non-limiting examples of plant viruses include, TMV-mediated (transient) transfection into tobacco (Tuipe, TH et al (1993), J. Virology Meth, 42:227-239), ssDNA genomes viruses (e.g., family Geminiviridae), reverse transcribing viruses (e.g., families Caulimoviridae, Pseudoviridae, and Metaviridae), dsNRA viruses (e.g., families Reoviridae and Partitiviridae), (−) ssRNA viruses (e.g., families Rhabdoviridae and Bunyaviridae), (+) ssRNA viruses (e.g., families Bromoviridae, Closteroviridae, Comoviridae, Luteoviridae, Potyviridae, Sequiviridae and Tombusviridae) and viroids (e.g., families Pospiviroldae and Avsunviroidae). Detailed classification information of plant viruses can be found in Fauquet et al (2008, “Geminivirus strain demarcation and nomenclature”. Archives of Virology 153:783-821, incorporated herein by reference in its entirety), and Khan et al. (Plant viruses as molecular pathogens; Publisher Routledge, 2002, ISBN 1560228954, 9781560228950). Examples of non-viral vectors include, but are not limited to, liposomes, polyamine derivatives of DNA, and the like.

Also, “vector” is defined to include, inter alia, any plasmid, cosmid, phage or Agrobacterium binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

### BRIEF DESCRIPTION

In some embodiments, the present disclosure describes a methods and systems for the production of a self-fertile almond plant (Prunus dulcis) with one or more S-locus haplotypes, wherein one or more genes within the one or more S-locus haplotypes have a deletion as compared to the respective sequence of the one or more parent genes. As used herein, “parent genes” refer to the wild-type genes present in the S-locus of a self-incompatible parent variety. For example, in some embodiments, the parent genes are the wild-type genes present in the S-locus of the ‘Nonpareil’ variety. For example, in some embodiments, the parent genes are the wild-type genes present in the S-locus of the ‘Carmel’ variety.

The deletion can be any size and can be made using any method known to be effective for creating deletions in a nucleotide sequence. In some embodiments, the deletion is achieved with a guided nuclease. In some embodiments, the deletion is achieved with a site-specific nuclease. In some embodiments, the deletion is between about 1 to 35 base pairs (“bp”) in length. In some embodiments, the deletion is between about 2 to 20 bp in length. In certain embodiments, the deletion results in a frameshift mutation or an in-frame mutation. In some embodiments, the deletion is 1 bp, 2 bp, 3 bp, 4 bp, 5 bp, 6, bp, 7 bp, 8 bp, 9 bp, 10 bp, 11 bp, 13 bp, 14 bp, 16 bp, 17 bp, 19 bp, 20 bp, 22 bp, 23 bp, 25 bp, 26 bp, 28 bp, 29, 31 bp, 32 bp, 34 bp or 35 bp in length. In some embodiments, the one or more deletions substantially prevent expression of one or more S-locus gene products, resulting in a self-fertile almond plant.

In some embodiments, the self-fertile almond plant includes an S-locus with an “S8 haplotype” (hereinafter the “S8-locus”), wherein one or more genes within the S8-locus have a deletion as compared to the respective sequence of the one or more parent genes.

In some embodiments, the method for the production of a self-fertile almond plant includes providing an S-locus with an “S8 haplotype” (hereinafter the “S8-locus”), wherein one or more genes within the S8-locus have a deletion as compared to the respective sequence of the one or more parent genes.

In some embodiments, the S8-locus includes a gene encoding an S-RNase protein (hereinafter “S8-RNase”). The transcribed portion of the S8-RNase gene (including both exons and introns, i.e., the “pre-mRNA sequence”) is provided by SEQ ID NO: 1. The protein coding sequence for S8-RNase (i.e., exons only) is provided by SEQ ID NO: 2. The amino acid sequence of the S8-RNase protein is provided by SEQ ID NO: 3.

In some embodiments, the S8-locus includes a gene encoding an SFB protein or S haplotype-specific F-box protein, (hereinafter “S8-SFB”). The transcribed portion of the S8-SFB gene is provided by SEQ ID NO: 4. There are no introns present in the S8-SFB gene, therefore the transcribed sequence is the same sequence as the protein coding sequence. The amino acid sequence of the S8-SFB protein is provided by SEQ ID NO: 5.

In some embodiments, the self-fertile almond plant includes an S-locus with the “S7 haplotype” (hereinafter the “S7-locus”), wherein one or more genes within the S7-locus have a deletion as compared to the respective sequence of the one or more parent genes.

In some embodiments, the method for the production of a self-fertile almond plant includes providing an S-locus with the “S7 haplotype” (hereinafter the “S7-locus”), wherein one or more genes within the S7-locus have a deletion as compared to the respective sequence of the one or more parent genes.

In some embodiments, the S7-locus includes a gene encoding an S-RNase protein (hereinafter “S7-RNase”). The transcribed portion of the S7-RNase gene (including both exons and introns, i.e., the “pre-mRNA sequence”) is provided by SEQ ID NO: 6. The protein coding sequence for S7-RNase (i.e., exons only) is provided by SEQ ID NO: 7. The amino acid sequence of the S7-RNase protein is provided by SEQ ID NO: 8.

In some embodiments, the S7-locus includes a gene encoding an SFB protein (hereinafter “S7-SFB”). The transcribed portion of the S7-SFB gene is provided by SEQ ID NO: 9. There are no introns present in the S7-SFB gene, therefore the transcribed sequence is the same sequence as the protein coding sequence. The amino acid sequence of the S7-SFB protein is provided by SEQ ID NO: 10.

In some embodiments, the self-fertile almond plant includes both the S8-locus and the S7-locus, wherein one or more genes within the S8-locus and/or the S7-locus have a deletion as compared to the respective sequence of the one or more parent genes. In some embodiments, the parent variety of the self-fertile almond tree is ‘Nonpareil’ (2n=S8/S7).

In some embodiments, the method for the production of a self-fertile almond plant includes providing both the S8-locus and the S7-locus, wherein one or more genes within the S8-locus and/or the S7-locus have a deletion as compared to the respective sequence of the one or more parent genes. In some embodiments, the parent variety of the self-fertile almond tree is ‘Nonpareil’ (2n=S8/S7).

In some embodiments, the self-fertile almond plant includes an S-locus with the “S5 haplotype” (hereinafter the “S5-locus”), wherein one or more genes within the S5-locus have a deletion as compared to the respective sequence of the one or more parent genes.

In some embodiments, the method for the production of a self-fertile almond plant includes providing an S-locus with the “S5 haplotype” (hereinafter the “S5-locus”), wherein one or more genes within the S5-locus have a deletion as compared to the respective sequence of the one or more parent genes.

In some embodiments, the S5-locus includes a gene encoding an S-RNase protein (hereinafter “S5-RNase”). In some embodiments, the S5-locus includes a gene encoding an SFB protein (hereinafter “S5-SFB”).

In some embodiments, the self-fertile almond plant includes both the S8-locus and the S5-locus, wherein one or more genes within the S8-locus and/or the S5-locus have a deletion as compared to the respective sequence of the one or more parent genes. In some embodiments, the parent variety of the self-fertile almond tree is Carmel (2n=S8/S5).

In some embodiments, the method for the production of a self-fertile almond plant includes providing both the S8-locus and the S5-locus, wherein one or more genes within the S8-locus and/or the S5-locus have a deletion as compared to the respective sequence of the one or more parent genes. In some embodiments, the parent variety of the self-fertile almond tree is Carmel (2n=S8/S5).

In some embodiments, a guided nuclease is used to make a deletion in the S8-RNase gene, wherein the guided nuclease is directed to a target site by a guide RNA. In some embodiments, the guide RNA includes an RNA sequence with at least 90%, at least 95%, at least 99% or 100% sequence identity to SEQ ID NO: 17. In some embodiments, the guide RNA includes an RNA sequence with at least 90%, at least 95%, at least 99% or 100% sequence identity to SEQ ID NO: 18. In some embodiments, the target site in the S8-RNase gene comprises a DNA sequence with at least 90%, at least 95%, at least 99% or 100% sequence identity to SEQ ID NO: 37. In some embodiments, the target site in the S8-RNase gene comprises a DNA sequence with at least 90%, at least 95%, at least 99% or 100% sequence identity to SEQ ID NO: 38.

In some embodiments, the method for the production of a self-fertile almond plant including providing a guided nuclease is used to make a deletion in the S8-RNase gene, wherein the guided nuclease is directed to a target site by a guide RNA. In some embodiments, the guide RNA includes an RNA sequence with at least 90%, at least 95%, at least 99% or 100% sequence identity to SEQ ID NO: 17. In some embodiments, the guide RNA includes an RNA sequence with at least 90%, at least 95%, at least 99% or 100% sequence identity to SEQ ID NO: 18. In some embodiments, the target site in the S8-RNase gene comprises a DNA sequence with at least 90%, at least 95%, at least 99% or 100% sequence identity to SEQ ID NO: 37. In some embodiments, the target site in the S8-RNase gene comprises a DNA sequence with at least 90%, at least 95%, at least 99% or 100% sequence identity to SEQ ID NO: 38.

In some embodiments, the self-fertile almond plant includes an S8-RNase gene, wherein a portion of the S8-RNase gene has at least 90%, at least 95%, at least 99% or 100% sequence identity to SEQ ID NO: 1, except for a deletion (as compared to the respective, unedited S8-RNase gene) initiated by a guided nuclease in a region between about 20 bp upstream, and about 20 bp downstream (i.e., about +/−20 bp) from the 3′ end of a target site in the S8-RNase gene. In some embodiments, the guided nuclease has a guide RNA which includes an RNA sequence with at least 90%, at least 95%, at least 99% or 100% sequence identity to SEQ ID NO: 17. In some embodiments, the guide RNA includes an RNA sequence with at least 90%, at least 95%, at least 99% or 100% sequence identity to SEQ ID NO: 18. In some embodiments, the target site in the S8-RNase gene comprises a DNA sequence with at least 90%, at least 95%, at least 99% or 100% sequence identity to SEQ ID NO: 37. In some embodiments, the target site in the S8-RNase gene comprises a DNA sequence with at least 90%, at least 95%, at least 99% or 100% sequence identity to SEQ ID NO: 38.

In some embodiments, the method for the production of a self-fertile almond plant includes providing an S8-RNase gene, wherein a portion of the S8-RNase gene has at least 90%, at least 95%, at least 99% or 100% sequence identity to SEQ ID NO: 1, except for a deletion (as compared to the respective, unedited S8-RNase gene) initiated by a guided nuclease in a region between about 20 bp upstream, and about 20 bp downstream (i.e., about +/−20 bp) from the 3′ end of a target site in the S8-RNase gene. For clarity, this terminology is meant to describe the physical location of a deletion within the target gene, not the size of the deletion itself, such that for a deletion to occur “about +/−20 bp from the 3′ end” of a target site, the position of at least one of the deleted base pairs must be located within 20 bp of the 3′ end of a target site. In some embodiments, the guided nuclease has a guide RNA which includes an RNA sequence with at least 90%, at least 95%, at least 99% or 100% sequence identity to SEQ ID NO: 17. In some embodiments, the guide RNA includes an RNA sequence with at least 90%, at least 95%, at least 99% or 100% sequence identity to SEQ ID NO: 18. In some embodiments, the target site in the S8-RNase gene comprises a DNA sequence with at least 90%, at least 95%, at least 99% or 100% sequence identity to SEQ ID NO: 37. In some embodiments, the target site in the S8-RNase gene comprises a DNA sequence with at least 90%, at least 95%, at least 99% or 100% sequence identity to SEQ ID NO: 38.

In some embodiments, a guided nuclease is used to make a deletion in the S8-SFB gene, wherein the guided nuclease is directed to a target site by a guide RNA. In some embodiments, the guide RNA includes an RNA sequence with at least 90%, at least 95%, at least 99% or 100% sequence identity to SEQ ID NO: 57. In some embodiments, the target site in the S8-SFB gene comprises a DNA sequence with at least 90%, at least 95%, at least 99% or 100% sequence identity to SEQ ID NO: 70.

In some embodiments, the method for the production of a self-fertile almond cultivar comprises providing a guided nuclease that is used to make a deletion in the S8-SFB gene, wherein the guided nuclease is directed to a target site by a guide RNA. In some embodiments, the guide RNA includes an RNA sequence with at least 90%, at least 95%, at least 99% or 100% sequence identity to SEQ ID NO: 57. In some embodiments, the target site in the S8-SFB gene comprises a DNA sequence with at least 90%, at least 95%, at least 99% or 100% sequence identity to SEQ ID NO: 70.

In some embodiments, the self-fertile almond plant includes an S8-SFB gene, wherein a portion of the S8-SFB gene has at least 90%, at least 95%, at least 99% or 100% sequence identity to SEQ ID NO: 4, except for a deletion (as compared to the respective, unedited S8-SFB gene) initiated by a guided nuclease in a region between about 20 bp upstream, and about 20 bp downstream (i.e., about +/−20 bp) from the 3′ end of a target site in the S8-SFB gene. In some embodiments, the guided nuclease has a guide RNA which includes an RNA sequence with at least 90%, at least 95%, at least 99% or 100% sequence identity to SEQ ID NO: 57. In some embodiments, the target site in the S8-SFB gene comprises a DNA sequence with at least 90%, at least 95%, at least 99% or 100% sequence identity to SEQ ID NO: 70.

In some embodiments, the method for the production of a self-fertile almond plant includes providing an S8-SFB gene, wherein a portion of the S8-SFB gene has at least 90%, at least 95%, at least 99% or 100% sequence identity to SEQ ID NO: 4, except for a deletion (as compared to the respective, unedited S8-SFB gene) initiated by a guided nuclease in a region between about 20 bp upstream, and about 20 bp downstream (i.e., about +/−20 bp) from the 3′ end of a target site in the S8-SFB gene. In some embodiments, the guided nuclease has a guide RNA which includes an RNA sequence with at least 90%, at least 95%, at least 99% or 100% sequence identity to SEQ ID NO: 57. In some embodiments, the target site in the S8-SFB gene comprises a DNA sequence with at least 90%, at least 95%, at least 99% or 100% sequence identity to SEQ ID NO: 70.

In some embodiments, a guided nuclease is used to make a deletion in the S7-RNase gene, wherein the guided nuclease is directed to a target site by a guide RNA. In some embodiments, the guide RNA includes an RNA sequence with at least 90%, at least 95%, at least 99% or 100% sequence identity to SEQ ID NO: 85. In some embodiments, the target site in the S7-RNase gene comprises a DNA sequence with at least 90%, at least 95%, at least 99% or 100% sequence identity to SEQ ID NO: 106.

In some embodiments, the method for the production of a self-fertile almond plants comprises providing a guided nuclease that is used to make a deletion in the S7-RNase gene, wherein the guided nuclease is directed to a target site by a guide RNA. In some embodiments, the guide RNA includes an RNA sequence with at least 90%, at least 95%, at least 99% or 100% sequence identity to SEQ ID NO: 85. In some embodiments, the target site in the S7-RNase gene comprises a DNA sequence with at least 90%, at least 95%, at least 99% or 100% sequence identity to SEQ ID NO: 106.

In some embodiments, the self-fertile almond plant includes an S7-RNase gene, wherein a portion of the S7-RNase gene has at least 90%, at least 95%, at least 99% or 100% sequence identity to SEQ ID NO: 6, except for a deletion (as compared to the respective, unedited S7-RNase gene) initiated by a guided nuclease in a region between about 20 bp upstream, and about 20 bp downstream (i.e., about +/−20 bp) from the 3′ end of a target site in the S7-RNase gene. In some embodiments, the guided nuclease has a guide RNA which includes an RNA sequence with at least 90%, at least 95%, at least 99% or 100% sequence identity to SEQ ID NO: 85. In some embodiments, the target site in the S7-RNase gene comprises a DNA sequence with at least 90%, at least 95%, at least 99% or 100% sequence identity to SEQ ID NO: 106.

In some embodiments, the method for the production of a self-fertile almond plant includes providing an S7-RNase gene, wherein a portion of the S7-RNase gene has at least 90%, at least 95%, at least 99% or 100% sequence identity to SEQ ID NO: 6, except for a deletion (as compared to the respective, unedited S7-RNase gene) initiated by a guided nuclease in a region between about 20 bp upstream, and about 20 bp downstream (i.e., about +/−20 bp) from the 3′ end of a target site in the S7-RNase gene. In some embodiments, the guided nuclease has a guide RNA which includes an RNA sequence with at least 90%, at least 95%, at least 99% or 100% sequence identity to SEQ ID NO: 85. In some embodiments, the target site in the S7-RNase gene comprises a DNA sequence with at least 90%, at least 95%, at least 99% or 100% sequence identity to SEQ ID NO: 106.

In some embodiments, a guided nuclease is used to make a deletion in the S7-SFB gene, wherein the guided nuclease is directed to a target site by a guide RNA. In some embodiments, the guide RNA includes an RNA sequence with at least 90%, at least 95%, at least 99% or 100% sequence identity to SEQ ID NO: 123. In some embodiments, the guide RNA includes an RNA sequence with at least 90%, at least 95%, at least 99% or 100% sequence identity to SEQ ID NO: 125. In some embodiments, the target site in the S7-SFB gene comprises a DNA sequence with at least 90%, at least 95%, at least 99% or 100% sequence identity to SEQ ID NO: 139. In some embodiments, the target site in the S7-SFB gene comprises a DNA sequence with at least 90%, at least 95%, at least 99% or 100% sequence identity to SEQ ID NO: 141.

In some embodiments, the method for the production of a self-fertile almond plant includes providing a guided nuclease that is used to make a deletion in the S7-SFB gene, wherein the guided nuclease is directed to a target site by a guide RNA. In some embodiments, the guide RNA includes an RNA sequence with at least 90%, at least 95%, at least 99% or 100% sequence identity to SEQ ID NO: 123. In some embodiments, the guide RNA includes an RNA sequence with at least 90%, at least 95%, at least 99% or 100% sequence identity to SEQ ID NO: 125. In some embodiments, the target site in the S7-SFB gene comprises a DNA sequence with at least 90%, at least 95%, at least 99% or 100% sequence identity to SEQ ID NO: 139. In some embodiments, the target site in the S7-SFB gene comprises a DNA sequence with at least 90%, at least 95%, at least 99% or 100% sequence identity to SEQ ID NO: 141.

In some embodiments, the self-fertile almond tree includes an S7-SFB gene, wherein a portion of the S7-SFB gene has at least 90%, at least 95%, at least 99% or 100% sequence identity to SEQ ID NO: 9, except for a deletion (as compared to the respective, unedited S7-SFB gene) initiated by a guided nuclease in a region between about 20 bp upstream, and about 20 bp downstream (i.e., about +/−20 bp) from the 3′ end of a target site in the S7-SFB gene. In some embodiments, the guided nuclease has a guide RNA which includes an RNA sequence with at least 90%, at least 95%, at least 99% or 100% sequence identity to SEQ ID NO: 123. In some embodiments, the guide RNA includes an RNA sequence with at least 90%, at least 95%, at least 99% or 100% sequence identity to SEQ ID NO: 125. In some embodiments, the target site in the S7-SFB gene comprises a DNA sequence with at least 90%, at least 95%, at least 99% or 100% sequence identity to SEQ ID NO: 139. In some embodiments, the target site in the S7-SFB gene comprises a DNA sequence with at least 90%, at least 95%, at least 99% or 100% sequence identity to SEQ ID NO: 141.

In some embodiments, the method for the production of a self-fertile almond tree includes providing an S7-SFB gene, wherein a portion of the S7-SFB gene has at least 90%, at least 95%, at least 99% or 100% sequence identity to SEQ ID NO: 9, except for a deletion (as compared to the respective, unedited S7-SFB gene) initiated by a guided nuclease in a region between about 20 bp upstream, and about 20 bp downstream (i.e., about +/−20 bp) from the 3′ end of a target site in the S7-SFB gene. In some embodiments, the guided nuclease has a guide RNA which includes an RNA sequence with at least 90%, at least 95%, at least 99% or 100% sequence identity to SEQ ID NO: 123. In some embodiments, the guide RNA includes an RNA sequence with at least 90%, at least 95%, at least 99% or 100% sequence identity to SEQ ID NO: 125. In some embodiments, the target site in the S7-SFB gene comprises a DNA sequence with at least 90%, at least 95%, at least 99% or 100% sequence identity to SEQ ID NO: 139. In some embodiments, the target site in the S7-SFB gene comprises a DNA sequence with at least 90%, at least 95%, at least 99% or 100% sequence identity to SEQ ID NO: 141.

### DETAILED DESCRIPTION

The following description is presented to enable a person of ordinary skill in the art to make and use the various embodiments disclosed herein. These embodiments are provided only as examples, as various modifications to the examples will be readily apparent to those of ordinary skill in the art.

In some embodiments, the present disclosure describes a self-fertile almond tree (Prunus dulcis) with one or more S-locus haplotypes, wherein one or more genes within the one or more S-locus haplotypes have a deletion as compared to the respective sequence of the unedited gene or genes. In some such embodiments, a deletion is between about 1 to 30 base pairs (“bp”). In some such embodiments, a deletion is between about 2 to 20 bp. In certain embodiments, the size of the deletion is not a multiple of 3. In certain embodiments, the one or more deletions substantially prevent expression of one or more S-locus gene products, resulting in a self-fertile almond tree.

The almond tree (Prunus dulcis) is a species of plant cultivated for its edible seed. The edible seed is otherwise referred to as an “almond.” Although almonds are a major agricultural tree crop, efficient almond production remains difficult due to a trait known as “self-incompatibility.”

In self-incompatible plants, such as Prunus dulcis for example, when a pollen grain contacts a stigma from the same plant, or a stigma from a different plant but with the same genotype (herein referred to as “self-pollen”), seed development does not occur. In other words, little to no almonds are produced by self-incompatible almond trees, unless one or more distinct varieties (having their own distinct genotypes) are grown in close proximity to provide a source of “non-self pollen.”

Current agronomic practice is therefore to stagger the planting of almond orchards with different varieties, such that enough genotypic diversity exists for pollinators (typically bees) to maximize flower fertilization and ultimately almond production. However, the problems with this current approach are numerous.

To begin with, not all almonds are equal. In fact, certain varieties like Prunus dulcis var. ‘Nonpareil’ and Prunus dulcis var. ‘Carmel’ produce almonds of such superior quality (for example, superior quality in performance categories such as taste, texture, processability, stability and the like) that these particular almonds command a higher price on the market. Due to this, a typical commercial almond orchard in, for example, the “Central Valley” Region of California, USA (the world's largest almond producing region), is primarily focused on the production of just a few high-quality almond varieties. In particular, the Nonpareil variety is considered the most desirable.

However, due to self-incompatibility, commercial orchards can only be planted with a high-quality almond variety like Nonpareil at about 30-50% maximum density per cultivated acre. The remaining 50-70% of their cultivated acreage must be interspersed with so-called “pollinator” varieties. Almonds harvested from pollinator varieties are typically of a lower quality, and therefore command a lower price on the market.

Furthermore, when multiple almond varieties are grown in close proximity, the differences in their individual biotic and abiotic requirements lead to further agronomic inefficiencies. For example, different spray applications are needed for certain varieties, depending on their particular susceptibilities to pests. Individual varieties may also have differences in their requirements for other factors such as amount and frequency of watering; the amount, composition and timing of fertilizer applications; differences in optimal soil compositions; differences in optimal harvest times; as well as differences in harvest methods, processing methods, and storage conditions.

All these inefficiencies are either directly or indirectly the result of self-incompatibility. The present disclosure provides methods for the production of a “self-fertile” high-quality almond variety (for example, a “self-fertile Nonpareil” or a “self-fertile Carmel”) that can produce almonds with self-pollen. Such trees could be planted at about 100% maximum density per cultivated acreage, eliminating the inefficiencies described above and maximizing the yield of high-quality almonds.

### S Haplotypes

Self-incompatibility is a mechanism which prevents self-fertilization by enabling the stigma to recognize and reject pollen from the same individual, as well as genetically related individuals (i.e., “self-pollen”). Self-incompatibility has been linked to a single multi-allelic locus called the “S locus” in Rosaceae (which includes Prunus dulcis) as well as in Solanaceae.

Studies have suggested that the S-locus in Prunus dulcis is highly variable, with at least 50 potential haplotypes in the known germplasm. See, e.g., S. N. Goonetilleke et al., Variation among S-locus haplotypes and among stylar RNases in almond. Sci Rep 10, 583 (2020) (incorporated herein by reference with respect to its disclosure regarding the S-locus). Among the haplotypes that have been physically mapped, the order and orientations of genetic features are conserved, but the percentage of sequence identity and distances between the features vary between haplotype.

Each known S haplotype contains, among other features, two genes which are believed to affect self-incompatibility-one being stigma-related, and the other one being pollen-related. The stigma-related gene encodes a glycoprotein with ribonuclease activity known as “S-RNase.” The pollen-related gene encodes a F-box protein with E3 ubiquitin ligase activity known as “SFB.”

The so-called “S8 haplotype” is found in some of the highest quality, most-desirable almond varieties such as ‘Nonpareil’ and ‘Carmel’. ‘Nonpareil’ contains the S8 haplotype as well as the “S7 haplotype” (2n=S8/S7), whereas ‘Carmel’ contains the S8 haplotype as well as the “S5 haplotype” (2n=S8/S5).

### S-RNase

S-RNase is soluble glycoprotein with RNase activity. This protein is believed to enable the stigma to recognize and arrest fertilization of self-pollen. The allelic variants of S-RNase share several conserved regions but also contain one hypervariable region.

The transcribed portion of the S8-RNase gene (including both exons and introns, i.e., the “pre-mRNA sequence”) is provided by SEQ ID NO: 1. The protein coding sequence for S8-RNase (i.e., exons only) is provided by SEQ ID NO: 2. The amino acid sequence of the S8-RNase protein is provided by SEQ ID NO: 3.

The transcribed portion of the S7-RNase gene (including both exons and introns, i.e., the “pre-mRNA sequence”) is provided by SEQ ID NO: 6. The protein coding sequence for S7-RNase (i.e., exons only) is provided by SEQ ID NO: 7. The amino acid sequence of the S7-RNase protein is provided by SEQ ID NO: 8.

These sequences were obtained from the unedited ‘Nonpareil’ variety, as described in the Examples section below.

### SFB Protein

The SFB protein is a component of a class of E3 ubiquitin ligases. It is believed to function as a receptor to incorporate proteins targeted for polyubiquitination and degradation by the 26S proteasome. In Prunus, SFB contains a single intron which varies in length depending on the haplotype. Furthermore, SFB in Prunus has two variable regions (referred to as “V1” and “V2”) and two hypervariable regions (referred to as “HVa” and “HVb”).

The transcribed portion of the S8-SFB gene is provided by SEQ ID NO: 4. There are no introns present in the S8-SFB gene, therefore the transcribed sequence is the same sequence as the protein coding sequence. The amino acid sequence of the S8-SFB protein is provided by SEQ ID NO: 5.

The transcribed portion of the S7-SFB gene is provided by SEQ ID NO: 9. There are no introns present in the S7-SFB gene, therefore the transcribed sequence is the same sequence as the protein coding sequence. The amino acid sequence of the S7-SFB protein is provided by SEQ ID NO: 10.

These sequences were obtained from the unedited ‘Nonpareil’ variety, as described in the Examples section below.

### Genetic Deletions

Any method that is effective at making deletions in a nucleotide sequence can be used in conjunction with the teachings herein. Deletions can be achieved through targeted damage to the DNA strand at a target site in the allele or alleles of interest. Cellular repair of the targeted damage (in the absence of an externally provided repair template) results in the deletion. There are several known gene editing techniques which theoretically may be used to introduce such deletions, and which rely on natural or engineered nucleases. These methods are known in the art. They include but are not limited to the use of RNA-guided nucleases (hereinafter referred to as “guided nucleases”) and site-specific nucleases such as meganucleases, zinc finger nucleases (ZFNs), and transcription activator-like effector-based nucleases (TALENs) (hereinafter referred to together as “site-specific nucleases”).

In some embodiments, the self-fertile almond trees disclosed herein include one or more deletions in one or more genes from one or more S-locus haplotypes.

In some embodiments, the self-fertile almond tree has a deletion in S8-RNase.

In some embodiments, the self-fertile almond tree has a deletion in S8-SFB.

In some embodiments, the self-fertile almond tree has a deletion in S7-RNase.

In some embodiments, the self-fertile almond tree has a deletion in S7-SFB.

In some embodiments, the self-fertile almond tree has a deletion in S5-RNase.

In some embodiments, the self-fertile almond tree has a deletion in S5-SFB.

In certain embodiments, the length of the deletion is not a multiple of three, resulting in a frameshift mutation. In some embodiments, the deletion is 1 bp, 2 bp, 4 bp, 5 bp, 7 bp, 8 bp, 10 bp, 11 bp, 13 bp, 14 bp, 16 bp, 17 bp, 19 bp, 20 bp, 22 bp, 23 bp, 25 bp, 26 bp, 28 bp, 29, 31 bp, 32 bp, 34 bp or 35 bp in length. In some embodiments, the one or more deletions substantially prevent expression of one or more S-locus gene products, resulting in a self-fertile almond tree.

In certain embodiments, the length of the deletion is not a multiple of three, resulting in an in-frame mutation. In some embodiments, the deletion is 1 bp, 2 bp, 4 bp, 5 bp, 7 bp, 8 bp, 10 bp, 11 bp, 13 bp, 14 bp, 16 bp, 17 bp, 19 bp, 20 bp, 22 bp, 23 bp, 25 bp, 26 bp, 28 bp, 29, 31 bp, 32 bp, 34 bp or 35 bp in length. In some embodiments, the one or more deletions substantially prevent expression of one or more S-locus gene products, resulting in a self-fertile almond tree.

The deletions may occur within any genetic elements within the one or more genes from the one or more S-locus haplotypes. The genetic elements include, but are not limited to, protein-coding sequences, non-protein-coding sequences, promoter regions, 5′ untranslated leaders, genes, exons, introns, poly-A signal sequences, 3′ untranslated regions, regions encoding small RNAs (such as microRNAs and small-interfering RNAs), and any other sequences that affect transcription or translation of the one or more genes.

In some embodiments, the deletions cause gene knockout, gene knockdown, or a combination thereof. As used herein, a “gene knockout” is a genetic modification wherein a gene or gene product has been rendered completely inoperative. As used herein, a “gene knockdown” is a genetic modification wherein a gene or gene product has been rendered partially inoperative.

### Guided Nucleases

A guided nuclease functions within a ribonucleoprotein (RNP) complex to cleave a DNA strand at a target site. The target site is determined by a so-called “guide nucleic acid.” In some embodiments, the guide nucleic acid is a “guide RNA.”

According to some embodiments, the guided nuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, CasZ, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions thereof). According to some particular embodiments, the guided nuclease is a Cpf1 nuclease.

In some embodiments, the guide nucleic acid is provided to direct the guided nuclease to a specific “target site” in the DNA. This occurs via base-pairing or hybridization between a portion of the guide nucleic acid and the target DNA, thereby allowing the nuclease to induce a double-stranded DNA (“dsDNA”) break or single-stranded DNA (“ssDNA”) nick at or near the target site. The “target site” sequence can be on either the sense (+) strand or the antisense (−) strand, despite that only the sense (+) strand sequence is explicitly provided herein. As one of ordinary skill would appreciate, the antisense (−) strand sequence is inherently described by the sense (+) strand sequence because the antisense (−) strand sequence is the reverse complement of the sense (+) strand sequence. For example, SEQ ID NO: 76 provides a target site in the antisense strand of S8-SFB. In order to locate this target site in the corresponding sense (+) strand sequence (i.e., SEQ ID NO: 4, the transcribed portion of the S8-SFB gene) one of ordinary skill would search the reverse complement sequence. In certain embodiments, the guide RNA is introduced into a plant cell or plant tissue as an RNA molecule. In other embodiments, the guide RNA is introduced as a recombinant DNA molecule, construct or vector comprising a transcribable DNA sequence encoding the guide RNA. In the latter embodiment, the transcribable DNA sequence encoding the guide RNA is operably linked to a plant-expressible promoter. The promoter may be any type of promoter including a constitutive promoter, a tissue-specific promoter or tissue-preferred promoter, a developmental stage promoter, or an inducible promoter.

In some embodiments, the guide nucleic acid includes a first segment with a nucleotide sequence that is complementary to a sequence in the target DNA and a second segment that interacts with the guided nuclease. In some embodiments, the first segment of the guide nucleic acid includes a nucleotide sequence that is complementary to a sequence in the target DNA that corresponds to a CRISPR RNA repeat (hereinafter “crRNA” or “crRNA repeat”). In some embodiments, the guide nucleic acid comprises two separate nucleic acid molecules (a polynucleotide that is complementary to a sequence in a target nucleic acid and a polynucleotide that interacts with a guided nuclease protein) that hybridize with one another. In some embodiments, the guide nucleic acid may include RNA, DNA, a combination of RNA and DNA, or other nucleic acid modifications known in the art to improve guide efficiency.

A protospacer-adjacent motif (“PAM”) is present in the genome immediately adjacent and upstream to the 5′ end of the target genomic site sequence, and complementary to the targeting sequence of the guide RNA immediately downstream (3′) to the sense (+) strand of the genomic target site (relative to the targeting sequence of the guide RNA). Such considerations have been characterized and are known in the art.

The genomic PAM sequence on the sense (+) strand adjacent to the target site (relative to the targeting sequence of the guide RNA) may comprise 5′-NGG-3′. However, the corresponding sequence of the guide nucleic acid (immediately downstream (3′) to the targeting sequence of the guide RNA) may generally not be complementary to the genomic PAM sequence. See, e.g., Wu, X. et al., Target specificity of the CRISPR-Cas9 system, Quant Biol. 2 (2): 59-70 (2014) (incorporated by reference herein regarding its disclosure on guide strand design and PAM sites).

The guide nucleic acid is typically a non-coding RNA molecule that does not encode a protein. The targeting sequence of the guide nucleic acid may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The targeting sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the target site.

In addition to the targeting sequence, a guide nucleic acid may further comprise one or more other structural or scaffold sequence(s), which may bind or interact with a guided nuclease. Such scaffold or structural sequences may further interact with other RNA molecules (e.g., trans-acting crRNA).

In some embodiments, a guided nuclease is used to make a deletion in the S8-RNase gene, wherein the guided nuclease is directed to a target site by a guide RNA. In some embodiments, the guide RNA includes an RNA sequence with at least 90%, at least 95%, at least 99% or 100% sequence identity to SEQ ID NO: 17. In some embodiments, the guide RNA includes an RNA sequence with at least 90%, at least 95%, at least 99% or 100% sequence identity to SEQ ID NO: 18. In some embodiments, the target site in the S8-RNase gene comprises a DNA sequence with at least 90%, at least 95%, at least 99% or 100% sequence identity to SEQ ID NO: 37. In some embodiments, the target site in the S8-RNase gene comprises a DNA sequence with at least 90%, at least 95%, at least 99% or 100% sequence identity to SEQ ID NO: 38.

In some embodiments, the self-fertile almond tree includes an S8-RNase gene, wherein a portion of the S8-RNase gene has at least 90%, at least 95%, at least 99% or 100% sequence identity to SEQ ID NO: 1, except for a deletion (as compared to the respective, unedited S8-RNase gene) initiated by a guided nuclease in a region between about 20 bp upstream, and about 20 bp downstream (i.e., about +/−20 bp) from the 3′ end of a target site in the S8-RNase gene. In some embodiments, the guided nuclease has a guide RNA which includes an RNA sequence with at least 90%, at least 95%, at least 99% or 100% sequence identity to SEQ ID NO: 17. In some embodiments, the guide RNA includes an RNA sequence with at least 90%, at least 95%, at least 99% or 100% sequence identity to SEQ ID NO: 18. In some embodiments, the target site in the S8-RNase gene comprises a DNA sequence with at least 90%, at least 95%, at least 99% or 100% sequence identity to SEQ ID NO: 37. In some embodiments, the target site in the S8-RNase gene comprises a DNA sequence with at least 90%, at least 95%, at least 99% or 100% sequence identity to SEQ ID NO: 38.

In some embodiments, a guided nuclease is used to make a deletion in the S8-SFB gene, wherein the guided nuclease is directed to a target site by a guide RNA. In some embodiments, the guide RNA includes an RNA sequence with at least 90%, at least 95%, at least 99% or 100% sequence identity to SEQ ID NO: 57. In some embodiments, the target site in the S8-SFB gene comprises a DNA sequence with at least 90%, at least 95%, at least 99% or 100% sequence identity to SEQ ID NO: 70.

In some embodiments, the self-fertile almond tree includes an S8-SFB gene, wherein a portion of the S8-SFB gene has at least 90%, at least 95%, at least 99% or 100% sequence identity to SEQ ID NO: 4, except for a deletion (as compared to the respective, unedited S8-SFB gene) initiated by a guided nuclease in a region between about 20 bp upstream, and about 20 bp downstream (i.e., about +/−20 bp) from the 3′ end of a target site in the S8-SFB gene. In some embodiments, the guided nuclease has a guide RNA which includes an RNA sequence with at least 90%, at least 95%, at least 99% or 100% sequence identity to SEQ ID NO: 57. In some embodiments, the target site in the S8-SFB gene comprises a DNA sequence with at least 90%, at least 95%, at least 99% or 100% sequence identity to SEQ ID NO: 70.

In some embodiments, a guided nuclease is used to make a deletion in the S7-RNase gene, wherein the guided nuclease is directed to a target site by a guide RNA. In some embodiments, the guide RNA includes an RNA sequence with at least 90%, at least 95%, at least 99% or 100% sequence identity to SEQ ID NO: 85. In some embodiments, the target site in the S7-RNase gene comprises a DNA sequence with at least 90%, at least 95%, at least 99% or 100% sequence identity to SEQ ID NO: 106.

In some embodiments, the self-fertile almond tree includes an S7-RNase gene, wherein a portion of the S7-RNase gene has at least 90%, at least 95%, at least 99% or 100% sequence identity to SEQ ID NO: 6, except for a deletion (as compared to the respective, unedited S7-RNase gene) initiated by a guided nuclease in a region between about 20 bp upstream, and about 20 bp downstream (i.e., about +/−20 bp) from the 3′ end of a target site in the S7-RNase gene. In some embodiments, the guided nuclease has a guide RNA which includes an RNA sequence with at least 90%, at least 95%, at least 99% or 100% sequence identity to SEQ ID NO: 85. In some embodiments, the target site in the S7-RNase gene comprises a DNA sequence with at least 90%, at least 95%, at least 99% or 100% sequence identity to SEQ ID NO: 106.

In some embodiments, a guided nuclease is used to make a deletion in the S7-SFB gene, wherein the guided nuclease is directed to a target site by a guide RNA. In some embodiments, the guide RNA includes an RNA sequence with at least 90%, at least 95%, at least 99% or 100% sequence identity to SEQ ID NO: 123. In some embodiments, the guide RNA includes an RNA sequence with at least 90%, at least 95%, at least 99% or 100% sequence identity to SEQ ID NO: 125. In some embodiments, the target site in the S7-SFB gene comprises a DNA sequence with at least 90%, at least 95%, at least 99% or 100% sequence identity to SEQ ID NO: 139. In some embodiments, the target site in the S7-SFB gene comprises a DNA sequence with at least 90%, at least 95%, at least 99% or 100% sequence identity to SEQ ID NO: 141.

In some embodiments, the self-fertile almond tree includes an S7-SFB gene, wherein a portion of the S7-SFB gene has at least 90%, at least 95%, at least 99% or 100% sequence identity to SEQ ID NO: 9, except for a deletion (as compared to the respective, unedited S7-SFB gene) initiated by a guided nuclease in a region between about 20 bp upstream, and about 20 bp downstream (i.e., about +/−20 bp) from the 3′ end of a target site in the S7-SFB gene. In some embodiments, the guided nuclease has a guide RNA which includes an RNA sequence with at least 90%, at least 95%, at least 99% or 100% sequence identity to SEQ ID NO: 123. In some embodiments, the guide RNA includes an RNA sequence with at least 90%, at least 95%, at least 99% or 100% sequence identity to SEQ ID NO: 125. In some embodiments, the target site in the S7-SFB gene comprises a DNA sequence with at least 90%, at least 95%, at least 99% or 100% sequence identity to SEQ ID NO: 139. In some embodiments, the target site in the S7-SFB gene comprises a DNA sequence with at least 90%, at least 95%, at least 99% or 100% sequence identity to SEQ ID NO: 141.

In some embodiments, one or more guided nucleases are used to make one or more combinations of the deletions described above.

### Site-Specific Nucleases

The nuclease may be a site-specific nuclease. Several site-specific nucleases, such as recombinases, zinc finger nucleases (ZFNs), meganucleases, and TALENs, are not guided by a nucleic acid, but instead rely on their own protein structure to determine their target site. In additional embodiments, a site-specific nuclease may be fused, tethered or otherwise attached to a DNA-binding protein domain or motif. The protein structure of the site-specific nuclease (or the fused/attached/tethered DNA binding domain) may target the site-specific nuclease to the target site. According to many of these embodiments, site-specific nucleases such as recombinases, zinc finger nucleases (ZFNs), meganucleases, and TALENs, may theoretically be designed, engineered and constructed according to known methods to target and bind to a target site in a DNA. The site-specific nuclease then induces targeted DNA damage (e.g., a DSB or nick) occurs similarly leading to the creation of a mutation or insertion of a sequence at the site of the DSB or nick, through cellular repair mechanisms, which may be guided by a donor template molecule.

In some embodiments, a site-specific nuclease is a recombinase. A recombinase may be a serine recombinase attached to a DNA recognition motif, a tyrosine recombinase attached to a DNA recognition motif, or other recombinase enzyme known in the art. A recombinase or transposase may be a DNA transposase or recombinase attached or fused to a DNA binding domain. Non-limiting examples of recombinases include a tyrosine recombinase attached, etc., to a DNA recognition motif provided herein is selected from the group consisting of a Cre recombinase, a Gin recombinase, a Flp recombinase, and a Tnp1 recombinase. In an aspect, a Cre recombinase or a Gin recombinase provided herein is tethered to a zinc-finger DNA-binding domain, or a TALE DNA-binding domain, or a guided nuclease (e.g., Cas9 or Cpf1). In another aspect, a serine recombinase attached to a DNA recognition motif provided herein is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

A site-specific nuclease may be a zinc finger nuclease (ZFN). ZFNs are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger .alpha.-helix, which contribute to site-specific binding to the target site, can be changed and customized to fit specific target sequences. The other amino acids may form a consensus backbone to generate ZFNs with different sequence specificities.

Methods and rules for designing ZFNs for targeting and binding to specific target sequences are known in the art. See, e.g., U.S. patent application Nos. 2005/0064474, 2009/0117617, and 2012/0142062 (herein incorporated by reference regarding its disclosure of ZFN design). The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site. Without being limited by any theory, because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZFN provided herein is capable of generating a targeted DSB or nick.

A site-specific nuclease may be a TALEN enzyme. TALENs are artificial restriction enzymes generated by fusing the transcription activator-like effector (TALE) DNA binding domain to a nuclease domain (e.g., FokI). When each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

TALENs are artificial restriction enzymes generated by fusing the transcription activator-like effector (TALE) DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Transcription activator-like effectors (TALEs) can be engineered to bind practically any DNA sequence, such as at or near the genomic locus of a gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al. 2013. PLoS One. 8: e82539). MutH is capable of introducing strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4:1762).

The relationship between amino acid sequence and DNA recognition of the TALE binding domain allows for designable proteins. Software programs such as DNAWorks can be used to design TALE constructs. Other methods of designing TALE constructs are known to those of skill in the art. See Doyle et al., Nucleic Acids Research (2012) 40: W117-122; Cermak et al., Nucleic Acids Research (2011) 39: e82; and tale-nt.cac.cornelledu/about. In another aspect, a TALEN provided herein is capable of generating a targeted DSB.

A site-specific nuclease may be a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-CeuI, I-MsoI, I-SceI, I-AniI, and I-DmoI. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target sequence in a plant, such as at or near the genomic locus of a gene. In another aspect, a meganuclease provided herein is capable of generating a targeted DSB.

In some embodiments, the introduction of the proteins, nucleic acids, or combination thereof into the plant cell comprises isolating protoplasts, transfecting the protoplasts, encapsulating the protoplasts, and regenerating plants from the protoplasts. In other embodiments, the introduction of the proteins, nucleic acids, or combination thereof into the plant cell comprises biolistic transformation. In certain embodiments, the introduction of the proteins, nucleic acids, or combination thereof into the plant cell comprises isolating immature plant embryos, bombarding the embryos with particles comprising nucleic acids, and regenerating plants from the immature embryos.

Additional transformation methods may be used to introduce the proteins, nucleic acids, or combination thereof into a suitable plant or plant cell. Transformation methods include the use of liposomes, electroporation, chemicals that increase free DNA uptake, injection of the DNA directly into the plant (cell) such as microinjection, particle gun bombardment, transformation using viruses or pollen and microprojection. Methods may be selected from the calcium/polyethylene glycol method for protoplasts (Krens et al. (1982) Nature 296:72-74; Negrutiu et al. (1987) Plant. Mol. Biol. 8:363-373); electroporation of protoplasts (Shillito et al. (1985) Bio/Technol. 3:1099-1102); microinjection into plant material (Crossway et al. (1986) Mol. Gen. Genet. 202:179-185); DNA or RNA-coated particle bombardment (Klein et al. (1987) Nature 327:70) infection with (non-integrative) viruses and the like.

### Plants, Plant Parts, Plant Cells, and Processed Plant Products

In another aspect, provided herein are self-fertile almond plants, as well as plant parts, plant tissues and/or plant cells derived therefrom. Also provided herein are processed plant products derived from the self-fertile almond plants, plant parts, or plant cells provided herein.

In some embodiments, self-fertile almond plants and plant parts are provided herein. The self-fertile almond plants and plant parts may be grown from a self-fertile almond tree (Prunus dulcis) with one or more S-locus haplotypes, wherein one or more genes within the one or more S-locus haplotypes have a deletion as compared to the respective sequence of the unedited gene or genes, as described herein.

“Plant parts” can be obtained by cutting, snapping, grinding or otherwise disassociating the part from the plant. A plant part may be any plant part known in the art, including, but not limited to, a flower, a pistil, a leaf, a stem, a petiole, a cutting, a tissue, a seed coat, an ovule, pollen, a root, a rootstock, a scion, a fruit, a cotyledon, a hypocotyl, a protoplast, an embryo, an anther, a seed, a boll, or any portion thereof. In certain embodiments, the plant part is the seed of a self-fertile almond tree.

In some embodiments, provided herein is a “processed plant product” derived from a self-fertile almond plant, plant part, or plant cell described herein. A “processed plant product” may be a seed, a fruit, a grain, a root, a vegetable, or any plant part described herein, and may be blended as a commodity or other product which moves through commerce and is derived from a genetically modified plant or a genetically modified plant part.

In some embodiments, the commodity or other product can be tracked through commerce by detecting nucleic acid and/or protein sequences of the self-fertile almond trees described herein. In certain embodiments, the processed plant product comprises a detectable amount of nucleotide and/or protein sequences corresponding to the one or more S-locus haplotypes described herein, wherein one or more genes within the one or more S-locus haplotypes have a deletion as described herein.

In some embodiments, the commodity or other product is produced in or maintained in the self fertile almond plant, or plant part, from which the commodity or other product has been obtained. Such commodities or other products of commerce include, but are not limited to, plant parts, biomass, oil, meal, food starch, syrup, sugar, animal feed, flour, flakes, bran, lint, processed seed, and seed. The processed plant product may be a food product that is processed by any means known in the art, e.g., canned, steamed, boiled, fried, blanched and/or frozen etc.

Also provided herein is a cell culture or tissue culture of regenerable cells or tissue of the self-fertile almond tree described herein. The regenerable cells are derived from embryos, protoplasts, meristematic cells, callus, pollen, leaves, anthers, stems, petioles, roots, root tips, fruits, seeds, flowers, cotyledons, and/or hypocotyls of a self-fertile almond tree as described herein.

### EXAMPLES

The presently disclosed subject matter will be better understood by reference to the following Examples, which are provided as exemplary of the inventions, and not by way of limitation.

### Example 1: General Methods

### Example 1A: Sequencing and Annotating the S8-Locus and S7-Locus

DNA was extracted from the leaves of young in vitro plantlets. About 1 to 10 g of leaf tissue was flash-frozen in liquid Nitrogen and ground using a mortar and pestle. Homogenate was suspended in an osmotically balanced buffer and filtered through miracloth. The filtered solution was centrifuged and washed several times in a mild detergent solution. DNA was isolated from cells using a cetyltrimethylammonium bromide (“CTAB”) solution and purified using chloroform. PacBio HiFi sequencing was performed at the UC Davis Genome Center on a Sequel II instrument.

HiFi reads were assembled with the Hifiasm assembler (v0.16.1-r375) (as described, for example, in Cheng et al. Haplotype-resolved de novo assembly using phased assembly graphs with hifiasm. Nat Methods 18, 170-175 (2021)). Contigs were screened to remove organellar and contaminant contigs using previously described methods (see, e.g., Alonge et al., Automated assembly scaffolding using RagTag elevates a new tomato system for high-throughput genome editing. Genome Biol 23, 258 (2022)). Screened contigs were scaffolded with RagTag (v2.1.0, —aligner nucmer—nucmer-params “—maxmatch-1 100-c 500”) (see, e.g., Alonge et al., 2022) using a previously published ‘Nonpareil’ genome assembly as the reference from D'Amico-Willman et al., Whole-genome sequence and methylome profiling of the almond [Prunus dulcis (Mill.) D. A. Webb] cultivar ‘Nonpareil’. G3 (Bethesda). 2022 May 6; 12(5):jkac065. doi: 10.1093/g3journal/jkac065. Erratum in: G3 (Bethesda). 2022 May 30; 12(6); PMID: 35325123; PMCID: PMC9073694). Mitochondrial (CM038036.2) and plastid (CM038037.1) genome sequences were added to the final assembly.

Liftoff (v1.6.3, -copies -cds) was used to annotate the new assembly given annotations from the previously published ‘Nonpareil’ reference genome annotation (See Shumate & Salzberg, Liftoff: accurate mapping of gene annotations, Bioinformatics, Volume 37, Issue 12, 15 Jun. 2021, Pages 1639-1643). Initial annotations were manually curated at loci of interest.

### Example 1B: Identification of Target DNA Sequences and crRNA Design

S8-RNase (SEQ ID NO: 1) and S8-SFB (SEQ ID NO: 4) within the S8-locus, and S7-RNase (SEQ ID NO: 6) and S7-SFB (SEQ ID NO: 9) within the S7-locus, were targeted for crRNA design in Geneious Prime 2020.0.3 using PAM sites for an appropriate cas nuclease. crRNA with high specificity targeting the first or second exons or promoter sequences were used for testing, but high-scoring crRNA targeting later exons were also selected.

The target DNA sequences for S8-RNase are provided by SEQ ID NOs: 31-50. The target DNA sequences for S8-SFB are provided by SEQ ID NOs: 64-76. The target DNA sequences for S7-RNase provided by SEQ ID NOs: 98-118. The target DNA sequences for S7-SFB provided by SEQ ID NOs: 135-150.

Resulting crRNA sequences were exported and synthesized by IDT using standard crRNA synthesis. The crRNA sequences for S8-RNase are provided by SEQ ID NOs: 11-30. The crRNA sequences for S8-SFB are provided by SEQ ID NOs: 51-63. The crRNA sequences for S7-RNase are provided by SEQ ID NOs: 77-97. The crRNA sequences for S7-SFB are provided by SEQ ID NOs: 119-134.

### Example 1C: Ribonucleoprotein Preparation

To prepare ribonucleoproteins (RNPs), 2 μL of NEB buffer 2.1 (10× stock) was placed into a 1.5 mL microcentrifuge tube with 10-40 μg of crRNA with an equal mass of the cas nuclease protein, and the final volume is adjusted to 20 μL using nuclease free water. The solution is prepared fresh and used after a 15 minute incubation at room temperature.

### Example 1D: Confirmation of Targeted Deletions

To confirm the presence of targeted deletions, primers were first designed to amplify each gene corresponding to the reference genome. One or more long range direct PCRs were then performed using crude lysate from transfected protoplasts after 24 to 48 hours incubation at room temperature. PCR products were pooled by transfection sample and a Seqwell library preparation was performed to generate an Illumina library. In sum, PCR amplicons were used to prepare the Illumina sequencing libraries using plexWell 96 kits (SeqWell, Beverly, Massachusetts) and libraries were sequenced on an Illumina iSeq (Illumina, San Diego, California) with a Paired End 150 nt sequencing kit.

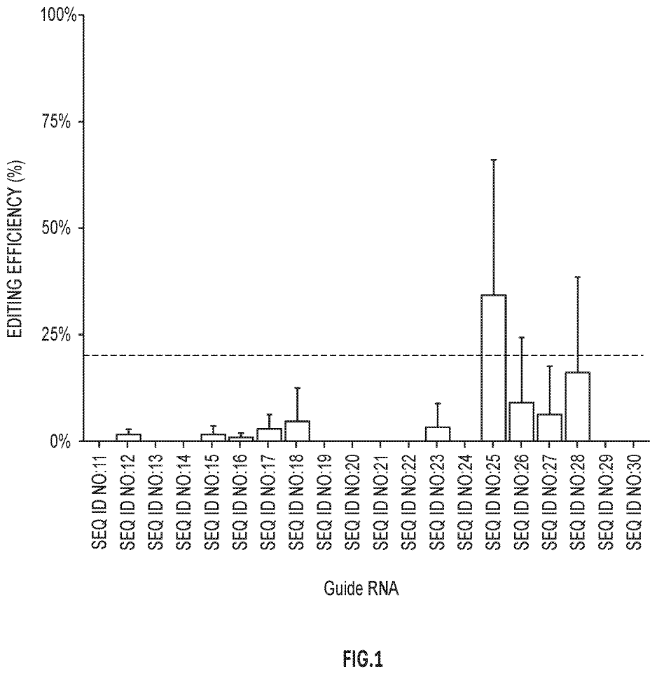

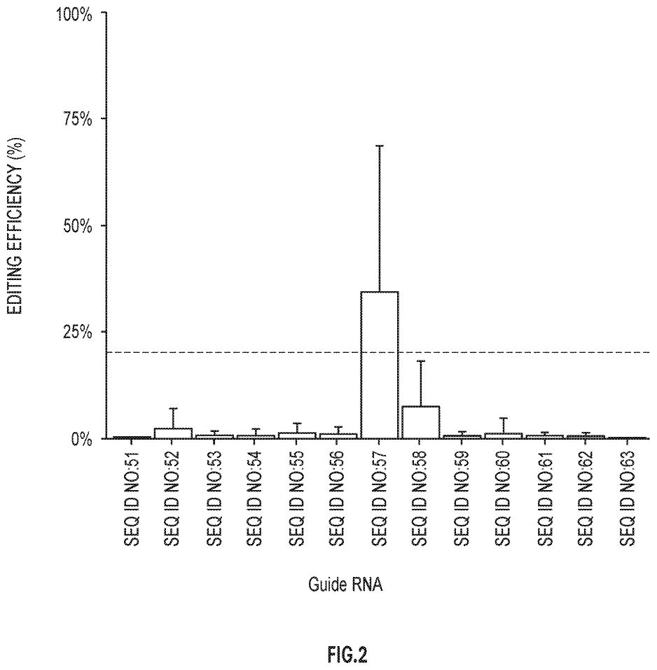

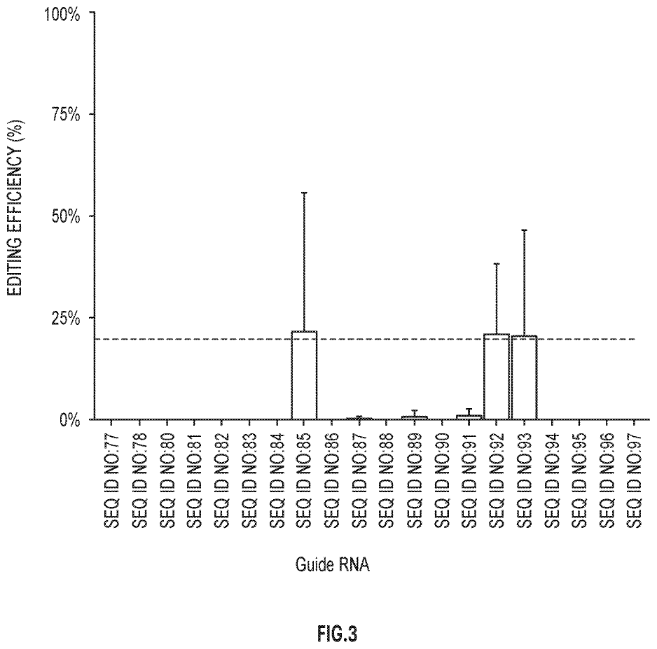

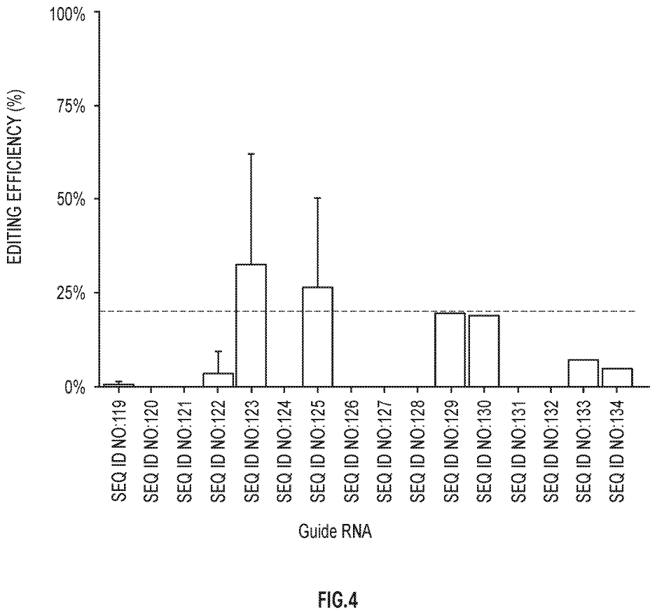

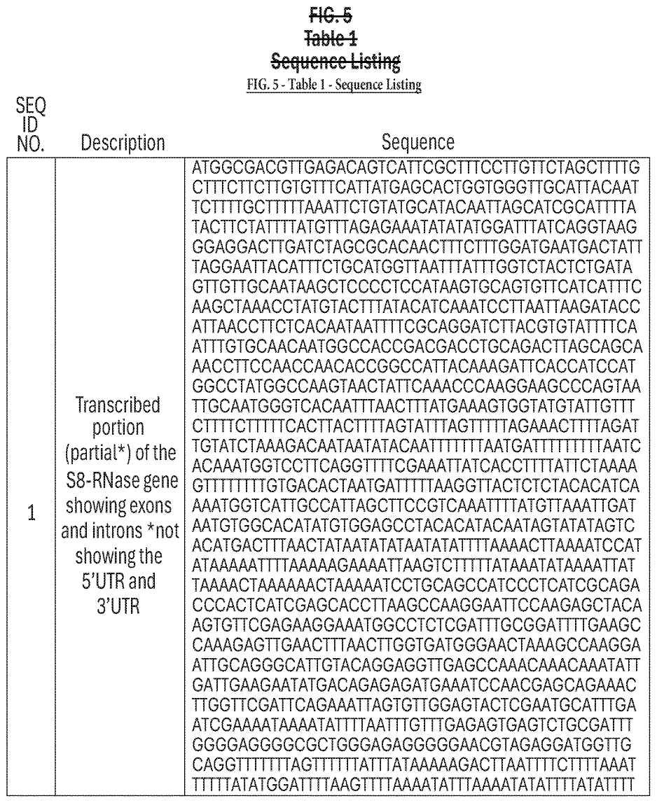

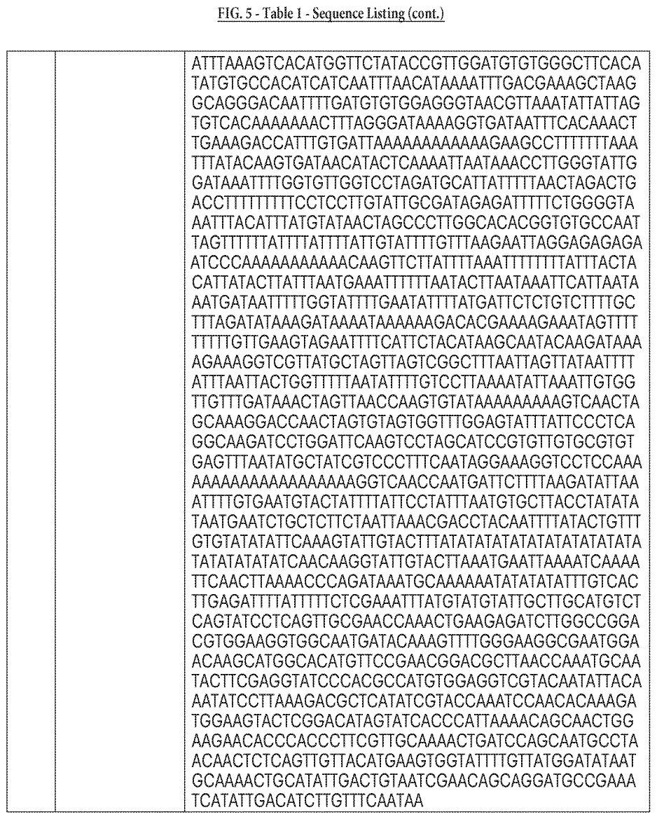

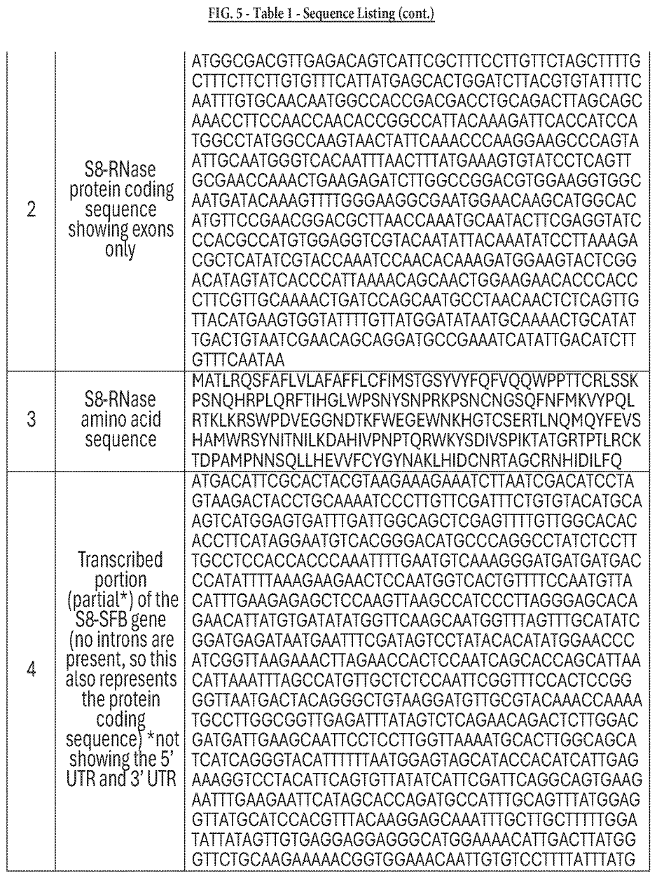

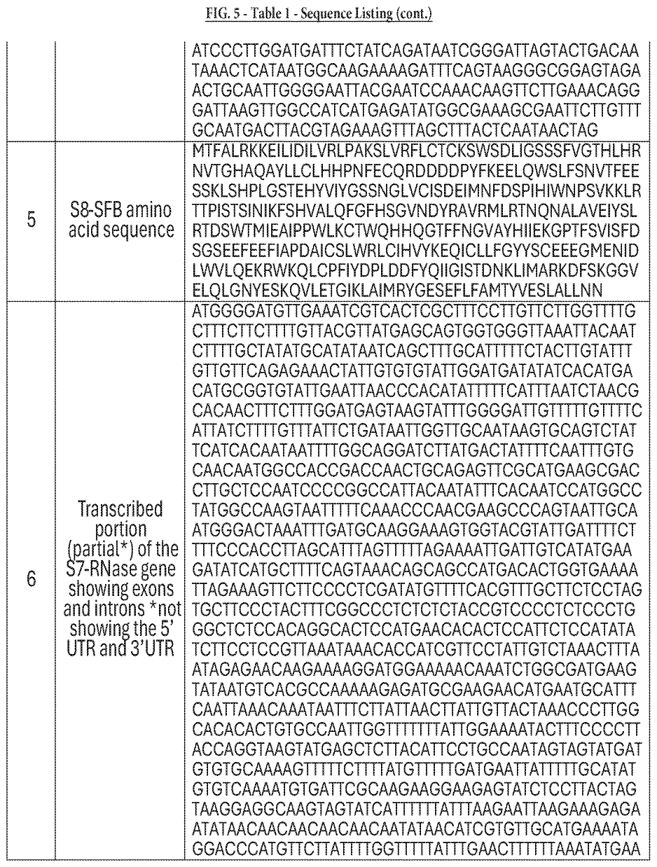

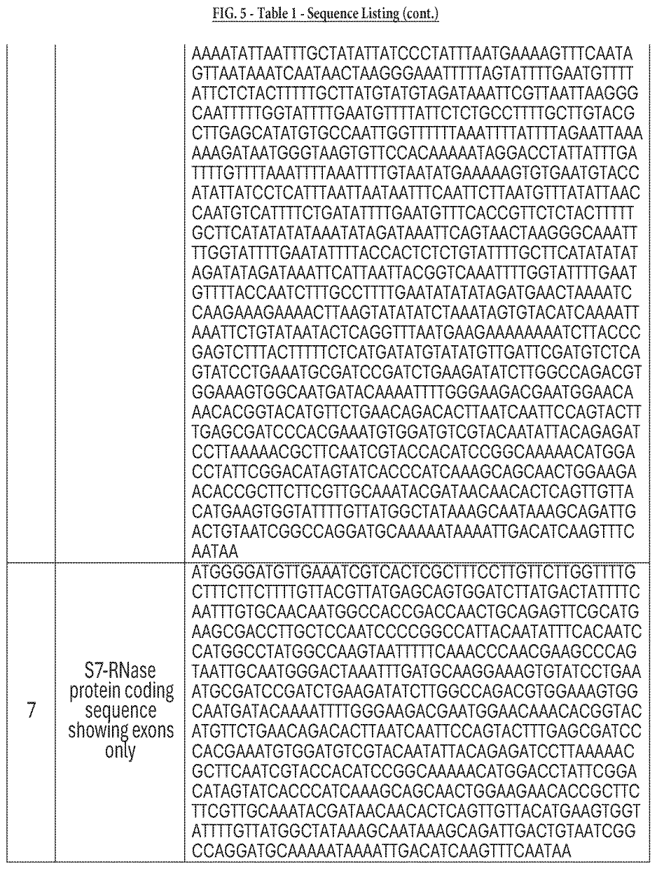

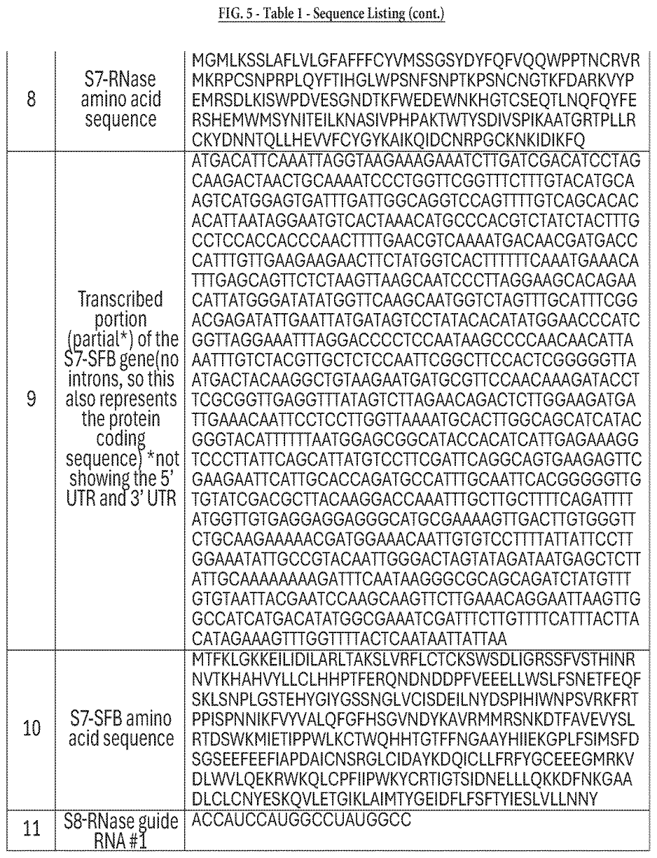

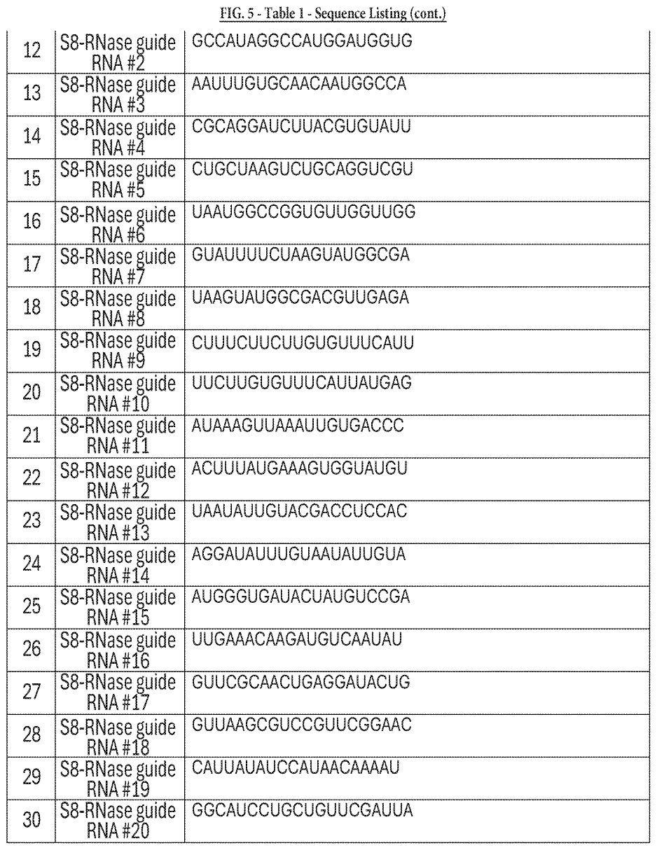

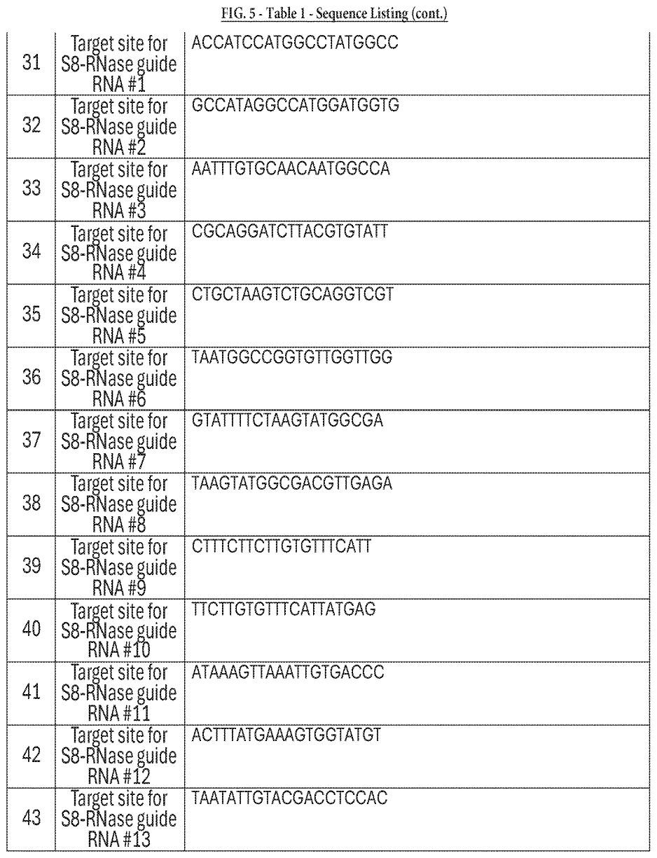

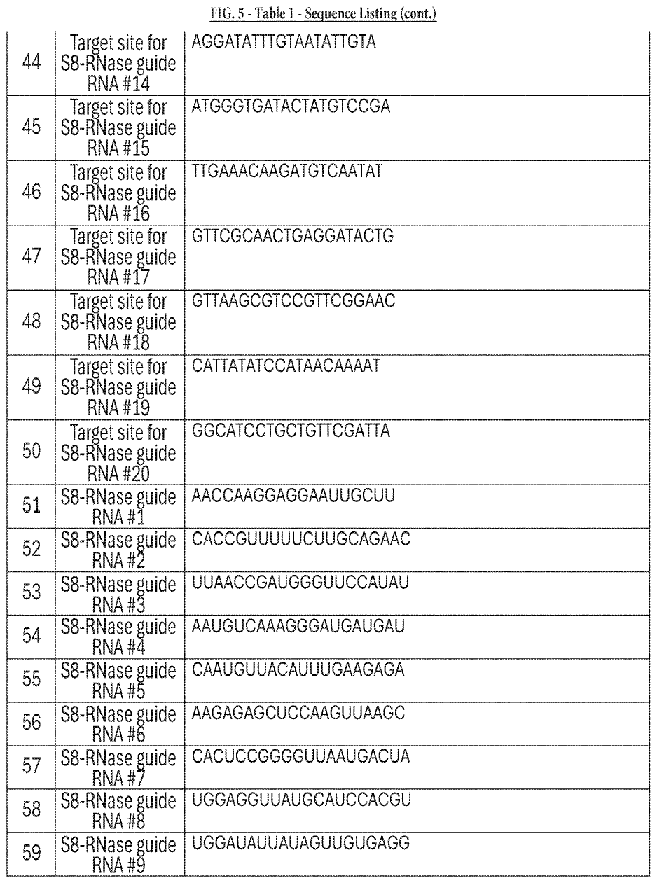

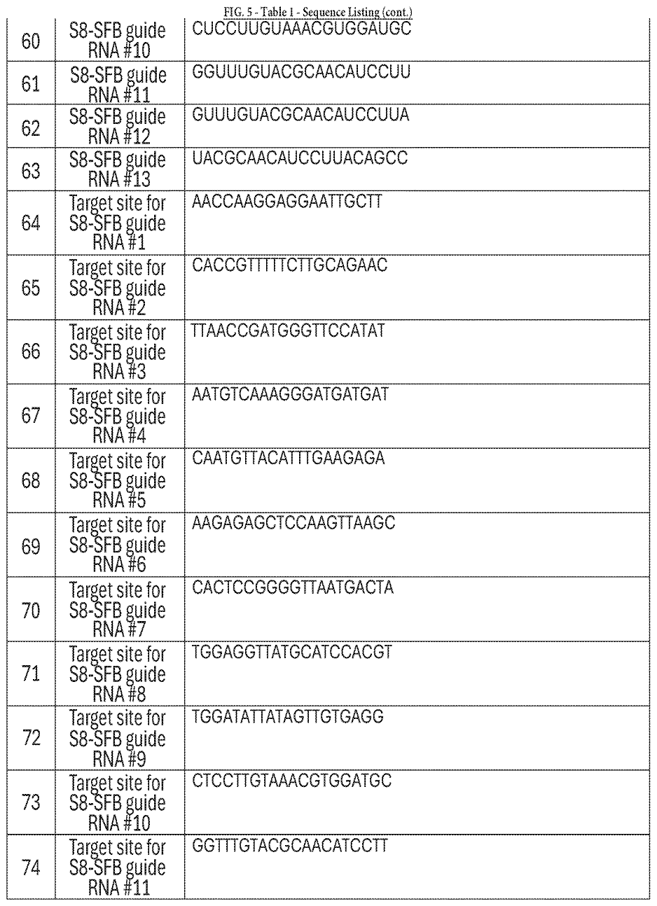

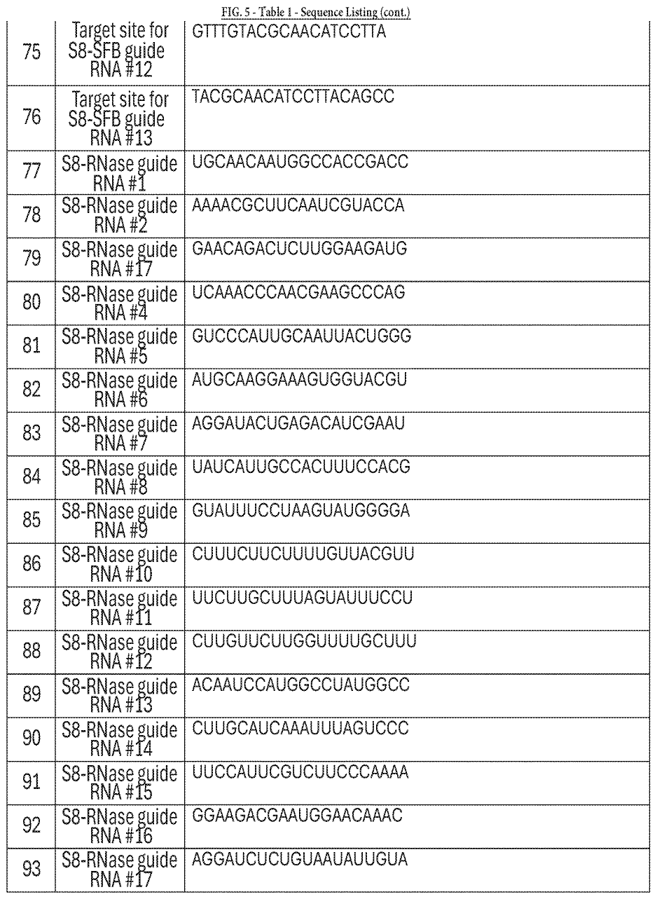

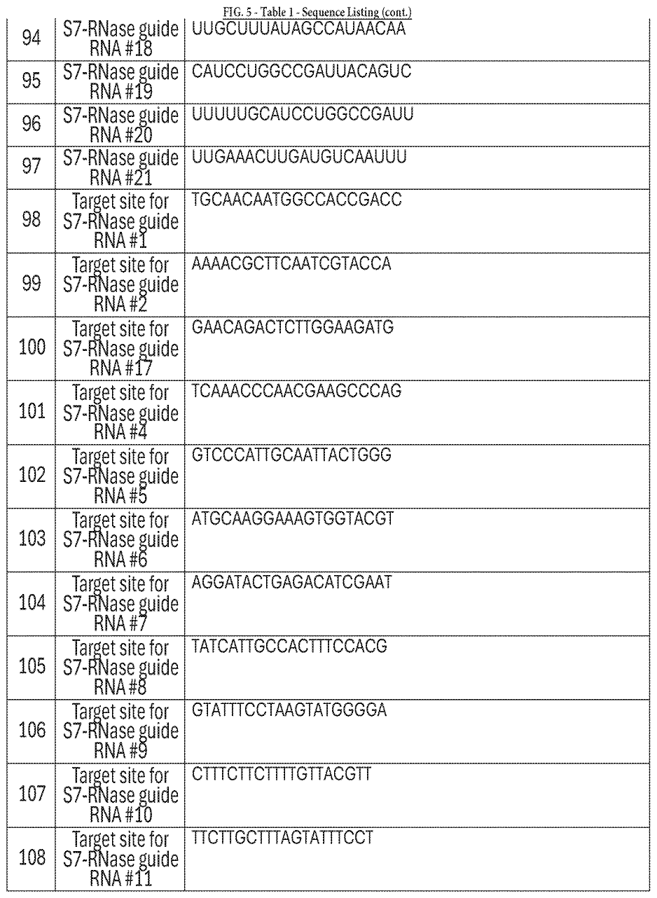

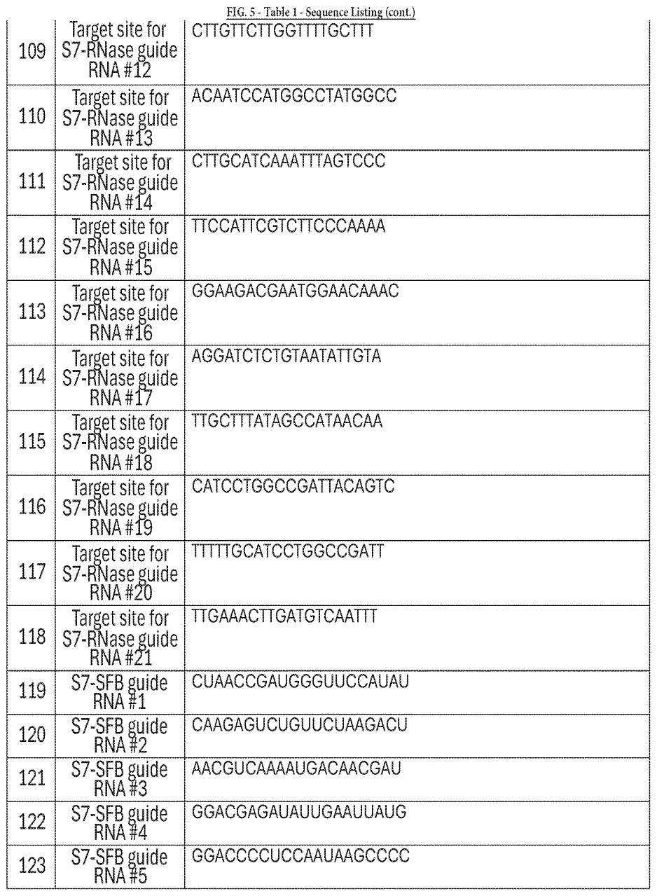

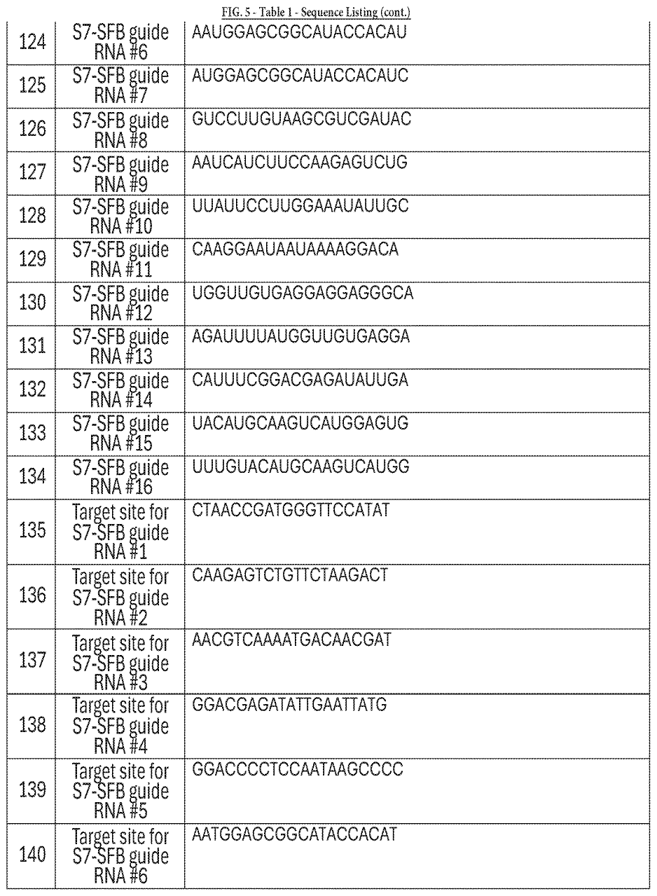

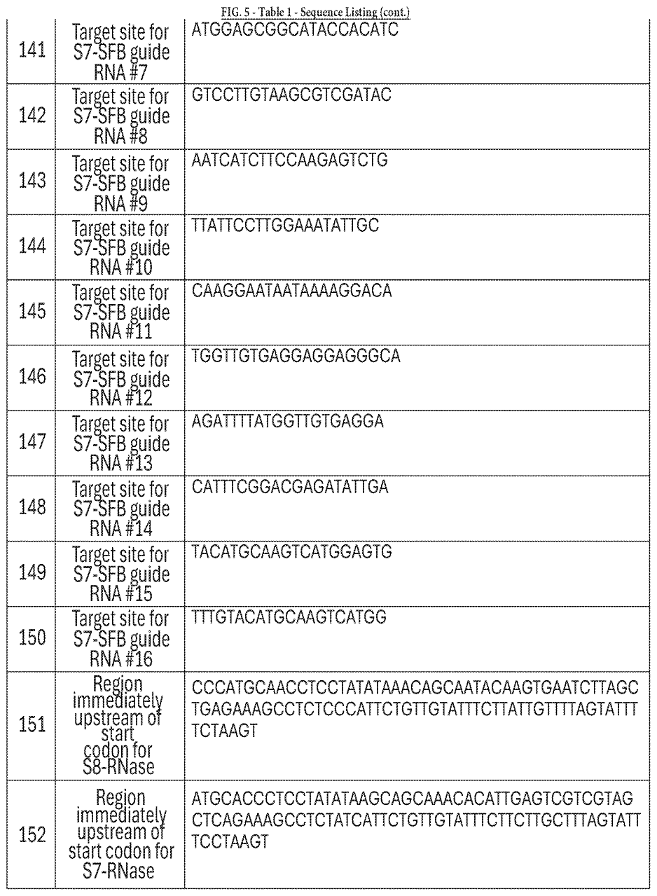

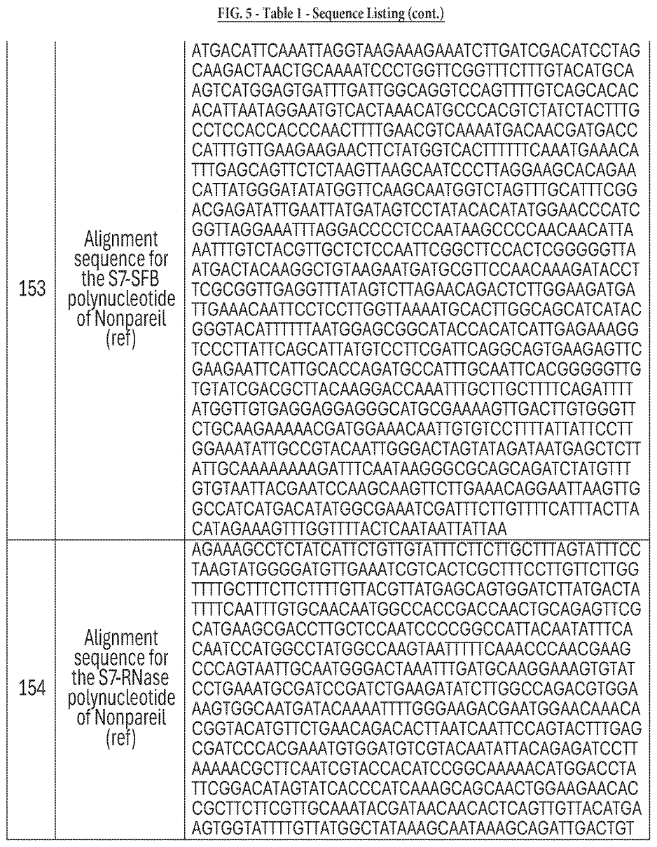

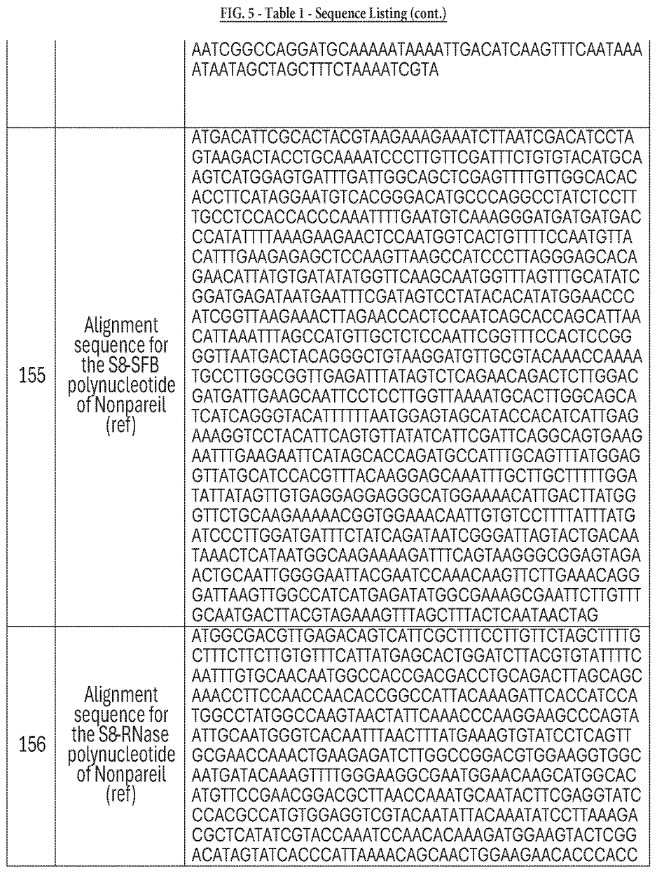

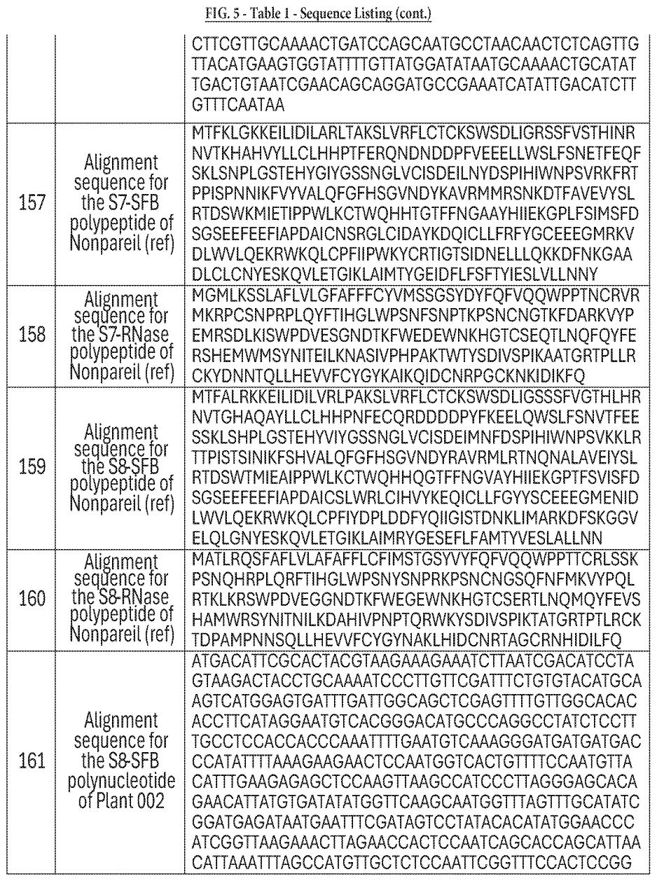

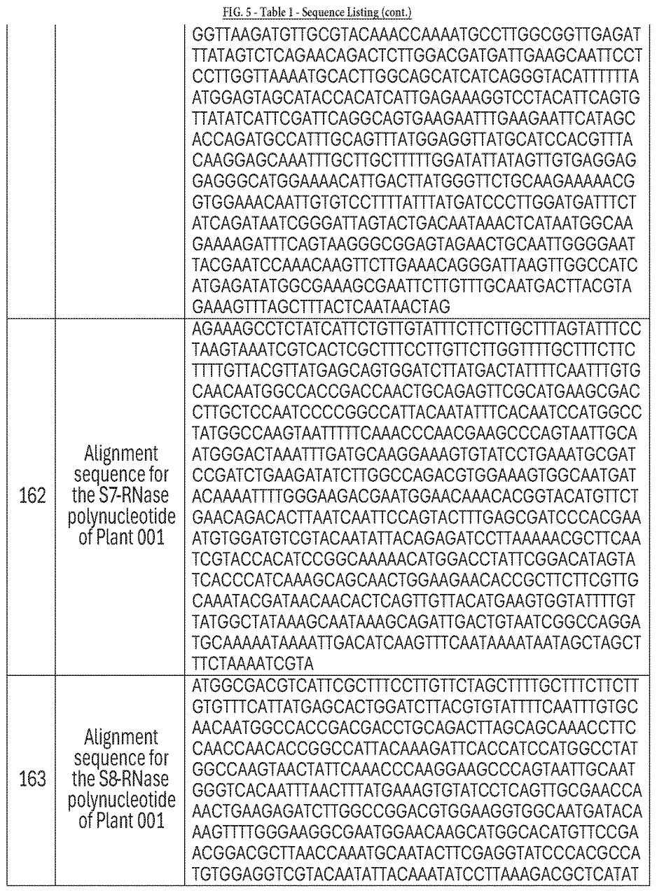

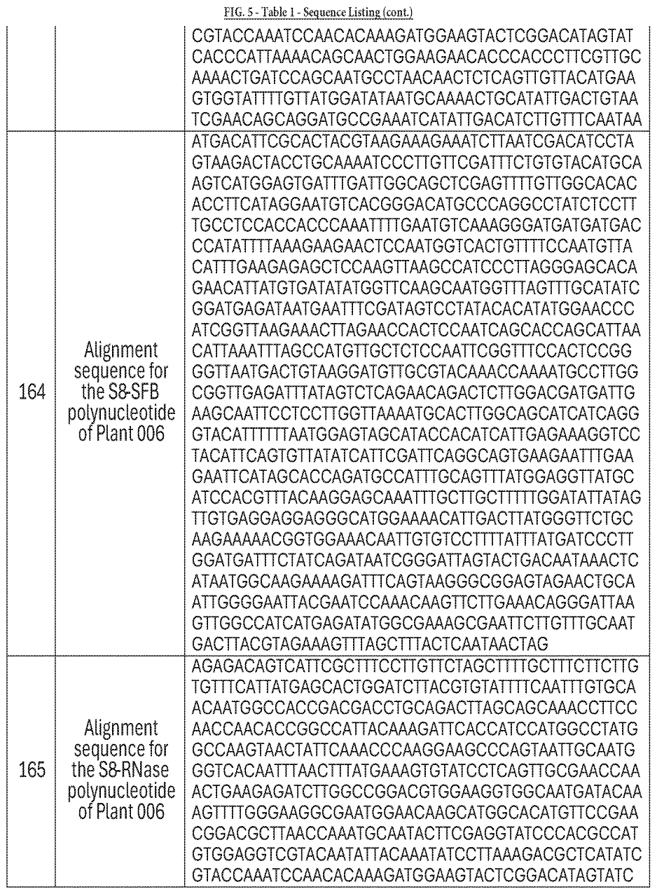

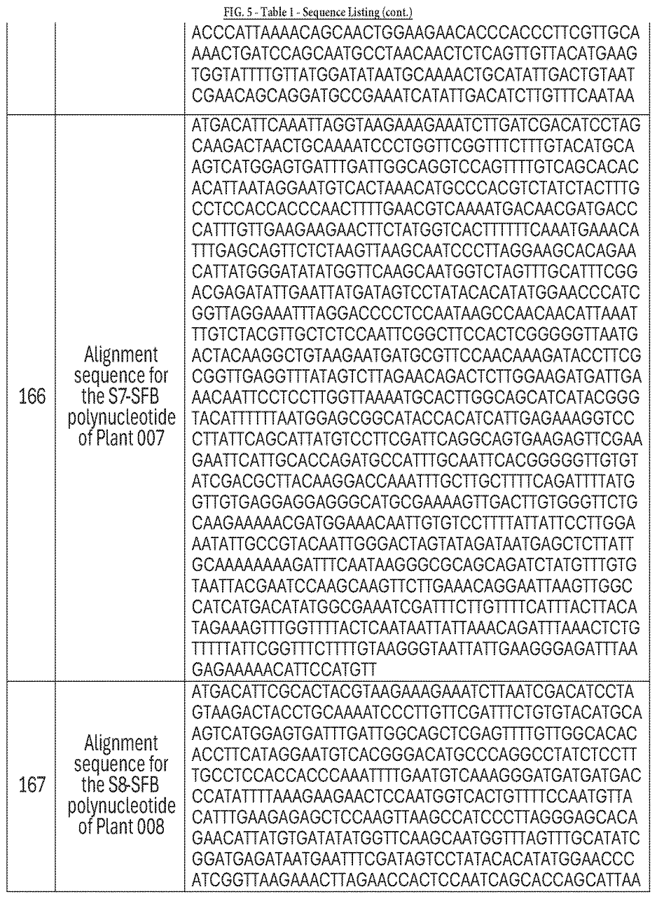

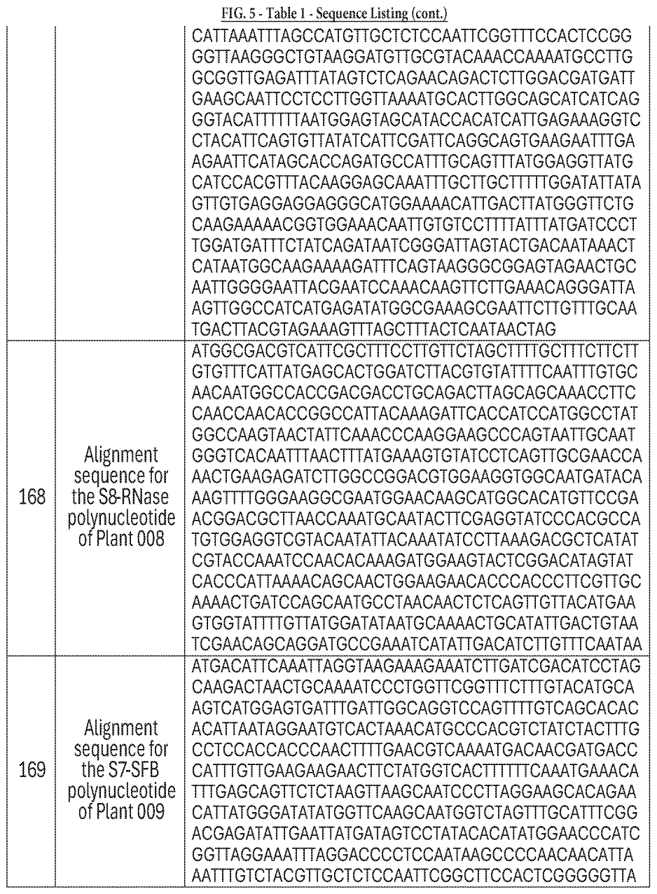

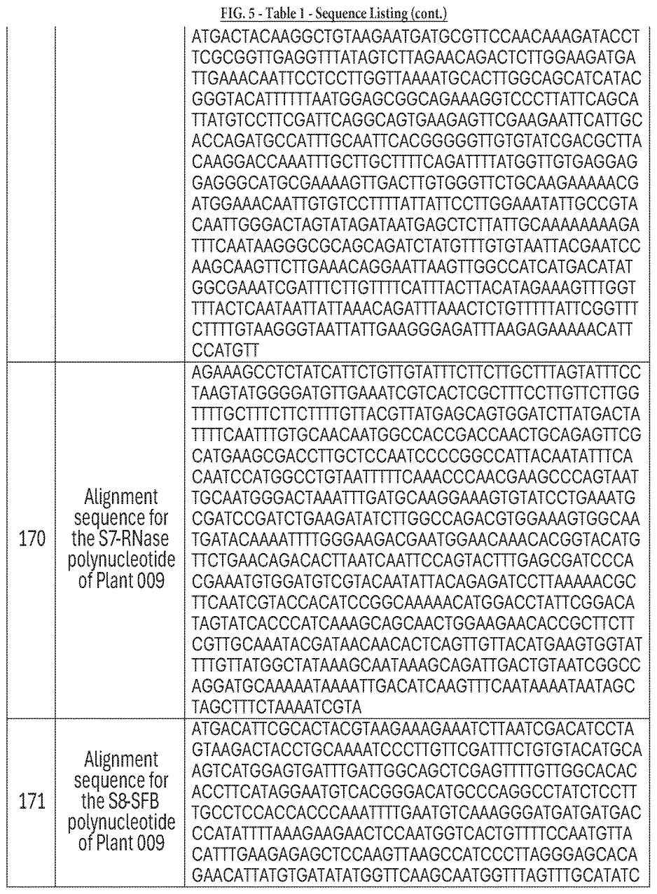

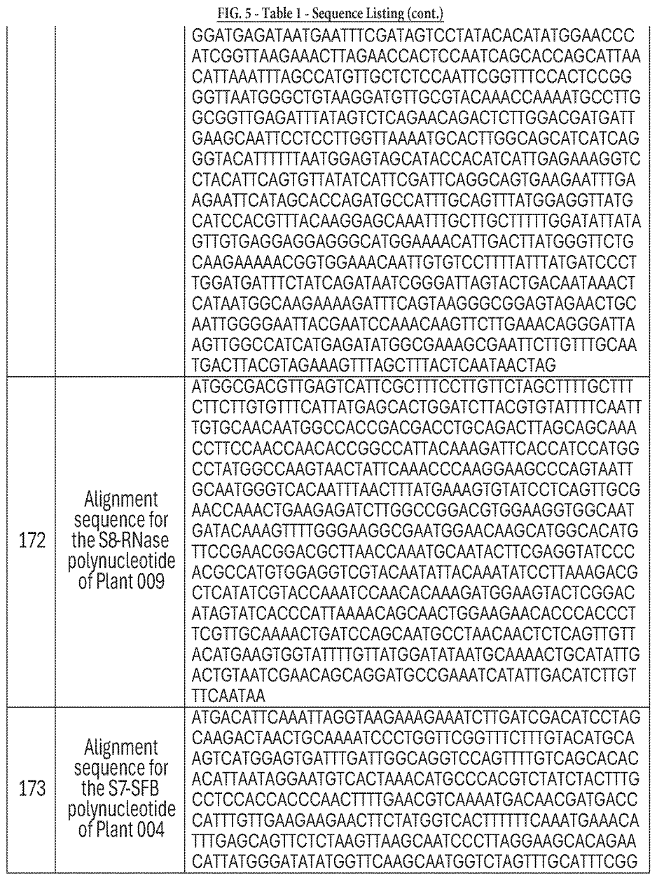

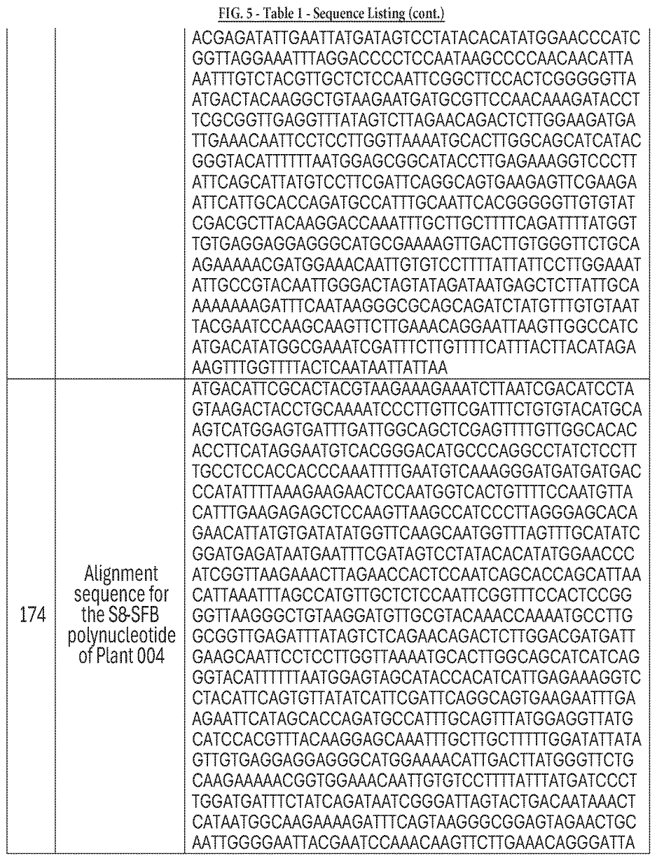

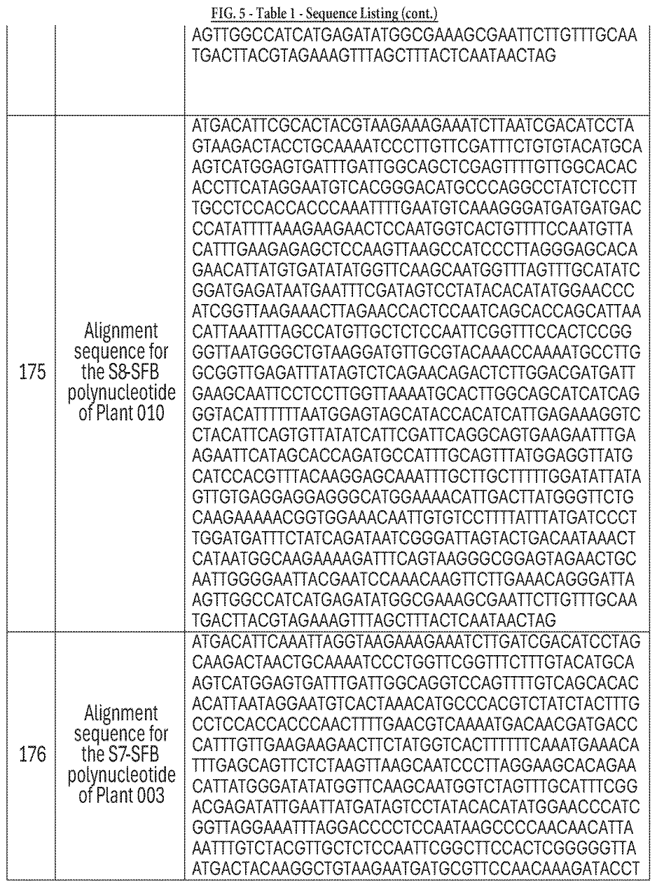

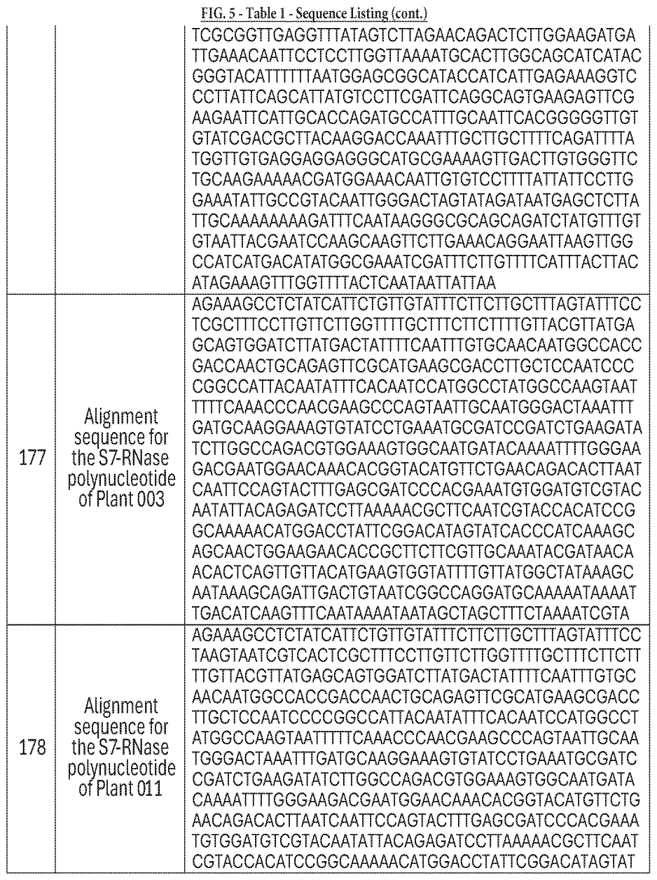

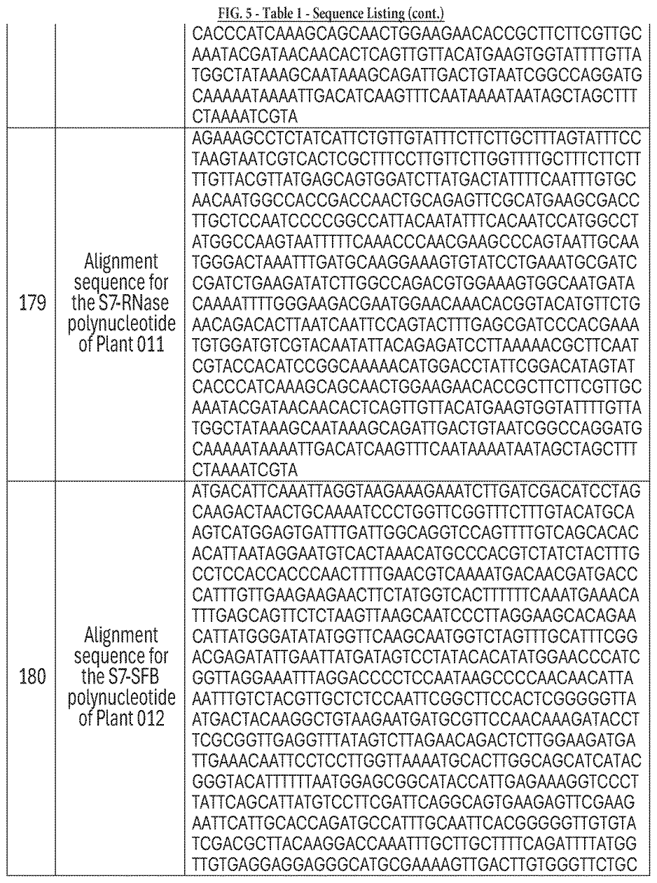

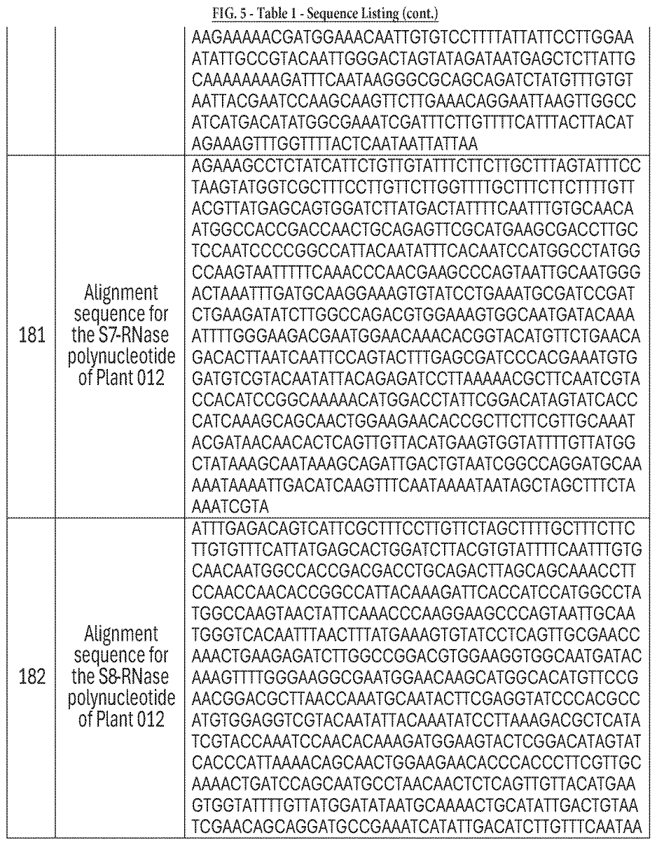

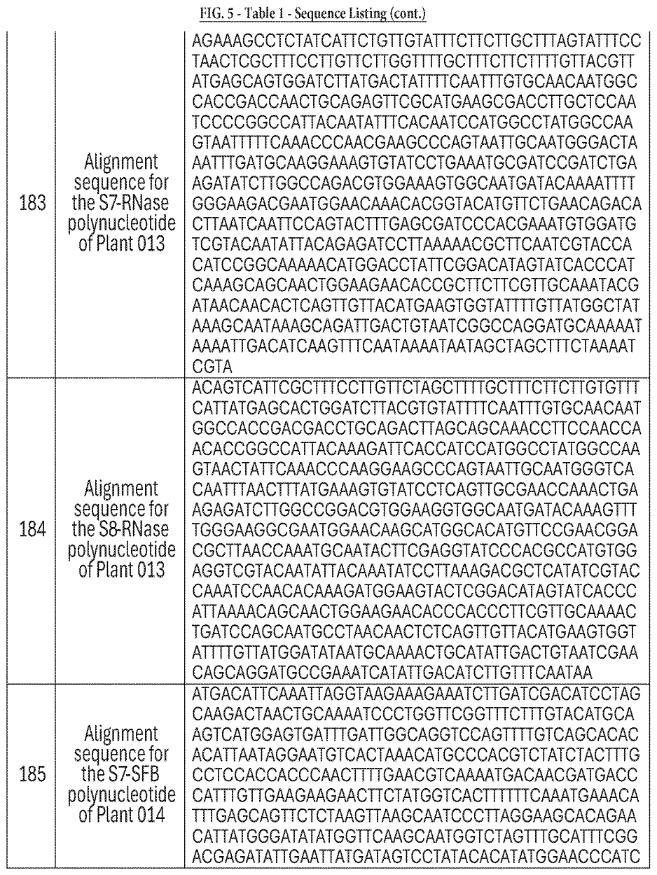

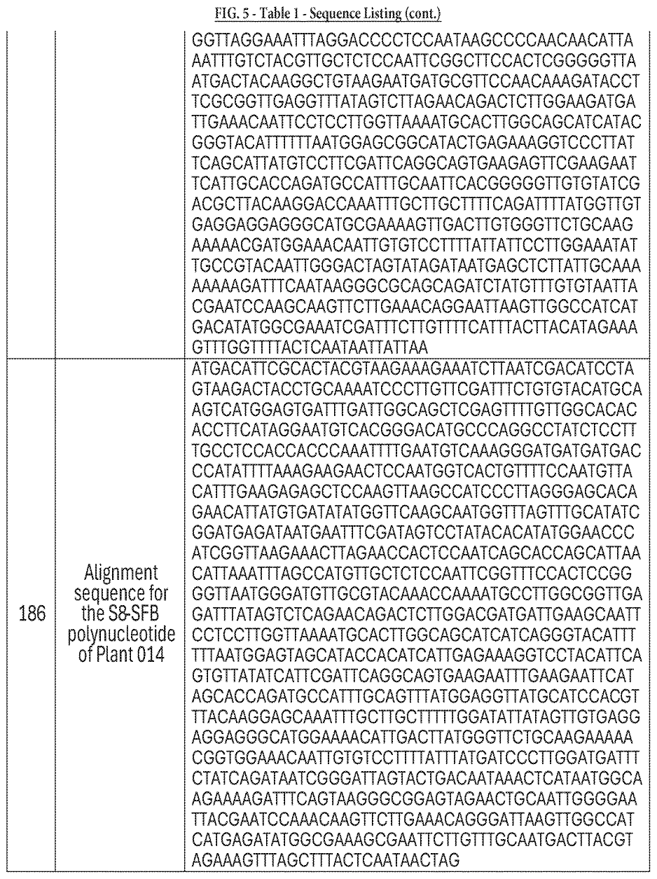

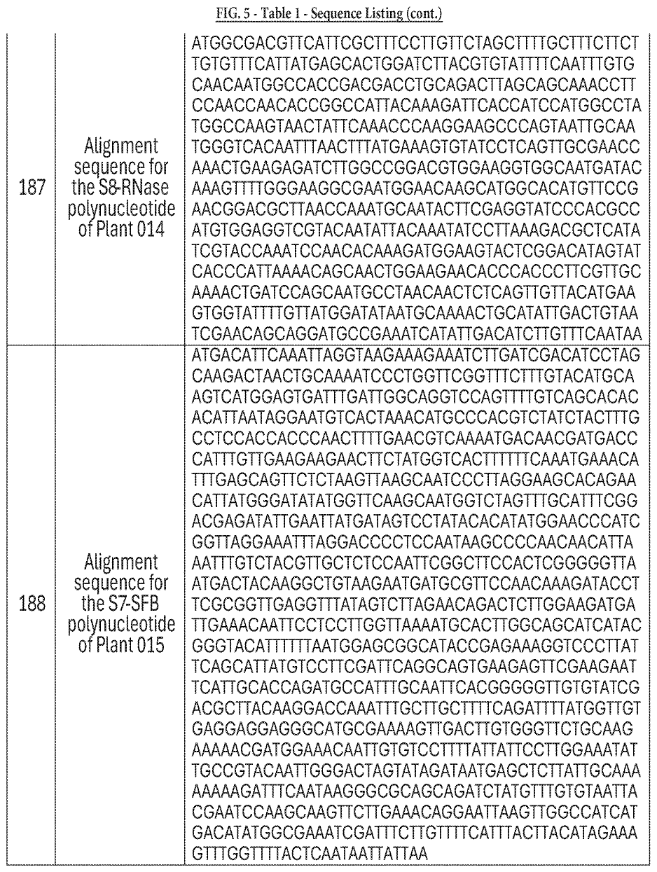

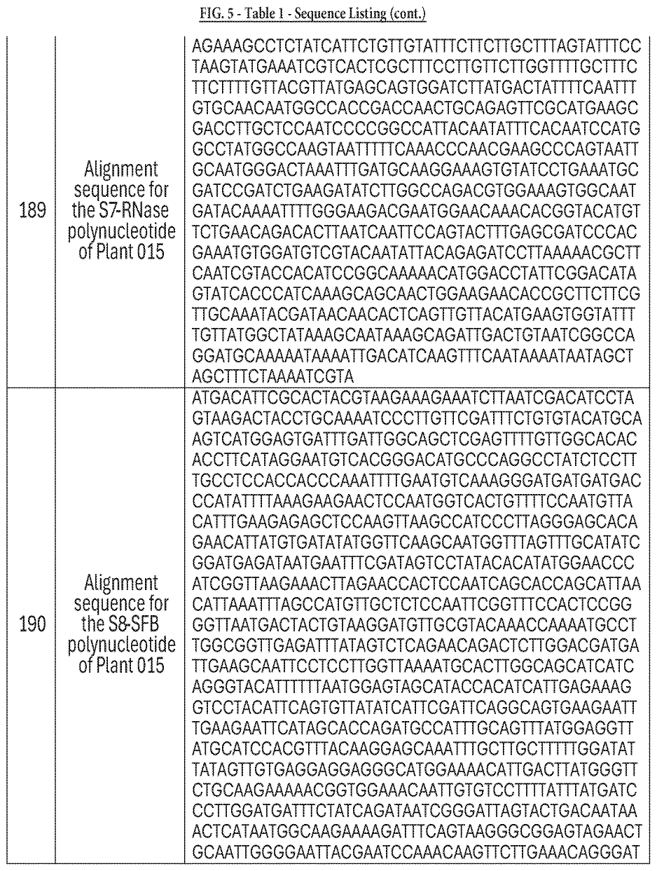

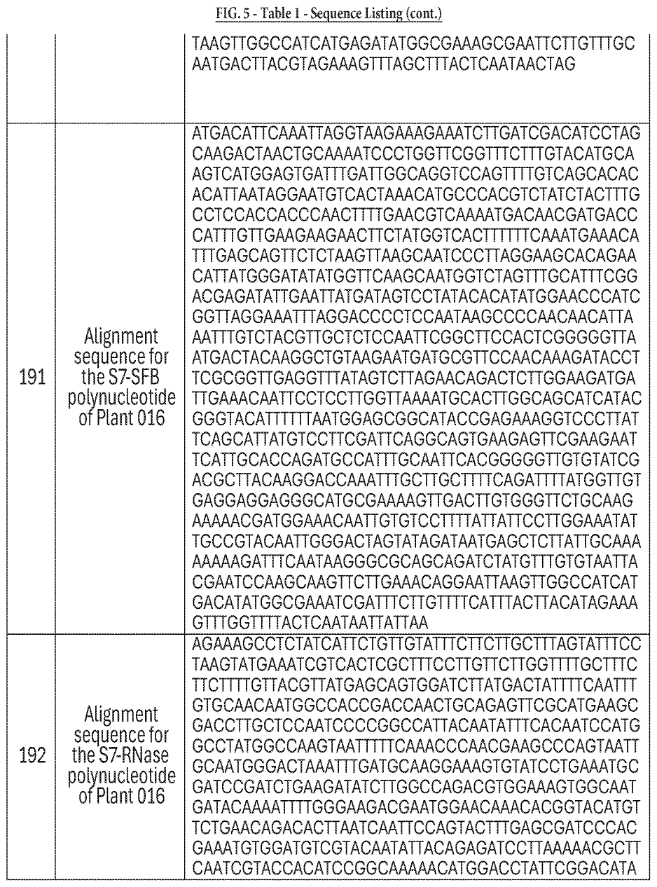

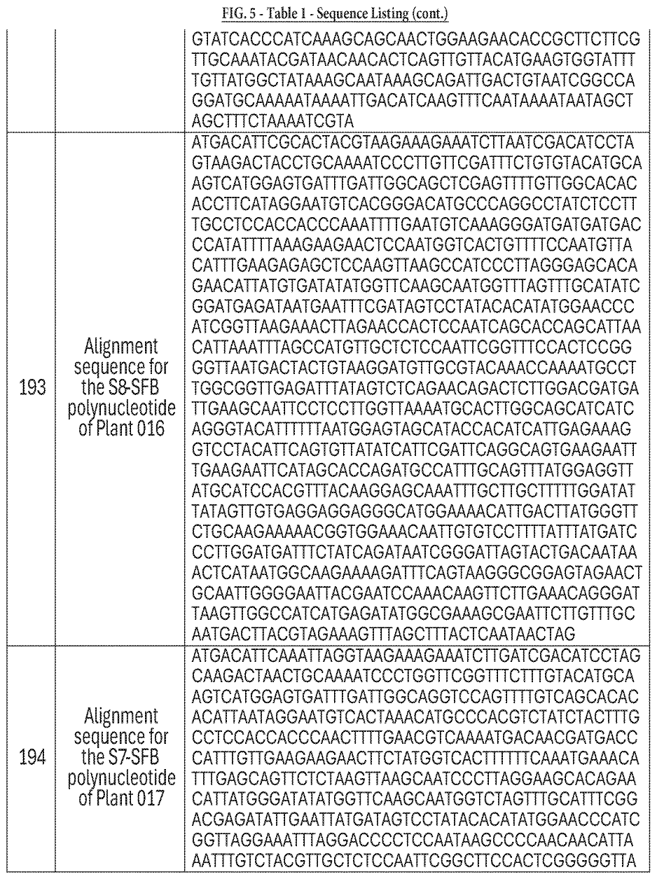

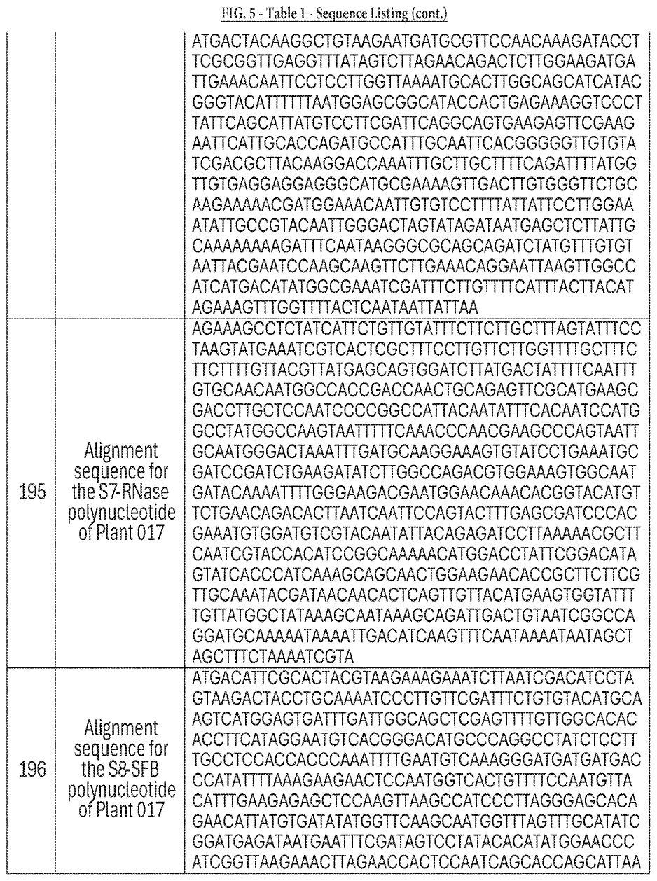

Sequences were then analyzed by aligning fastq files to reference sequences. Mutations adjacent to target PAM sites for each gene were tabulated relative to a control. Fastq data sets were quality filtered and aligned to the corresponding reference genomes using BWA mem, and variants were visualized and quantified using R scripts. Editing efficiency was calculated as the fraction of reads in an edited sample with on-target mutations adjacent to a PAM site relative to an unedited control. Editing efficiency was determined for each guide RNA. Editing efficiency for the crRNAs targeting S8-RNase are shown in FIG. 1. Editing efficiency for the crRNAs targeting S8-SFB are shown in FIG. 2. Editing efficiency for the crRNAs targeting S7-RNase are shown in FIG. 3. Editing efficiency for the crRNAs targeting S7-SFB are shown in FIG. 4. The error bars in FIGS. 1-4 represent the standard error of the mean, calculated by dividing the standard deviation (“SD”) by the square root of the sample size (“n”) (i.e., SD÷n²) and the dashed horizontal line represents a threshold of 20% editing efficiency.

The results depicted in FIGS. 1-4 reveal how difficult it is to find a guide RNA that results in a commercially-viable editing efficiency of at least approximately 20% in almond. At least 70 guides were tested in the course of developing the innovations disclosed herein and of those only 8 achieved or exceeded the 20% threshold, although one other (SEQ ID NO: 30) came very close to achieving the threshold. Furthermore, of the 8 that did meet or exceed the 20% threshold, three did so only barely (SEQ ID NOs: 92, 93, and 129). In a particularly competitive environment where a 25% threshold is needed to remain commercially viable, only three of the 70 guides would meet or exceed that threshold, translating to a success rate of less than 5%.

### Example 1E: Flow Cytometry

Flow cytometry was performed using the methods of Galbraith et al., 1983. SCIENCE 220, no. 4601:1049-1051. Briefly, intact nuclei were extracted into Galbraith buffer, filtered and stained with propidium iodide. DNA content of nuclei was determined by applying the samples to a BD Accuri C6 Flow Cytometer. Gating was performed and genomic DNA content was calculated by comparing the peak area for the sample to the known position of the internal control.

### Example 2: Edited ‘Nonpareil’ Almond Plants

### Example 2A: Plant Materials and Protoplast Isolation

Prunus dulcis var. ‘Nonpareil’ tissue culture shoots (herein referred to as “explants”) were provided by a local almond nursery. Upon receiving, explants were indexed and analyzed to confirm they were free of microorganisms. Tissue culture shoots can be maintained in vitro on suitable tissue culture media (for example, as described by McCown & Lloyd, 1981. HORTSCIENCE, 16, 453-453). For this experiment, tissue culture shoots were maintained in vitro on a media formulation essentially similar to Woody Plant Medium (WPM) (McCown & Lloyd, 1981) and supplemented with 20 g/L glucose, 0.7 mg/L 6-BAP, and 0.01 mg/L IBA and grown under 16 hour day with 90 μmol/m²s.

Side shoots, no more than 3 mm in height, were removed from two-week-old explants under aseptic conditions. Shoot tissue was sliced into thin sections 1 mm in width. Approximately 0.3 g of sliced shoot tissue was then placed in 10 ml of digest solution containing 20 g/L pectinase and 10 g/L cellulase RS (obtained from Yakult, Tokyo, 105-8664 Japan). The molarity of the digest solution was adjusted to 0.6 M using mannitol. Sliced tissue was then incubated in the digest solution for 14-16 hours at 20-24 °C.

The following day protoplasts were liberated from the sliced shoot tissue with 15 minutes of shaking at 30 rpm at 20 °C. Protoplasts were harvested through 40 micron sterile cell filters into sterile 50 mL conical tubes. Wash solution was added to a final volume of 30 ml. Protoplast solution was then centrifuged at 200×g for 5 minutes. Supernatant was removed and replaced with 2 mL of wash solution. Cells were gently resuspended by rocking and slowly layered onto a 21% sucrose solution. Tubes were centrifuged at 200×g for 10 minutes. A thick dark band of protoplasts appeared at the interface of the two solutions. This band was harvested in one continuous motion using a sterile serological pipette. Wash solution was added to a final volume of 30 ml. Protoplast solution was once again centrifuged at 200×g for 5 minutes. Supernatant was removed and replaced with 2 mL of wash solution. Cells were then quantified using a Burker hemocytometer and stored at 4 °C in the dark until transfection. A sample was reserved to test cell viability using FDA staining as described by Larkin, 1976, Planta 128, no. 3:213-216.

### Example 2B: Transfection

Transfection was carried out in a similar fashion to the methods described in Revilla et al., 1987, Plant Science, Volume 50, Issue 2, Pages 133-137. Protoplasts were centrifuged at 200×g for 10 minutes and resuspended in a 1× volume of MMG solution (mannitol/MgCl₂/MES, pH 6.0) to achieve a cell density of 1×10⁶ protoplasts/mL. Twenty microliters of freshly prepared RNP were added to the bottom of a 15 mL round bottom tube and 100 μL of protoplasts suspended in the transformation buffer, then mixed with the RNP solution. Next, 120 μL of PEG solution (as described in Revilla et al., 1987) was added and gently mixed by rotating the tubes. After a 15-minute incubation at room temperature, the cells were washed with PEG solution twice following centrifugation at 200×g for 10 minutes using a wash solution.

### Example 2C: Encapsulation and Regeneration of Plants

Encapsulation was achieved by combining an equal volume of transfected cells with a 3.2% sodium alginate solution. Between 100-150 μL of the resulting solution was pipetted using a wide bore 1000 μL pipette tip and dispensed on solid media containing 0.4 M sorbitol, 50 mM CaCl2, and 6 g of agar. The resulting solutions solidified into alginate lenses, encapsulating the transfected protoplasts. Alginate lenses were incubated for 20 minutes at room temperature before removing for regeneration.

Plant regeneration methods were based on the methods described in Nicolia et al., 2015, Targeted gene mutation in tetraploid potato through transient TALEN expression in protoplasts. J Biotechnol. 204:17-24. After encapsulation, alginate lenses were transferred to 12 well plate with 2.5 mL of Nicolia culture media E with 1 mg/L NAA and 0.4 mg/L BAP, and then placed at 24 °C in the dark. Alginate lenses remain in culture media for two weeks during which time cell division and signs of micro callus induction are observed through an inverted microscope.

After two weeks, half of the culture media is removed and replaced with a modified culture media without mannitol and 0.1 mg/L NAA and 0.4 mg/L BAP. After four weeks or when microcalli reach approximately two mm in diameter, alginate lenses were placed on shoot regeneration media. Calli were transferred every two to three weeks to fresh medium until shoots emerged after about 3 months.

When shoots reached a size of approximately 1 cm, they were excised from the callus and transferred to a media formulation similar to WPM and supplemented with 20 g/L glucose and 1 mg/L 6-BAP grown under 16 hour day with 90 μmol/m²s. Plants were multiplied with nodal segments and genotyped using the same sequencing methods described herein. Plants containing the desired genetic deletions were further multiplied. The five plants that survived these treatments were labeled 001-004 and 011, those that did not survive were labeled 006-010 and 012-017.

Genotyping results revealed that spontaneous tetraploidy had occurred in several of the plants/shoots derived from a single callus during the process and that two shoots from the same callus comprised different deletions in their S7-RNase, resulting in Plant 003 and Plant 011.

### Example 2E: Results

### Plant 001

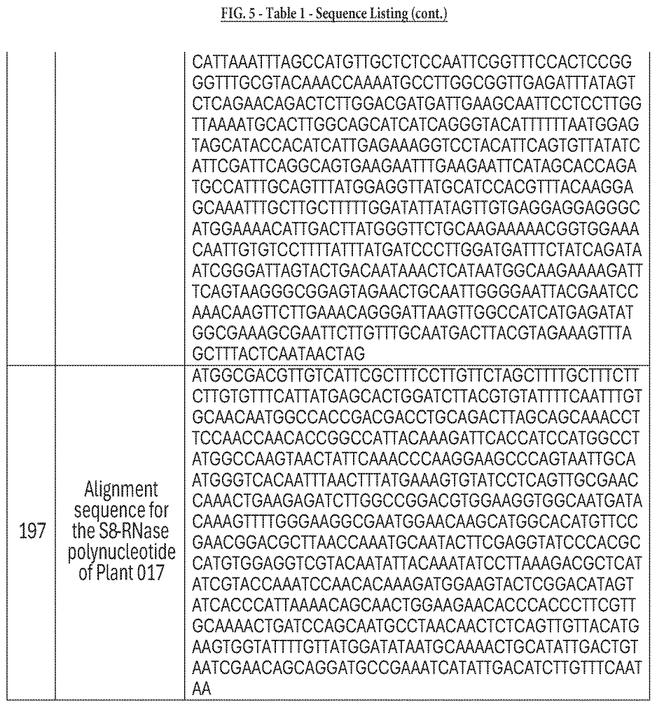

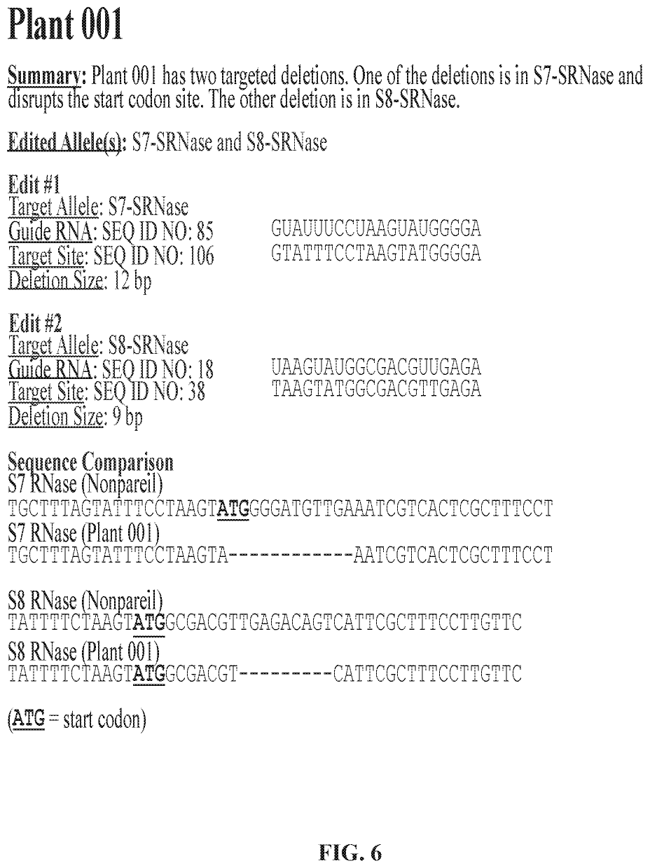

As shown in FIG. 6, Plant 001 has two targeted deletions. One of the deletions is in S7-RNase and disrupts the start codon site. The other deletion is in S8-RNase. Edited Allele(s): S7-RNase and S8-RNase

Edit #1
Guide RNA:
SEQ ID NO: 85
GUAUUUCCUAAGUAUGGGGA
Target Site:
SEQ ID NO: 106
GTATTTCCTAAGTATGGGGA
Deletion Size: 12 bp
Edit #2
Target Allele:
S8-RNase
Guide RNA:
SEQ ID NO: 18
UAAGUAUGGCGACGUUGAGA
Target Site:
SEQ ID NO: 38
TAAGTATGGCGACGTTGAGA
Deletion Size: 9 bp

### Plant 002

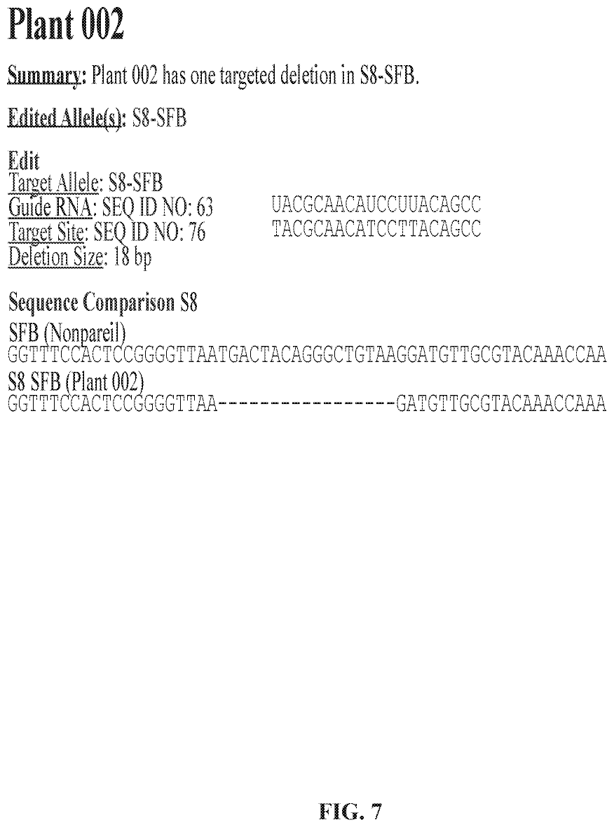

As shown in FIG. 7, Plant 002 has one targeted deletion in S8-SFB. Edited Allele(s): S8-SFB

Edit
Target Allele:
S8-SFB
Guide RNA:
SEQ ID NO: 63
UACGCAACAUCCUUACAGCC
Target Site:
SEQ ID NO: 76
TACGCAACATCCTTACAGCC
Deletion Size: 18 bp

### Plant 003

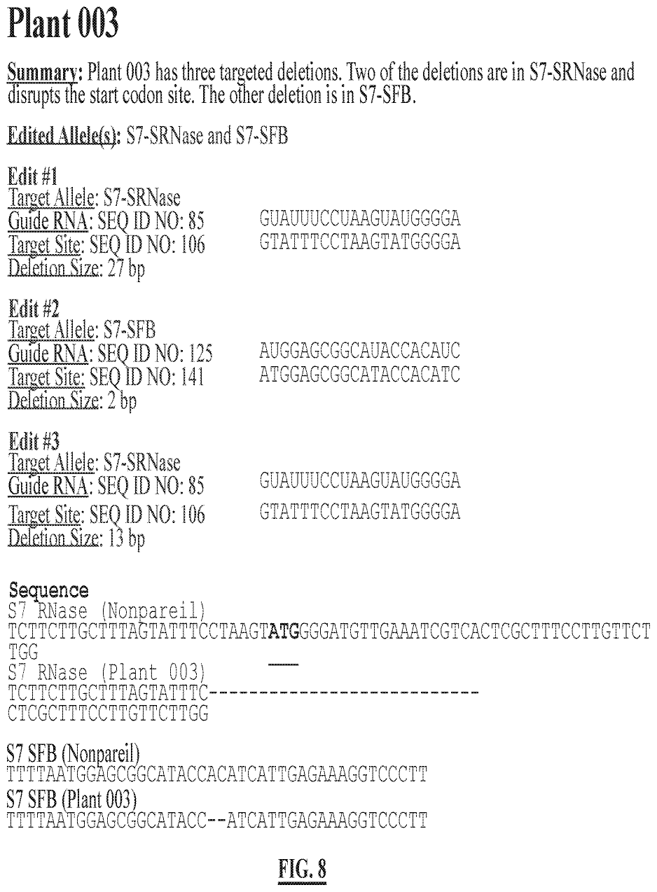

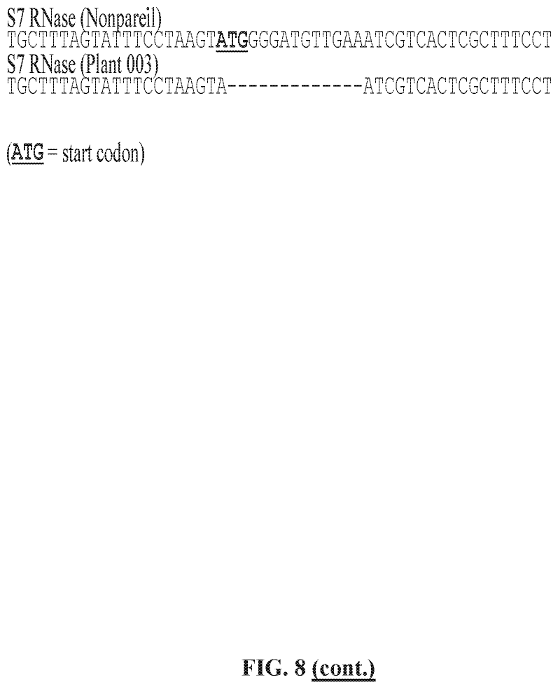

As shown in FIG. 8, Plant 003 has three targeted deletions. Two of the deletions is in S7-RNase and disrupts the start codon site. The other deletion is in S7-SFB. Edited Allele(s): S7-RNase and S7-SFB

Edit #1
Target Allele:
S7-RNase
Guide RNA:
SEQ ID NO: 85
GUAUUUCCUAAGUAUGGGGA
Target Site:
SEQ ID NO: 106
GTATTTCCTAAGTATGGGGA
Deletion Size: 27 bp
Edit #2
Target Allele:
S7-SFB
Guide RNA:
SEQ ID NO: 125
AUGGAGCGGCAUACCACAUC
Target Site:
SEQ ID NO: 141
ATGGAGCGGCATACCACATC
Deletion Size: 2 bp
Edit #3
Target Allele:
S7-SRNase
ATGGAGCGGCATACCACATC
Guide RNA:
SEQ ID NO: 85
ATGGAGCGGCATACCACATC
Target Site:
SEQ ID NO: 106
UAUUUCCUAAGUAUGGGGA
Deletion Size: 13 bp

### Plant 004

As shown in FIG. 9, Plant 004 has two targeted deletions. One of the deletions is in S7-SFB. The other deletion is in S8-SFB. Edited Alleles: S7-SFB and S8-SFB

Edit #1
Target Allele:
S7-SFB
Guide RNA:
SEQ ID NO: 125
AUGGAGCGGCAUACCACAUC
Target Site:
SEQ ID NO: 141
ATGGAGCGGCATACCACATC
Deletion Size: 6 bp
Edit #2
Target Allele:
S8-SFB
Guide RNA:
SEQ ID NO: 57
CACUCCGGGGUUAAUGACUA
Target Site:
SEQ ID NO: 70
CACTCCGGGGTTAATGACTA
Deletion Size: 8 bp

### Plant 011

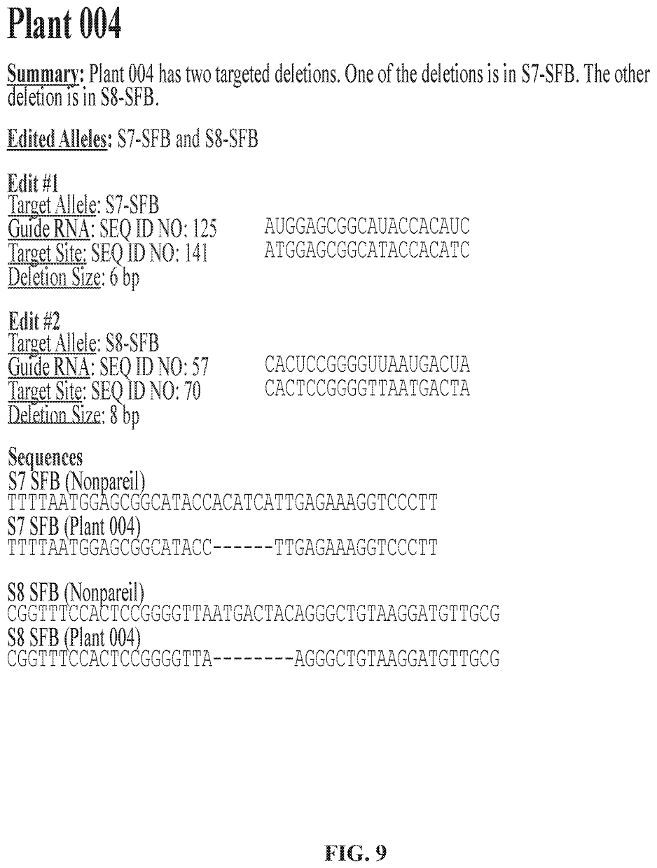

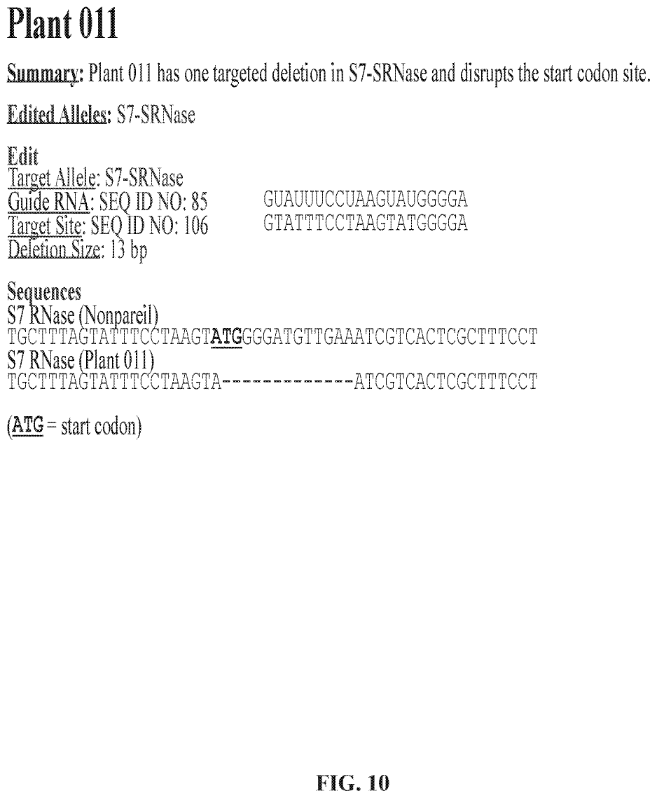

As shown in FIG. 10, Plant 011 has one targeted deletion in S7-RNase and disrupts the start codon site. Edited Alleles: S7-RNase

Edit
Target Allele:
S7-RNase
Guide RNA:
SEQ ID NO: 85
GUAUUUCCUAAGUAUGGGGA
Target Site:
SEQ ID NO: 106
GTATTTCCTAAGTATGGGGA
Deletion Size: 13 bp

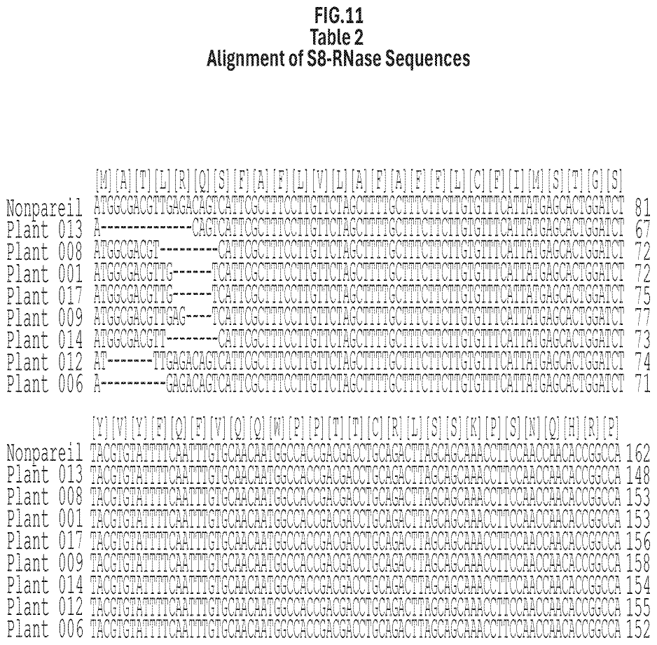

FIG. 11 provides a visualization of the alignment of S8-RNase sequences for Plant 013 (SEQ ID NO: 184), Plant 008 (SEQ ID NO: 168), Plant 001 (SEQ ID NO: 163), Plant 017 (SEQ ID NO: 197), Plant 009 (SEQ ID NO: 172), Plant 014 (SEQ ID NO: 187), Plant 012 (SEQ ID NO: 182), and Plant 006 (SEQ ID NO: 165).

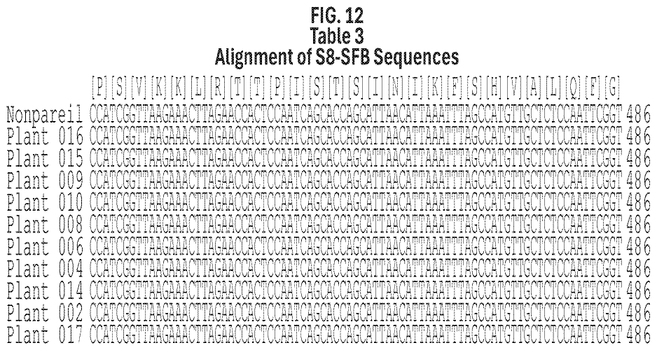

FIG. 12 provides a visualization of the alignment of Alignment of S8-SFB Sequences for Plant 016 (SEQ ID NO: 193), Plant 015 (SEQ ID NO: 190), Plant 009 (SEQ ID NO: 171), Plant 010 (SEQ ID NO: 175), Plant 008 (SEQ ID NO: 167), Plant 006 (SEQ ID NO: 164), Plant 004 (SEQ ID NO: 174), Plant 014 (SEQ ID NO: 186), Plant 002 (SEQ ID NO: 161), and Plant 017 (SEQ ID NO: 196)

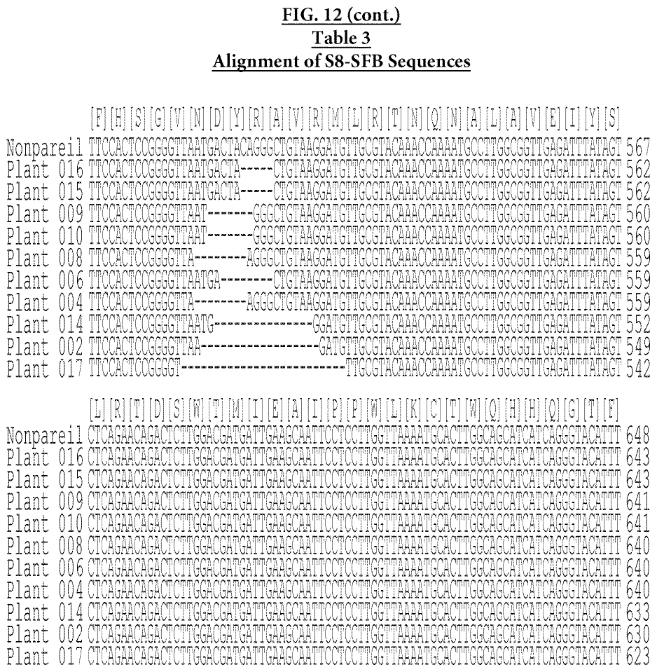

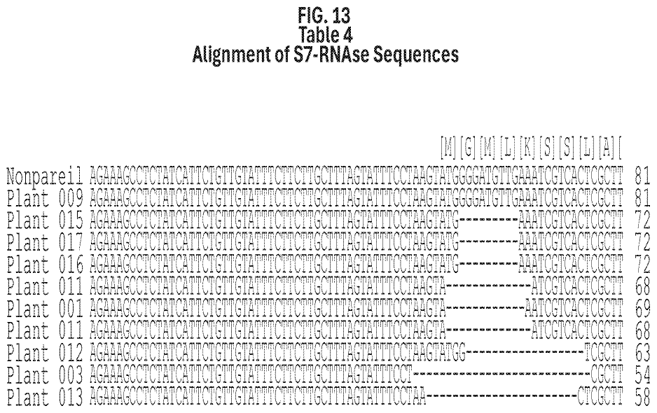

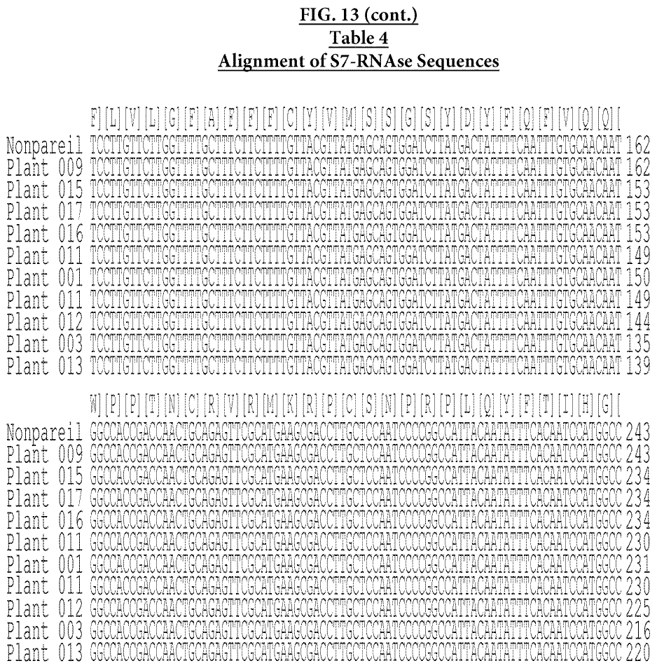

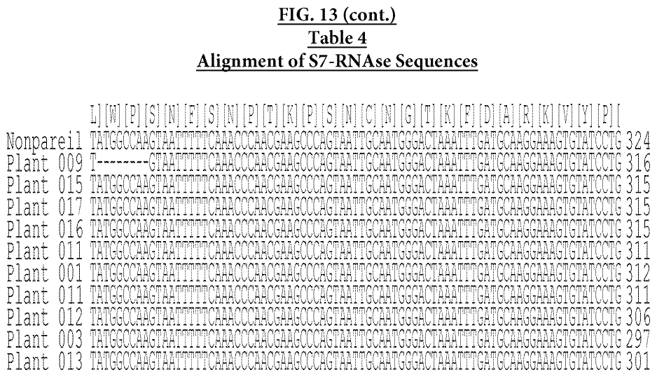

FIG. 13 provides Table 4, which provides an alignment of S7-RNAse sequences for Plant 009 (SEQ ID NO: 170), Plant 015 (SEQ ID NO: 189), Plant 017 (SEQ ID NO: 195), Plant 016 (SEQ ID NO: 192), Plant 011 (SEQ ID NO: 179), Plant 001 (SEQ ID NO: 162), Plant 011 (SEQ ID NO: 178), Plant 012 (SEQ ID NO: 181), Plant 003 (SEQ ID NO: 177), and Plant 013 (SEQ ID NO: 183).

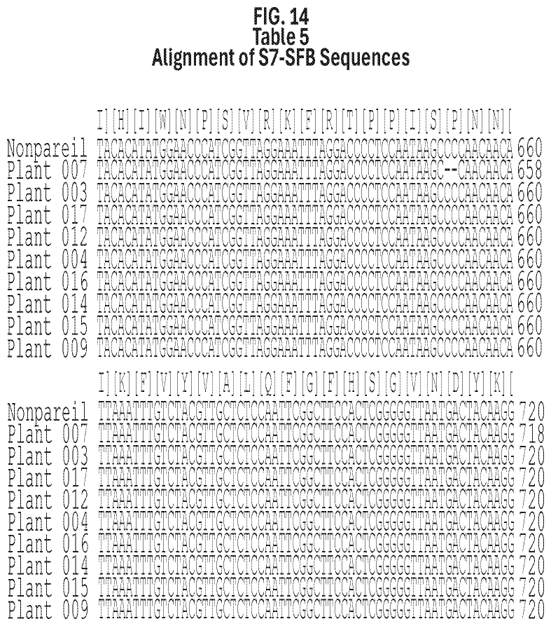

FIG. 14 provides Table 5, which provides an alignment of S7-SFB sequences for Plant 007 (SEQ ID NO: 166), Plant 003 (SEQ ID NO: 176), Plant 017 (SEQ ID NO: 194), Plant 012 (SEQ ID NO: 180), Plant 004 (SEQ ID NO: 173), Plant 016 (SEQ ID NO: 191), Plant 014 (SEQ ID NO: 185), Plant 015 (SEQ ID NO: 188), and Plant 009 (SEQ ID NO: 169).

### Example 3: Phenotypic Evidence of Self-Incompatibility Based on Bimolecular Fluorescence Complementation (BiFC) Tests Using Yellow Fluorescent Protein (Split YFP)

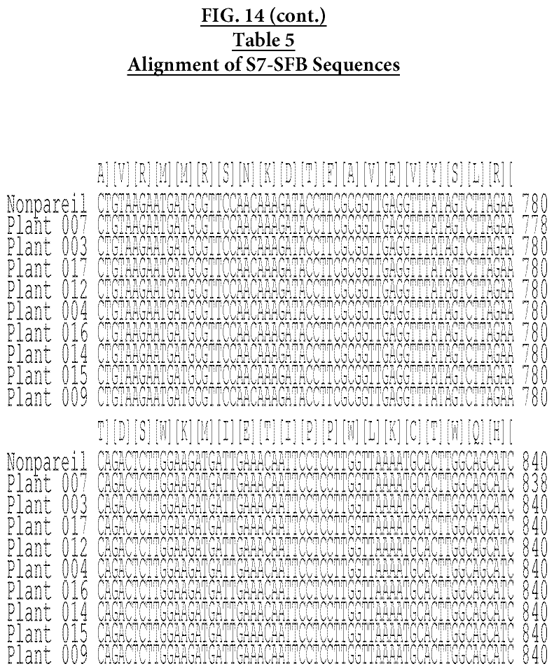

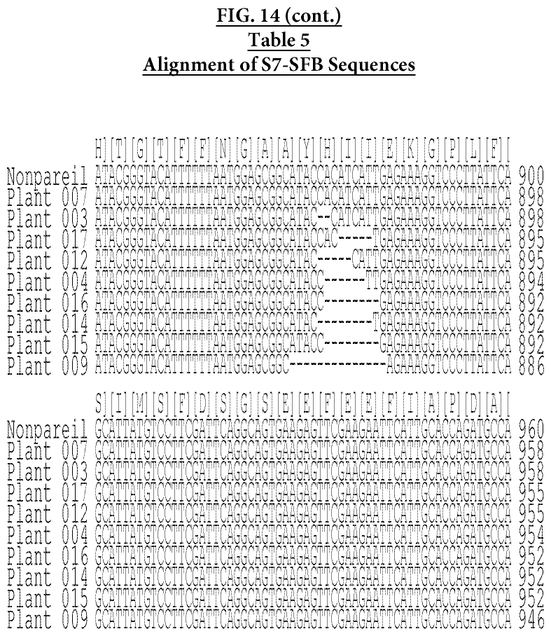

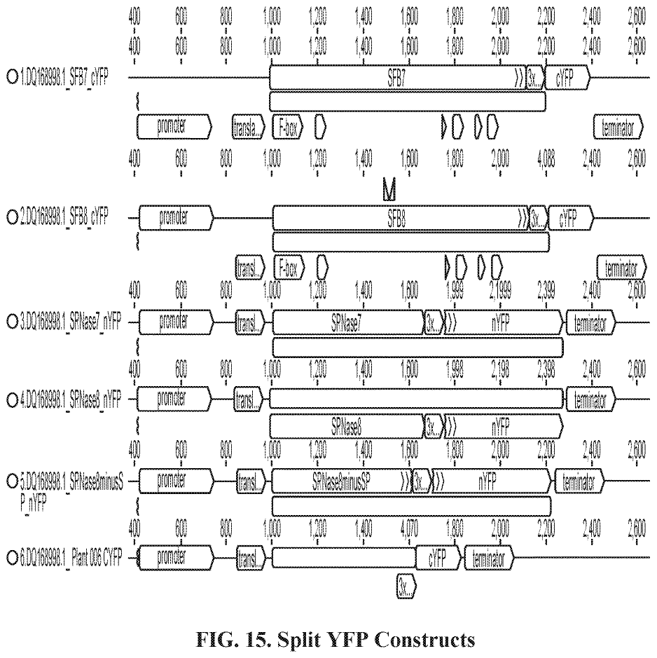

FIG. 15 depicts constructs used in this experiment. 3 uL of DNA at 1 ug/uL of each construct was combined to create the following construct combinations:
1. S7-SFB_cYFP and S7-RNase_nYFP
2. S8-SFB_cYFP and S8-RNase_nYFP
3. S8-SFB_cYFP and S8-RNaseMinusSP_nYFP
4. S8-SFB_Plant 006_cYFP and S8-RNase_nYFP
5. S8-SFB_Plant 006_cYFP and S8-RNase8MinusSP_nYFP
6. S7-SFB_cYFP and S8-RNase_nYFP (slightly less than 3 μL of the S8-RNase_nYFP was added to this combination)

6 μL of total plasmid DNA of each of the construct combinations were placed in separate 6-well plates. A 6-well control plate was also generated wherein the wells contained 6 μL of ddH20 instead of the plasmid DNA.

Next, Nonpareil almond cells were isolated and resuspended to 1,000,000 cells/mL in TWS prior to transfection. 100 μL of resuspended cells were added to the wells containing the plasmid DNA in a well plate and 110 μL of PEG was then added to each well. Washes were then performed with At-W5 and post transfection fluorescence signals were quantified after 24 hours and then again after 48 hours using a plate reader and SoftMax software 7.0 Protocol Manager Basic Endpoint Settings on new protocol Read modes. FL Excitation emission was 485, 535 and the plate type was a Corning 96-well Half-Area Microplate (landscape). PMT and Optics Settings Integration Time was set at 2000 ms.

### Results

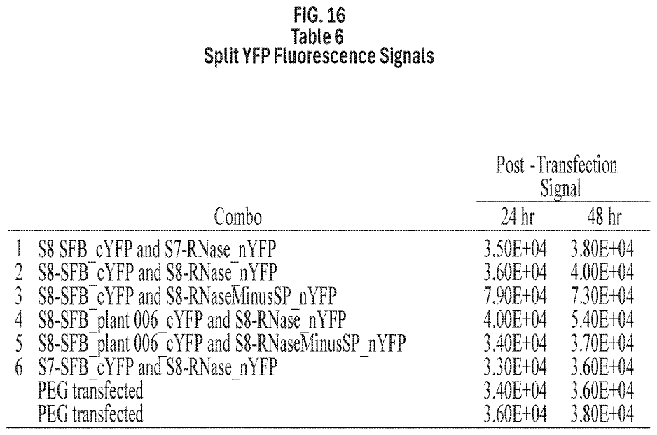

The fluorescence signals measured from each transfection are shown below in Table 6 and in FIG. 16.

TABLE 6

| | | Post-Transfection Signal | |
|---|---|---|---|
| | Combo | 24 hr | 48 hr |
| 1 | S8 SFB_cYFP and S7-RNase_nYFP | 3.50E+04 | 3.80E+04 |
| 2 | S8-SFB_cYFP and S8-RNase_nYFP | 3.60E+04 | 4.00E+04 |
| 3 | S8-SFB_cYFP and | 7.90E+04 | 7.30E+04 |
| | S8-RNasemMnusSP_nYFP | | |
| 4 | S8-SFB_Plant 006_cYFP | 4.00E+04 | 5.40E+04 |
| | and S8-RNase_nYFP | | |
| | S8-SFB_Plant 006_cYFP and S8- | | |
| 5 | RNaseMinusSP_nYFP | 3.40E+04 | 3.70E+04 |
| 6 | S7-SFB_cYFP and S8-RNase_nYFP | 3.30E+04 | 3.60E+04 |
| | PEG transfected | 3.40E+04 | 3.60E+04 |
| | PEG transfected | 3.60E+04 | 3.80E+04 |

The well containing S8-SFB_cYFP and S8-RNaseMinusSP_nYFP produced a signal that was obviously greater than the background. The well containing S8-SFB_Plant 006 cYFP and S8-RNase_nYFP produced a slight increase in signal relative to background and in combo 4 (S8-SFB_Plant 006_cYFP and S8-RNase_nYFP). Taken together, it appears that it is necessary to remove SP from S-RNase to get intracellular interaction with SFB in planta. It also appears that S8-SFB_Plant 006 is not binding to S8-RNase as strongly as S8-SFB(WT), considering the lower signal observed in combination 5 compared to combination 3.

### Example 4: Editing SFB and SRNase Alleles

Several almond plants with edited profiles in SFB and S-RNase alleles were generated. Plant 002, Plant 001, and Plant 006 are 2n shoots with edits. Plant 003 and Plant 013 are 4n edited shoots with edits. Edits made to the predicted S7-RNase start codon appear to cause substantial changes in S-RNase in vivo localization, possibly even eliminating synthesis entirely.

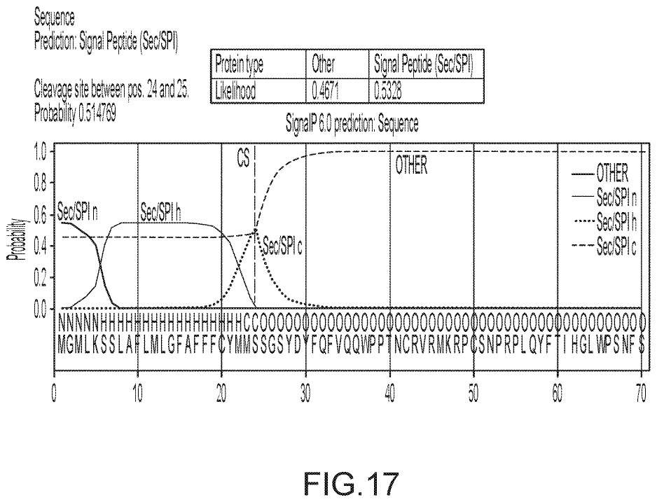

SignalIP 6.0 (Teufel F, Almagro Armenteros J J, Johansen A R, Gislason M H, Pihl S I, Tsirigos K D, Winther O, Brunak S, von Heijne G, Nielsen H. SignalP 6.0 predicts all five types of signal peptides using protein language models. Nat Biotechnol. 2022 July; 40(7):1023-1025. doi: 10.1038/s41587-021-01156-3. Epub 2022 Jan. 3. PMID: 34980915; PMCID: PMC9287161) predicted that S7-RNase has a signal peptide with a cleavage site between amino acids 24 and 25 (FIG. 17).

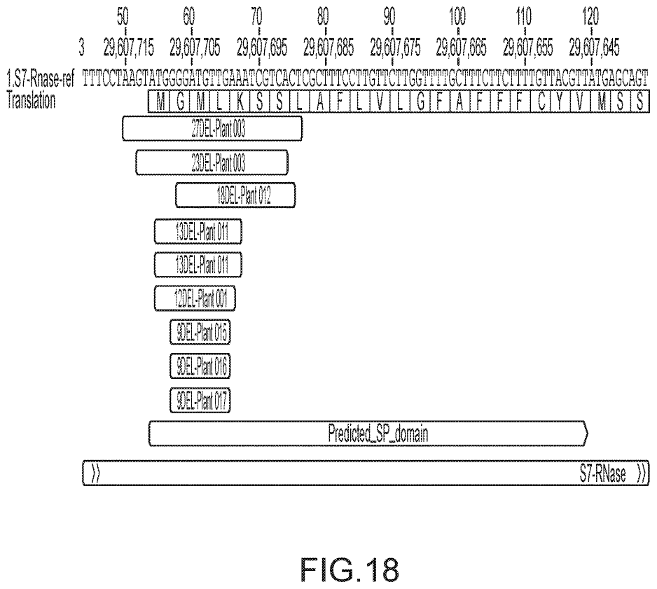

Edited alleles in regenerated shoots (Plant 003, Plant 013, Plant 001, and Plant 011) eliminated the first and second predicted methionines and potential start codons for S7-RNase. However, the third potential start codon in the S7-RNase sequence remains, and this third methionine appears right before the predicted cleavage site for the signal peptide sequence of SRNase7 (FIG. 18).

In the case that this third start codon was functional to initiate protein synthesis in the absence of the first two, we tested if there would be a change in the resulting protein's subcellular localization by designing constructs to test S7-RNase localization with and without the predicted signal peptide (SP) sequence (first 22 amino acids). Constructs were synthesized by Twist Bioscience and cloned into their pTwist Amp High Copy vector. We used single 35S promoter and 35S terminator sequences to drive transcription of S7-RNase (WT)+3×HA+fullYFP and S7-RNase-SP (minus signal peptide)+3×HA+fullYFP sequences. Escherichia coli were transformed with plasmid DNA encoding the constructs by Twist Bioscience. Plasmid DNA was isolated from E. coli following Qiagen's Plasmid Midi Kit and protocol (Ref. 12143).

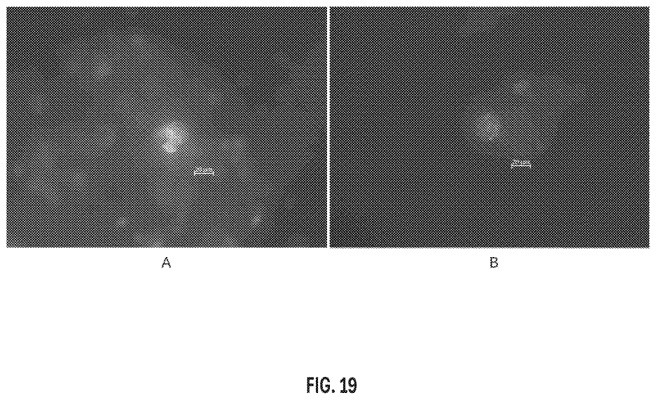

Over two experiments, protoplasts from almond plants following established protocols were isolated and transfected with the plasmid DNA described herein following the conditions outlined in a published protocol (Clasen et al. (2016). Improving cold storage and processing traits in potato through targeted gene knockout. Plant biotechnology journal, 14 (1), 169-176; Fossi et al. (2019). Regeneration of Solanum tuberosum plants from protoplasts induces widespread genome instability. Plant physiology, 180 (1), 78-86; Masson et al. (1987). Plant regeneration from protoplasts of diploid potato derived from crosses of Solanum tuberosum with wild Solanum species. Plant Science, 53(2), 167-176; Nicolia et al. (Nicolia et al. (2015) Targeted gene mutation in tetraploid potato through transient TALEN expression in protoplasts. Journal of biotechnology. 204:17-24; Veillet et al. (2019); Yao, L., Liao, X., Gan, Z., Peng, X., Wang, P., Li, S., & Li, T. (2016). Protoplast isolation and development of a transient expression system for sweet cherry (Prunus avium L.). Scientia Horticulturae, 209, 14-21). The Solanum tuberosum GBSSI gene: a target for assessing gene and base editing in tetraploid potato. Plant cell reports, 38(9), 1065-1080). After approximately 24 hours post transfection, cells were visualized using an Axiovert 200 M microscope (Zeiss) with filter set 46 HE. Distinct localization patterns between the two samples were observed in both experiments. S7-RNase_fullYFP samples showed diffuse YFP signal, whereas S7-RNase-SP_fullYFP samples showed distinct puncta (FIG. 19). We hypothesize that these dramatic differences in subcellular localization between the two proteins in vivo support the assertion that the edited almond shoots with the first two potential start codons of S7-RNase removed (Plant 003, Plant 013, Plant 001, and Plant 011) will have clear biological changes relative to those with unedited S7-RNase.

The embodiments further relate to fresh food, food products and processed food produced from almonds from a self-fertile almond plant or almond genome comprising a deletion of one or more genes within the one or more S-locus haplotypes as described herein, including seed, nuts, butters, oils, milk, cosmetics, extract, isolate, food, livestock feed, and a raw material for industry, etc.

What is claimed is:

1-86. (canceled)

87. A self-fertile almond plant comprising one or more S-locus haplotypes,
wherein the one or more S-locus haplotypes comprise a deletion that results in a frameshift mutation or an in-frame mutation in one or more S-locus genes.

88. The self-fertile almond plant of claim 87, wherein said deletion comprises:
a deletion of an S8-RNase gene with a transcribed portion, wherein the transcribed portion comprises a DNA sequence with at least 90%, at least 95%, at least 99% or 100% sequence identity to SEQ ID NO: 1;
a deletion of an S8-RNase gene comprising a DNA protein coding sequence with at least 90%, at least 95%, at least 99% or 100% sequence identity to SEQ ID NO: 2;
a deletion of a gene encoding an S8-RNase amino acid sequence with at least 90%, at least 95%, at least 99% or 100% sequence identity to SEQ ID NO: 3;
a deletion of an S8-SFB gene with a transcribed portion, wherein the transcribed portion comprises a DNA sequence with at least 90%, at least 95%, at least 99% or 100% sequence identity to SEQ ID NO: 4; and
a deletion in a gene encoding an S8-SFB amino acid sequence with at least 90%, at least 95%, at least 99% or 100% sequence identity to SEQ ID NO: 5.

89. The self-fertile almond plant of claim 87, wherein said deletion comprises:
a deletion of an S7-RNAse gene with a transcribed portion, wherein the transcribed portion comprises a DNA sequence with at least 90%, at least 95%, at least 99% or 100% sequence identity to SEQ ID NO: 6;
a deletion of an S7-RNAse gene comprising a DNA protein coding sequence with at least 90%, at least 95%, at least 99% or 100% sequence identity to SEQ ID NO: 7;
a deletion of a gene encoding an S7-RNAse amino acid sequence with at least 90%, at least 95%, at least 99% or 100% sequence identity to SEQ ID NO: 8;
a deletion in an S7-SFB gene with a transcribed portion, wherein the transcribed portion comprises a DNA sequence with at least 90%, at least 95%, at least 99% or 100% sequence identity to SEQ ID NO: 9; and
a deletion in a gene encoding an S7-SFB amino acid sequence that has at least 90%, at least 95%, at least 99% or 100% sequence identity to SEQ ID NO: 10.

90. The self-fertile almond plant of claim 87, wherein the one or more S-locus haplotypes comprise a deletion of two or more genes.

91. The self-fertile almond plant of claim 90, wherein said deletion of two or more genes comprise:
an S8-locus comprising a gene encoding a deletion of an S8-RNase gene with a transcribed portion, wherein the transcribed portion comprises a DNA sequence with at least 90%, at least 95%, at least 99% or 100% sequence identity to SEQ ID NO: 1; a deletion of an S8-RNase gene comprising a DNA protein coding sequence with at least 90%, at least 95%, at least 99% or 100% sequence identity to SEQ ID NO: 2; and a deletion of a gene encoding an S8-RNase amino acid sequence with at least 90%, at least 95%, at least 99% or 100% sequence identity to SEQ ID NO: 3; and
an S7-locus comprising a gene encoding a deletion of an S7-RNAse gene with a transcribed portion, wherein the transcribed portion comprises a DNA sequence with at least 90%, at least 95%, at least 99% or 100% sequence identity to SEQ ID NO: 6; a deletion of an S7-RNAse gene comprising a DNA protein coding sequence with at least 90%, at least 95%, at least 99% or 100% sequence identity to SEQ ID NO: 7; and a deletion of a gene encoding an S7-RNAse amino acid sequence with at least 90%, at least 95%, at least 99% or 100% sequence identity to SEQ ID NO: 8.

92. The self-fertile plant of claim 88, wherein the deletion begins in a region between about 20 bp upstream and about 20 bp downstream from the 3′ end of a target site for a guided nuclease.

93. The self-fertile almond plant of claim 92, wherein the plant contains a gRNA target site for a guided nuclease, wherein the guided nuclease comprises a guide nucleic acid, and wherein the guide nucleic acid comprises a guide RNA.

94. The self-fertile almond plant of claim 93, wherein the guide RNA comprises an RNA sequence with at least 90%, at least 95%, at least 99% or 100% sequence identity to a sequence selected from the group consisting of SEQ ID NO: 11, SEQ ID NO: 12, SEQ ID NO: 13, SEQ ID NO: 14, SEQ ID NO: 15 SEQ ID NO: 16, SEQ ID NO: 17, SEQ ID NO: 18, SEQ ID NO: 19, SEQ ID NO: 20, SEQ ID NO: 21 SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96 and SEQ ID NO: 97 and SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133 and SEQ ID NO: 134.

95. The self-fertile almond plant of claim 94, wherein the target site comprises a DNA sequence with at least 90%, at least 95%, at least 99% or 100% sequence identity to a sequence selected from the group consisting of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118 and SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, and SEQ ID NO: 150.

96. A processed almond product derived from the self-fertile almond plant of claim 95, wherein the processed almond product comprises a detectable amount of at least one of the one or more S-locus haplotypes of the self-fertile almond plant.

97. The self-fertile plant of claim 91, wherein the deletion begins in a region between about 20 bp upstream and about 20 bp downstream from the 3′ end of a target site for a guided nuclease.

98. The self-fertile almond plant of claim 97, wherein the plant contains a gRNA target site for a guided nuclease, wherein the guided nuclease comprises a guide nucleic acid, and wherein the guide nucleic acid comprises a guide RNA.

99. The self-fertile almond plant of claim 98, wherein the guide RNA comprises an RNA sequence with at least 90%, at least 95%, at least 99% or 100% sequence identity to a sequence selected from the group consisting of SEQ ID NO: 11, SEQ ID NO: 12, SEQ ID NO: 13, SEQ ID NO: 14, SEQ ID NO: 15 SEQ ID NO: 16, SEQ ID NO: 17, SEQ ID NO: 18, SEQ ID NO: 19, SEQ ID NO: 20, SEQ ID NO: 21 SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96 and SEQ ID NO: 97 and SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133 and SEQ ID NO: 134.

100. The self-fertile almond plant of claim 99, wherein the target site comprises a DNA sequence with at least 90%, at least 95%, at least 99% or 100% sequence identity to a sequence selected from the group consisting of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118 and SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, and SEQ ID: 150.

101. A processed almond product derived from the self-fertile almond plant of claim 100, wherein the processed almond product comprises a detectable amount of at least one of the one or more S-locus haplotypes of the self-fertile almond plant.

102. A method of producing a self-fertile almond plant, plant part, or plant cell, the method comprising:
introducing into an almond cell one or more S-locus haplotypes, wherein the one or more S-locus haplotypes comprise a deletion that results in a frameshift mutation or an in-frame mutation in one or more S-locus genes; and
regenerating a modified almond plant, plant, part, or plant cell from the almond cell, wherein the modified almond plant, plant, part, or plant cell comprises one or more S-locus haplotypes, and wherein one or more genes within the one or more S-locus haplotypes comprise a deletion and wherein said deletion results in a frameshift mutation and/or an in-frame mutation.

103. The method of claim 102, wherein said deletion comprises:
a deletion of an S8-RNase gene with a transcribed portion, wherein the transcribed portion comprises a DNA sequence with at least 90%, at least 95%, at least 99% or 100% sequence identity to SEQ ID NO: 1;
a deletion of an S8-RNase gene comprising a DNA protein coding sequence with at least 90%, at least 95%, at least 99% or 100% sequence identity to SEQ ID NO: 2;
a deletion of a gene encoding an S8-RNase amino acid sequence with at least 90%, at least 95%, at least 99% or 100% sequence identity to SEQ ID NO: 3;
a deletion of an S8-SFB gene with a transcribed portion, wherein the transcribed portion comprises a DNA sequence with at least 90%, at least 95%, at least 99% or 100% sequence identity to SEQ ID NO: 4; and
a deletion in a gene encoding an S8-SFB amino acid sequence with at least 90%, at least 95%, at least 99% or 100% sequence identity to SEQ ID NO: 5.

104. The method of claim 102, wherein said deletion comprises:
a deletion of an S7-RNAse gene with a transcribed portion, wherein the transcribed portion comprises a DNA sequence with at least 90%, at least 95%, at least 99% or 100% sequence identity to SEQ ID NO: 6;
a deletion of an S7-RNAse gene comprising a DNA protein coding sequence with at least 90%, at least 95%, at least 99% or 100% sequence identity to SEQ ID NO: 7;
a deletion of a gene encoding an S7-RNAse amino acid sequence with at least 90%, at least 95%, at least 99% or 100% sequence identity to SEQ ID NO: 8;
a deletion in an S7-SFB gene with a transcribed portion, wherein the transcribed portion comprises a DNA sequence with at least 90%, at least 95%, at least 99% or 100% sequence identity to SEQ ID NO: 9; and
a deletion in a gene encoding an S7-SFB amino acid sequence that has at least 90%, at least 95%, at least 99% or 100% sequence identity to SEQ ID NO: 10.

105. The method of claim 102, wherein the one or more S-locus haplotypes comprise a deletion of two or more genes.

106. The method of claim 105, wherein said deletion of two or more genes comprise:
an S8-locus comprising a gene encoding a deletion of an S8-RNase gene with a transcribed portion, wherein the transcribed portion comprises a DNA sequence with at least 90%, at least 95%, at least 99% or 100% sequence identity to SEQ ID NO: 1; a deletion of an S8-RNase gene comprising a DNA protein coding sequence with at least 90%, at least 95%, at least 99% or 100% sequence identity to SEQ ID NO: 2; and a deletion of a gene encoding an S8-RNase amino acid sequence with at least 90%, at least 95%, at least 99% or 100% sequence identity to SEQ ID NO: 3; and
an S7-locus comprising a gene encoding a deletion of an S7-RNAse gene with a transcribed portion, wherein the transcribed portion comprises a DNA sequence with at least 90%, at least 95%, at least 99% or 100% sequence identity to SEQ ID NO: 6; a deletion of an S7-RNAse gene comprising a DNA protein coding sequence with at least 90%, at least 95%, at least 99% or 100% sequence identity to SEQ ID NO: 7; and a deletion of a gene encoding an S7-RNAse amino acid sequence with at least 90%, at least 95%, at least 99% or 100% sequence identity to SEQ ID NO: 8.

107. The method of claim 106, wherein the deletion begins in a region between about 20 bp upstream and about 20 bp downstream from the 3′ end of a target site for a guided nuclease.

108. The method of claim 107, wherein the plant contains a gRNA target site for a guided nuclease, wherein the guided nuclease comprises a guide nucleic acid, and wherein the guide nucleic acid comprises a guide RNA.

109. The method of claim 108, wherein the guide RNA comprises an RNA sequence with at least 90%, at least 95%, at least 99% or 100% sequence identity to a sequence selected from the group consisting of SEQ ID NO: 11, SEQ ID NO: 12, SEQ ID NO: 13, SEQ ID NO: 14, SEQ ID NO: 15 SEQ ID NO: 16, SEQ ID NO: 17, SEQ ID NO: 18, SEQ ID NO: 19, SEQ ID NO: 20, SEQ ID NO: 21 SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96 and SEQ ID NO: 97 and SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133 and SEQ ID NO: 134.

110. The method of claim 109, wherein the target site comprises a DNA sequence with at least 90%, at least 95%, at least 99% or 100% sequence identity to a sequence selected from the group consisting of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118 and SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, and SEQ ID NO: 150.

111. A processed almond product derived from a self-fertile almond produced from the method of claim 110, wherein the processed almond product comprises a detectable amount of at least one of the one or more S-locus haplotypes of the self-fertile almond plant.

112. The method of claim 103, wherein the deletion begins in a region between about 20 bp upstream and about 20 bp downstream from the 3′ end of a target site for a guided nuclease.

113. The method of claim 112, wherein the plant contains a gRNA target site for a guided nuclease, wherein the guided nuclease comprises a guide nucleic acid, wherein the guide nucleic acid comprises a guide RNA.

114. The method of claim 113, wherein the guide RNA comprises an RNA sequence with at least 90%, at least 95%, at least 99% or 100% sequence identity to a sequence selected from the group consisting of SEQ ID NO: 11, SEQ ID NO: 12, SEQ ID NO: 13, SEQ ID NO: 14, SEQ ID NO: 15 SEQ ID NO: 16, SEQ ID NO: 17, SEQ ID NO: 18, SEQ ID NO: 19, SEQ ID NO: 20, SEQ ID NO: 21 SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96 and SEQ ID NO: 97 and SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133 and SEQ ID NO: 134.

115. The method of claim 114, wherein the target site comprises a DNA sequence with at least 90%, at least 95%, at least 99% or 100% sequence identity to a sequence selected from the group consisting of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118 and SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, and SEQ ID NO: 150.

116. A recombinant DNA construct comprising a first expression cassette comprising a first DNA sequence encoding a guide RNA, wherein said guide RNA comprises an RNA sequence with at least 90%, at least 95%, at least 99% or 100% sequence identity to a sequence selected from the group consisting of SEQ ID NO: 11, SEQ ID NO: 12, SEQ ID NO: 13, SEQ ID NO: 14, SEQ ID NO: 15 SEQ ID NO: 16, SEQ ID NO: 17, SEQ ID NO: 18, SEQ ID NO: 19, SEQ ID NO: 20, SEQ ID NO: 21 SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96 and SEQ ID NO: 97 and SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133 and SEQ ID NO: 134.
